(19) United States
(12) Patent Application Publication
(10) Pub. No.: US 2015/0361494 A1
(43) Pub. Date: Dec. 17, 2015

(54) Genetic Markers Associated with Endometriosis and Use Thereof

(71) Applicants: Kenneth Ward, Salt Lake City, UT (US); Hans Albertson, Salt Lake City, UT (US); Georges C. Frech, Salt Lake City, UT (US); Rakesh N. Chettier, West Jordan, UT (US)

(72) Inventors: Kenneth Ward, Salt Lake City, UT (US); Hans Albertson, Salt Lake City, UT (US); Georges C. Frech, Salt Lake City, UT (US); Rakesh N. Chettier, West Jordan, UT (US)

(73) Assignee: JUNEAU BIOSCIENCES, LLC, Salt Lake city, UT (US)

(21) Appl. No.: 13/159,132

(22) Filed: Jun. 13, 2011

Related U.S. Application Data
(63) Continuation of application No. 13/071,598, filed on Mar. 25, 2011, now abandoned.
(60) Provisional application No. 61/318,145, filed on Mar. 26, 2010.

(51) Int. Cl. C40B 30/04; A61K 38/02; G06F 19/24; A61K 39/395; A61K 31/7088; A61P 15/00; A61K 48/00

(52) U.S. Cl. CPC C12Q 1/6883; C12Q 2600/172; C12Q 2600/156; C12Q 2600/106

(57) ABSTRACT

The present invention relates to novel genetic markers associated with endometriosis and risk of developing endometriosis, and methods and materials for determining whether a human subject has endometriosis or is at risk of developing endometriosis and the use of such risk information in selectively administering a treatment that at least partially prevents or compensates for an endometriosis related symptom.

### CROSS REFERENCE TO RELATED APPLICATIONS

This nonprovisional utility patent application is a continuation of and claims the benefit under 35 U.S.C. §120 to co-pending U.S. application Ser. No. 13/071,598 filed Mar. 25, 2011, which claims the benefit under 35 U.S.C. §119(e) of U.S. provisional application No. 61/318,145, filed Mar. 26, 2010, all of which are incorporated, in their entirety, by this reference.

### FIELD OF THE INVENTION

The present invention relates to endometriosis diagnosis and therapy. In particular, the present invention relates to specific single nucleotide polymorphisms (SNPs) in the human genome, and their association with endometriosis and related pathologies.

### BACKGROUND OF THE INVENTION

Endometriosis in one instance refers to autoimmune endometriosis, mild endometriosis, moderate endometriosis or severe endometriosis. For the purpose of this invention the term endometriosis is used to describe any of these conditions.

Endometriosis is most generally defined as the presence of endometrium (glands and stroma) at sites outside of the uterus (ectopic endometrial tissues rather than eutopic or within the uterus). The most common sites are the ovaries, pelvic peritoneum, uterosacral ligaments, pouch of Douglas, and rectovaginal septum although implants have been identified on the peritoneal surfaces of the abdomen (these may grow into the intestines, ureters or bladder), in the thorax, at the umbilicus, and at incision sites of prior surgeries (Child T J, Tan S L (2001) Endometriosis: aetiology, pathogenesis and treatment. Drugs 61:1735-1750; Giudice et al. (1998) Status of current research on endometriosis. The Journal of reproductive medicine 43:252-262).

Endometriosis is a common gynecologic disorder. The prevalence is difficult to know. It has been estimated that it affects approximately 14% of all women (range 1-43%), 40-60% of women with pelvic pain and 30%-50% of infertile women (Di Blasio et al. (2005) Genetics of endometriosis. Minerva ginecologica 57:225-236; Schindler A E (2004) Pathophysiology, diagnosis and treatment of endometriosis. Minerva ginecologica 56:419-435).

Studies of the inheritance of endometriosis have been hampered by methodological problems related to disease definition and control selection. General population incidence during the 1970s in this country has been suggested to be 1.6 per 1000 white females aged 15-49, while a more current study based upon hospital discharges finds endometriosis as a first listed diagnosis in 1.3 per 1000 discharges in women aged 15-44. There is a clinical impression that blacks have lower rates of endometriosis and Orientals have higher rates than whites. Separate work has suggested a polygenic/multifactorial inheritance (Vigano P, Somigliana E, Vignali M, Busacca M, Blasio A M (2007) Genetics of endometriosis: current status and prospects. Front Biosci 12:3247-3255). Affected sib-pair studies have also been performed (Kennedy et al. (2001) Affected sib-pair analysis in endometriosis. Human reproduction update 7:411-418; Treloar et al. (2005) Genomewide linkage study in 1,176 affected sister pair families identifies a significant susceptibility locus for endometriosis on chromosome 10q26. Am J Hum Genet 77:365-376).

Specific genes with polymorphisms have been investigated for an association with endometriosis. Some association studies implicated GALT (a gene involved in galactose metabolism), and GSTM1 and NAT2 (genes encoding for the detoxification enzymes) as possible disease susceptibility genes. Recent findings have added to the evidence for the involvement of GSTM1 and NAT2, but have cast doubt on the role of GALT. The p21 gene codon 31 arginine/serine polymorphism is not associated with endometriosis.

Polymorphisms of the arylhydrocarbon receptor (AHR) gene and related genes were examined, and in at least one study, no association was found. However, the design of many genetic and epidemiological studies has been inadequate with respect to sample size, consistency in phenotype definition, and the choice of control populations. To identify genomic changes involved in the development of endometriosis (Gogusev et al. (1999). “Detection of DNA copy number changes in human endometriosis by comparative genomic hybridization.” Hum Genet 105(5): 444-51) examined endometriotic tissues by comparative genomic hybridization and detected losses of 1p and 22q in 50% of the cases. Additional common losses included 7p (22%). Dual-color FISH using probes for the deleted regions on chromosomes 1, 7, and 22 supported the CGH data. Treloar et al. (Treloar et al. (2005). “Genomewide linkage study in 1,176 affected sister pair families identifies a significant susceptibility locus for endometriosis on chromosome 10q26.” Am J Hum Genet 77(3): 365-76) conducted a linkage study of 1,176 families (931 Australian and 245 from the U.K.), each with at least 2 affected family members, usually affected sister pairs, with surgically diagnosed disease. They identified a region of significant linkage on 10q26 (maximum lod score=3.09; genomewide P=0.047) and another region of suggestive linkage on 20p13; minor peaks were found on 8 other chromosomes.

Endometriosis is a genetically inherited disease. Genetic variation in DNA sequences is often associated with heritable phenotypes, such as an individual's propensity towards complex disorders. Single nucleotide polymorphisms are the most common form of genetic sequence variations. Detection and analysis of specific genetic mutations, such as single nucleotide polymorphisms (SNPs), which are associated with endometriosis risk, may therefore be used to determine risk of endometriosis, the presence of endometriosis or the progression of endometriosis. Genetic markers that are prognostic for endometriosis can be genotyped early in life and could predict individual response to various risk factors and treatment. Genetic predisposition revealed by genetic analysis of susceptibility genes can provide an integrated assessment of the interaction between genotypes and environmental factors, resulting in synergistically increased prognostic value of diagnostic tests. Thus, pre-symptomatic and early symptomatic genetic testing is expected to be the cornerstone of the paradigmatic shift from late surgical interventions to earlier preventative therapies.

Thus, there is an urgent need for novel genetic markers that are predictive of endometriosis and endometriosis progression, particularly in treatment decisions for individuals who are recognized as having endometriosis. Such genetic markers may enable prognosis of endometriosis in much larger populations compared with the populations which can currently be evaluated by using existing risk factors and biomarkers. The availability of a genetic test may allow, for example, early diagnosis and prognosis of endometriosis, as well as early clinical intervention to mitigate progression of the disease. The use of these genetic markers will also allow selection of subjects for clinical trials involving novel treatment methods. The discovery of genetic markers associated with endometriosis will further provide novel targets for therapeutic intervention or preventive treatments of endometriosis and enable the development of new therapeutic agents for treating endometriosis.

### SUMMARY OF THE INVENTION

The present invention relates to the identification of novel SNPs, unique combinations of such SNPs, and haplotypes of SNPs that are associated with endometriosis and related pathologies. The polymorphisms disclosed herein are directly useful as targets for the design of diagnostic reagents and the development of therapeutic agents for use in the diagnosis and treatment of endometriosis and related pathologies.

Based on the identification of SNPs associated with endometriosis, the present invention also provides methods of detecting these variants as well as the design and preparation of detection reagents needed to accomplish this task. The invention specifically provides novel SNPs in genetic sequences involved in endometriosis, methods of detecting these SNPs in a test sample, methods of identifying individuals who have an altered risk of developing endometriosis and for suggesting treatment options for endometriosis based on the presence of a SNP(s) disclosed herein or its encoded product, and methods of identifying individuals who are more or less likely to respond to a treatment.

In one embodiment of the invention, the present invention provides SNPs, as set forth in Table 1 having significant association with endometriosis by allelic, Cochran-Armitage trend, genotypic, dominant and/or recessive gene action test, or by being co-located within the same LD blocks as the SNPs listed in Tables 1, and such as set forth in Tables 3-433. Tables 3-433 provide information identifying SNPs of the present invention, including SNP “rs” identification numbers (a reference SNP or RefSNP accession ID number) or “SNP-A” identification numbers (a reference SNP or RefSNP accession ID number), chromosome number, and base position number of the SNP.

In a specific embodiment of the present invention, naturally-occurring SNPs in the human genome are provided that are associated with endometriosis. Such SNPs can have a variety of uses in the diagnosis and/or treatment of endometriosis. One aspect of the present invention relates to an isolated nucleic acid molecule comprising a nucleotide sequence in which at least one nucleotide is a SNP disclosed in Table 1. In an alternative embodiment, a nucleic acid of the invention is an amplified polynucleotide, which is produced by amplification of a SNP-containing nucleic acid template.

In yet another embodiment of the invention, a reagent for detecting a SNP in the context of its naturally-occurring flanking nucleotide sequences (which can be, e.g., either DNA or mRNA) is provided. In particular, such a reagent may be in the form of, for example, a hybridization probe or an amplification primer that is useful in the specific detection of a SNP of interest.

Also provided in the invention are kits comprising SNP detection reagents and methods for detecting the SNPs disclosed herein by employing detection reagents. In a specific embodiment, the present invention provides for a method of identifying an individual having an increased or decreased risk of developing endometriosis by detecting the presence or absence of a SNP allele disclosed herein. In another embodiment, a method for diagnosis of endometriosis by detecting the presence or absence of a SNP allele disclosed herein is provided. In yet another embodiment a method for predicting endometriosis sub-classification by detecting the presence or absence of a SNP allele disclosed herein is provided.

In yet another embodiment, the invention also provides a kit comprising SNP detection reagents, and methods for detecting the SNPs disclosed herein by employing detection reagents and a questionnaire of non-genetic clinical factors. In one embodiment, the questionnaire would be completed by a medical professional based on medical history physical exam or other clinical findings. In yet another embodiment, the questionnaire would include any other non-genetic clinical factors known to be associated with the risk of developing endometriosis.

Many other uses and advantages of the present invention will be apparent to those skilled in the art upon review of the detailed description of the preferred embodiments herein. Solely for clarity of discussion, the invention is described in the sections below by way of non-limiting examples.

### DETAILED DESCRIPTION OF THE INVENTION

### Definitions

The term “haplotype” means a combination of genotypes on the same chromosome occurring in a linkage disequilibrium block. Haplotypes serve as markers for linkage disequilibrium blocks, and at the same time provide information about the arrangement of genotypes within the blocks. Typing of only certain SNPs which serve as tags can, therefore, with a high level of precision reveal all genotypes for SNPs located within a block. Thus, the use of haplotypes greatly facilitates identification of candidate genes associated with diseases and drug sensitivity.

The term “linkage disequilibrium” or “LD” means that a particular combination of alleles (alternative nucleotides) or genetic markers at two or more different SNP sites within a given chromosomal region are non-randomly co-inherited, meaning that the combination of alleles at the different SNP sites occurs more or less frequently in a population than the separate frequencies of occurrence of each allele or the frequency of a random formation of haplotypes from alleles in a given population. “LD” differs from “linkage,” which describes the association of two or more loci on a chromosome with limited recombination between them. LD is also used to refer to any non-random genetic association between allele(s) at two or more different SNP sites. Therefore, when a SNP is in LD with other SNPs, the particular allele of the first SNP often predicts which alleles will be present in those SNPs in LD. LD is generally due to the physical proximity of the two loci along a chromosome. Hence, genotyping one of the SNP sites will give almost the same information as genotyping the other SNP site that is in LD. LD is caused by fitness interactions between genes or by such non-adaptive processes as population structure, inbreeding, and stochastic effects.

Various degrees of LD can be encountered between two or more SNPs with the result being that some SNPs are more closely associated (i.e., in stronger LD) than others. Furthermore, the physical distance over which LD extends along a chromosome differs between different regions of the genome, and therefore the degree of physical separation between two or more SNP sites necessary for LD to occur can differ between different regions of the genome. The average LD block size in Caucasians has been estimated to 16.3 kb occasionally extending across several hundred kb. LD blocks may also vary in size between ethnic groups (The International HapMap Consortium. A haplotype map of the human genome. Nature. Oct. 27, 2005; 437(7063):1299-1320). Conservatively, LD can be defined as SNPs that have a D prime value of 1 and a LOD score greater than 2.0 or an r-squared value greater than 0.8.

“Linkage disequilibrium block” or “LD block” means a region of the genome that contains multiple SNPs located in proximity to each other and that are transmitted as a block. Each of tables 003 through 433 disclose markers residing in the indicated LD block and provide end points delineating the span of the region of the indicated LD block. An LD block span may be defined by a first and second end point of the LD block. Thus for instance, LD block 001 of table 003 may be defined as an LD block having a first end point of base position 3,290,990 and a second end point of base position 3,309,924 resulting in a region span of 18,934 (i.e. 3,309,924 minus U.S. Pat. No. 3,290,990).

“D prime” or “D′” (also referred to as the “linkage disequilibrium measure” or “linkage disequilibrium parameter”) means the deviation of the observed allele frequencies from the expected, and is a statistical measure of how well a biometric system can discriminate between different individuals. The larger the D′ value, the better a biometric system is at discriminating between individuals.

“LOD score” is the “logarithm of the odd” score, which is a statistical estimate of whether two genetic loci are physically near enough to each other (or “linked”) on a particular chromosome that they are likely to be inherited together. A LOD score of three or more is generally considered statistically significant evidence of linkage.

“R-squared” or “r2” (also referred to as the “correlation coefficient”) is a statistical measure of the degree to which two markers are related. The nearer to 1.0 the r2 value is, the more closely the markers are related to each other. R2 cannot exceed 1.0. D prime and LOD scores generally follow the above definition for SNPs in LD. R2, however, displays a more complex pattern and can vary between about 0.0003 and 1.0 in SNPs that are in LD.

The present invention provides SNPs associated with endometriosis, nucleic acid molecules containing SNPs, methods and reagents for the detection of the SNPs disclosed herein, uses of these SNPs for the development of detection reagents, and assays or kits that utilize such reagents. The SNPs disclosed herein are useful for diagnosing, screening for, and evaluating predisposition to endometriosis and progression of endometriosis. Additionally, such SNPs are useful in the determining individual subject treatment plans and design of clinical trials of devices for possible use in the treatment of endometriosis. Furthermore, such SNPs and their encoded products are useful targets for the development of therapeutic agents. Furthermore, such SNPs combined with other non-genetic clinical factors are useful for diagnosing, screening, evaluating predisposition to endometriosis, assessing risk of progression of endometriosis, determining individual subject treatment plans and design of clinical trials of devices for possible use in the treatment of endometriosis.

SNPs

As used herein, the term “SNP” refers to single nucleotide polymorphisms in DNA. SNPs are usually preceded and followed by highly conserved sequences that vary in less than 1/100 or 1/1000 members of the population. An individual may be homozygous or heterozygous for an allele at each SNP position. A SNP may, in some instances, be referred to as a “cSNP” to denote that the nucleotide sequence containing the SNP is an amino acid “coding” sequence.

A SNP may arise from a substitution of one nucleotide for another at the polymorphic site. Substitutions can be transitions or transversions. A transition is the replacement of one purine nucleotide by another purine nucleotide, or one pyrimidine by another pyrimidine. A transversion is the replacement of a purine by a pyrimidine, or vice versa. A SNP may also be a single base insertion or deletion variant referred to as an “indel”.

A synonymous codon change, or silent mutation SNP (terms such as “SNP,” “polymorphism,” “mutation,” “mutant,” “variation,” and “variant” are used herein interchangeably), is one that does not result in a change of amino acid due to the degeneracy of the genetic code. A substitution that changes a codon coding for one amino acid to a codon coding for a different amino acid (i.e., a non-synonymous codon change) is referred to as a missense mutation. A nonsense mutation results in a type of non-synonymous codon change in which a stop codon is formed, thereby leading to premature termination of a polypeptide chain and a truncated protein. A read-through mutation is another type of non-synonymous codon change that causes the destruction of a stop codon, thereby resulting in an extended polypeptide product. An indel that occurs in a coding DNA segment gives rise to a frameshift mutation. While SNPs can be bi-, tri-, or tetra-allelic, the vast majority of the SNPs are bi-allelic, and are thus often referred to as “bi-allelic markers,” or “di-allelic markers”.

As used herein, references to SNPs and SNP genotypes include individual SNPs and/or haplotypes, which are groups of SNPs that are generally inherited together. Haplotypes can have stronger correlations with diseases or other phenotypic effects compared with individual SNPs, and therefore may provide increased diagnostic accuracy in some cases.

Causative SNPs are those SNPs that produce alterations in gene expression or in the structure and/or function of a gene product, and therefore are predictive of a possible clinical phenotype. One such class includes SNPs falling within regions of genes encoding a polypeptide product, i.e. cSNPs. These SNPs may result in an alteration of the amino acid sequence of the polypeptide product (i.e., non-synonymous codon changes) and give rise to the expression of a defective or other variant protein. Furthermore, in the case of nonsense mutations, a SNP may lead to premature termination of a polypeptide product. Such variant products can result in a pathological condition, e.g., genetic endometriosis.

Causative SNPs do not necessarily have to occur in coding regions; causative SNPs can occur in, for example, any genetic region that can ultimately affect the expression, structure, and/or activity of the protein encoded by a nucleic acid. Such genetic regions include, for example, those involved in transcription, such as SNPs in transcription factor binding domains, SNPs in promoter regions, in areas involved in transcript processing, such as SNPs at intron-exon boundaries that may cause defective splicing, or SNPs in mRNA processing signal sequences such as polyadenylation signal regions and miRNA recognition sites. Some SNPs that are not causative SNPs nevertheless are in close association with, and therefore segregate with, a disease-causing sequence. In this situation, the presence of a SNP correlates with the presence of, or predisposition to, or an increased risk in developing the endometriosis. These SNPs, although not causative, are nonetheless also useful for diagnostics, endometriosis predisposition screening, endometriosis progression risk and other uses.

Methods

An association study of a SNP and a specific disorder involves determining the presence or frequency of the SNP allele in biological samples from individuals with the disorder of interest, such as endometriosis, and comparing the information to that of controls (i.e., individuals who do not have the disorder; controls may be also referred to as “healthy” or “normal” individuals) who are preferably of similar age and race. The appropriate selection of patients and controls is important to the success of SNP association studies. Therefore, a pool of individuals with well-characterized phenotypes is extremely desirable.

A SNP may be screened in tissue samples or any biological sample obtained from an affected individual, and compared to control samples, and selected for its increased (or decreased) occurrence in a specific pathological condition, such as pathologies related to endometriosis. Once a statistically significant association is established between one or more SNP(s) and a pathological condition (or other phenotype) of interest, then the region around the SNP can optionally be thoroughly screened to identify the causative genetic locus/sequence(s) (e.g., causative SNP/mutation, gene, regulatory region, etc.) that influences the pathological condition or phenotype. Association studies may be conducted within the general population and are not limited to studies performed on related individuals in affected families (linkage studies). For diagnostic and prognostic purposes, if a particular SNP site is found to be useful for diagnosing a disease, such as endometriosis, other SNP sites which are in LD with this SNP site would also be expected to be useful for diagnosing the condition. Linkage disequilibrium is described in the human genome as blocks of SNPs along a chromosome segment that do not segregate independently (i.e., that are non-randomly co-inherited). The starting (5′ end) and ending (3′ end) of these blocks can vary depending on the criteria used for linkage disequilibrium in a given database, such as the value of D′ or r2 used to determine linkage disequilibrium.

Table 1 discloses SNPs that have been shown in case-control studies to be associated with endometriosis. Table 1 specifically shows SNPs from the Affymetrix 6.0 GeneChip that all showed significant association with endometriosis and identifying information regarding each SNP in columns labeled “dbSNPrsID” (the NCBI reference SNP identifier), “Chr” (the Chromosome where the SNP is located; note that the chromosome numbered “23” is used interchangeably for chromosome “X”), “Position” (the basepair position on the chromosome indicated, based on the NCBI Genome Build 36), “OR” (the Odds Ratio for the SNP in question), “F_A” (the minor allele frequency observed in the endometriosis affected cases), “F_U” (the minor allele frequency observed in the control individuals), “P-Value” (the lowest p-value based on either Allelic, Genotypic, Cochran-Armitage Trend, Genotypic, Dominant and/or Recessive gene action test as calculated by PLINK), “MA” (the minor allele nucleotide at the SNP position), and “Flanking Sequence” (the DNA sequence surrounding the SNP in question; the two allelic variants observed for the SNP are indicated in square brackets in the middle of the sequence). Table 1A lists 233 SNPs significant for association with endometriosis from an analysis that involved 1490 endometriosis cases and 1469 matched population controls. Table 1B lists 267 SNPs significant for association with endometriosis from an analysis that involved 622 endometriosis cases and 1469 matched population controls; for this analysis the cases' diagnoses were confirmed by histological examination of endometriotic lesions AND/OR their disease severity was either moderate or severe. Table 1C lists 233 SNPs significant for association with endometriosis from an analysis with 1088 endometriosis cases and 934 controls, an analysis that used more stringent filtering based on ethnicity as well as Affymetrix 6.0 microarray experiment quality. The following filters were used for all analyses: SNP markers missing per sample: <10%, samples missing per SNP marker: <4%, Hardy-Weinberg Equilibrium p-value in the control population: >0.001, Minor Allele (MA) frequency: >3%, and cases versus controls differential SNP marker failure p-value: >0.001

Observed heterozygosity: <60% Tables 3-433 define the linkage disequilibrium blocks surrounding each of the 656 non-redundant SNPs identified in Table 1. The linkage disequilibrium blocks were ascertained based upon the criteria set forth by the Haploview (ver. 4.2) computer algorithm under default settings (Confidence Intervals algorithm implemented by Gabriel et al. “The structure of haplotype blocks in the human genome.” Science 296:2225-9, 2002) using the HapMap dataset release 27 (Barrett J C. “Haploview: Visualization and analysis of SNP genotype data.” CSH Protoc. 2009 October; 2009(10):pdb.ip71). It is noted however, that there may be other SNPs located in the regions covered by the LD blocks but not specifically listed in the LD block tables herein that due to their being located in the same region as SNPs in the LD blocks, have an association with endometriosis and are likewise valuable in determining predisposition to endometriosis.

Each of Tables 3-433 is prefaced by one or more SNPs from Table 1, and includes a list of one or more SNPs that correspond to a linkage disequilibrium block, including the SNPs from Table 1, which are highlighted in bold character within the table. Occasionally, an original SNP marker may not itself be present in the Hapmap SNP list but its presence is inferred based on its chromosomal location and basepair position. Also indicated in the tables is the chromosome and physical position in basepairs. On rare occasions, a SNP falls outside of a linkage disequilibrium block, in which case no LD block is presented.

The SNPs shown in Tables 1-433 may be useful individually, in combination with one of the other SNPs or in a haplotype involving one of the other SNPs in Tables 1-433 and/or other DNA markers present within the LD block. Linkage disequilibrium blocks can be determined from genomewide genetic population studies which results are accessible in private and public databases, and can be visualized or tabularized using, for example, the Haploview software (Barrett J C. Haploview: Visualization and analysis of SNP genotype data. CSH Protoc. 2009 October; 2009(10):pdb.ip71). The linkage disequilibrium blocks described in Tables 1-433 were identified using Haploview version 4.2 based on the International HapMap Consortium data release 27.

In accordance with the present invention, SNPs have been identified in a study using a whole-genome case-control approach to identify single nucleotide polymorphisms that were closely associated with the development of endometriosis, as well as SNPs found to be in linkage disequilibrium with (i.e., within the same linkage disequilibrium block as) the endometriosis-associated SNPs, which can provide haplotypes (i.e., groups of SNPs that are co-inherited) to be readily inferred. Thus, the present invention provides individual SNPs associated with endometriosis, as well as combinations of SNPs and haplotypes in genetic regions associated with endometriosis, methods of detecting these polymorphisms in a test sample, methods of determining the risk of an individual of having or developing endometriosis and for clinical sub-classification of endometriosis.

The present invention also provides SNPs associated with endometriosis, as well as SNPs that were previously known in the art, but were not previously known to be associated with endometriosis. Accordingly, the present invention provides novel compositions and methods based on the SNPs disclosed herein, and also provides novel methods of using the known but previously unassociated SNPs in methods relating to endometriosis (e.g., for diagnosing endometriosis. etc.).

Particular SNP alleles of the present invention can be associated with either an increased risk of having or developing endometriosis, or a decreased risk of having or developing endometriosis. SNP alleles that are associated with a decreased risk may be referred to as “protective” alleles, and SNP alleles that are associated with an increased risk may be referred to as “susceptibility” alleles, “risk factors”, or “high-risk” alleles. Thus, whereas certain SNPs can be assayed to determine whether an individual possesses a SNP allele that is indicative of an increased risk of having or developing endometriosis (i.e., a susceptibility allele), other SNPs can be assayed to determine whether an individual possesses a SNP allele that is indicative of a decreased risk of having or developing endometriosis (i.e., a protective allele). Similarly, particular SNP alleles of the present invention can be associated with either an increased or decreased likelihood of responding to a particular treatment. The term “altered” may be used herein to encompass either of these two possibilities (e.g., an increased or a decreased risk/likelihood).

Those skilled in the art will readily recognize that nucleic acid molecules may be double-stranded molecules and that reference to a particular site on one strand refers, as well, to the corresponding site on a complementary strand. In defining a SNP position, SNP allele, or nucleotide sequence, reference to an adenine, a thymine (uridine), a cytosine, or a guanine at a particular site on one strand of a nucleic acid molecule also defines the complementary thymine (uridine), adenine, guanine, or cytosine (respectively) at the corresponding site on a complementary strand of the nucleic acid molecule. Thus, reference may be made to either strand in order to refer to a particular SNP position, SNP allele, or nucleotide sequence. Probes and primers may be designed to hybridize to either strand and SNP genotyping methods disclosed herein may generally target either strand. Throughout the specification, in identifying a SNP position, reference is generally made to the forward or “sense” strand, solely for the purpose of convenience. Since endogenous nucleic acid sequences exist in the form of a double helix (a duplex comprising two complementary nucleic acid strands), it is understood that the SNPs disclosed herein will have counterpart nucleic acid sequences and SNPs associated with the complementary “reverse” or “antisense” nucleic acid strand. Such complementary nucleic acid sequences, and the complementary SNPs present in those sequences, are also included within the scope of the present invention.

Isolated Nucleic Acid Molecules

The present invention provides isolated nucleic acid molecules that contain one or more SNPs disclosed in Tables 1-433. Table 1 provides context nucleic acid sequences. Tables 3-433 provide only rs identification numbers; however, the context sequences for such SNPs are known and disclosed in the art, and are not therefore shown in the tables. Isolated nucleic acid molecules contain one or more SNPs identified in Tables 1-433. Isolated nucleic acid molecules containing one or more SNPs disclosed in Tables 1-433 may be interchangeably referred to throughout the present text as “SNP-containing nucleic acid molecules.” The isolated nucleic acid molecules of the present invention also include probes and primers (which are described in greater detail below in the section entitled “SNP Detection Reagents”), which may be used for assaying the disclosed SNPs, and isolated full-length genes, transcripts, cDNA molecules, and fragments thereof, which may be used for such purposes as expressing an encoded protein.

As used herein, an “isolated nucleic acid molecule” generally is one that contains a SNP of the present invention or one that hybridizes to such molecule such as a nucleic acid with a complementary sequence, and is separated from most other nucleic acids present in the natural source of the nucleic acid molecule. Moreover, an “isolated” nucleic acid molecule, such as a cDNA molecule containing a SNP of the present invention, can be substantially free of other cellular material, or culture medium when produced by recombinant techniques, or chemical precursors or other chemicals when chemically synthesized. A nucleic acid molecule can be fused to other coding or regulatory sequences and still be considered “isolated.” Nucleic acid molecules present in non-human transgenic animals, which do not naturally occur in the animal, are also considered “isolated”. For example, recombinant DNA molecules contained in a vector are considered “isolated”. Further examples of “isolated” DNA molecules include recombinant DNA molecules maintained in heterologous host cells, and purified (partially or substantially) DNA molecules in solution. Isolated RNA molecules include in vivo or in vitro RNA transcripts of the isolated SNP-containing DNA molecules of the present invention. Isolated nucleic acid molecules according to the present invention further include such molecules produced synthetically.

Generally, an isolated SNP-containing nucleic acid molecule comprises one or more SNP positions disclosed by the present invention with flanking nucleotide sequences on either side of the SNP positions. A flanking sequence can include nucleotide residues that are naturally associated with the SNP site and/or heterologous nucleotide sequences. The flanking sequence may be up to about 100, 60, 50, 30, 25, 20, 15, 10, 8, or 4 nucleotides (or any other length in-between) on either side of a SNP position.

For full-length genes and entire protein-coding sequences, a SNP flanking sequence can be, for example, up to, but not limited to, about 5 KB, 4 KB, 3 KB, 2 KB, 1 KB on either side of the SNP. Furthermore, in such instances, the isolated nucleic acid molecule comprises exonic sequences (including protein-coding and/or non-coding exonic sequences), but may also include intronic sequences. Thus, any protein coding sequence may be either contiguous or separated by introns. The important point is that the nucleic acid is isolated from remote and unimportant flanking sequences and is of appropriate length such that it can be subjected to the specific manipulations or uses described herein such as recombinant protein expression, preparation of probes and primers for assaying the SNP position, and other uses specific to the SNP-containing nucleic acid sequences.

An isolated SNP-containing nucleic acid molecule can comprise, for example, a full-length gene or transcript, such as a gene isolated from genomic DNA (e.g., by cloning or PCR amplification), a cDNA molecule, or an mRNA transcript molecule. Furthermore, fragments of such full-length genes and transcripts that contain one or more SNPs disclosed herein are also encompassed by the present invention, and such fragments may be used, for example, to express any part of a protein, such as a particular functional domain or an antigenic epitope.

Thus, the present invention also encompasses fragments of the nucleic acid sequences contiguous to the SNPs disclosed in Tables 1-433, contiguous nucleotide sequence at least about 8 or more nucleotides, more preferably at least about 12 or more nucleotides, and even more preferably at least about 16 or more nucleotides. Further, a fragment could comprise at least about 18, 20, 22, 25, 30, 40, 50, 60, 100, 250 or 500 (or any other number in-between) nucleotides in length. The length of the fragment will be based on its intended use. For example, the fragment can be useful as a polynucleotide probe or primer. Such fragments can be isolated using nucleotide sequences comprising one of the SNPs in Tables 1-433 for the synthesis of a polynucleotide probe. A labeled probe can then be used, for example, to screen a cDNA library, genomic DNA library, or mRNA to isolate nucleic acid corresponding to the coding region. Further, primers can be used in amplification reactions, such as for purposes of assaying one or more SNP sites or for cloning specific regions of a gene.

An isolated nucleic acid molecule of the present invention further encompasses a SNP-containing polynucleotide that is the product of any one of a variety of nucleic acid amplification methods, which are used to increase the copy numbers of a polynucleotide of interest in a nucleic acid sample. Such amplification methods are well known in the art, and they include but are not limited to, polymerase chain reaction (PCR) (U.S. Pat. Nos. 4,683,195; and 4,683,202; PCR Technology: Principles and Applications for DNA Amplification, ed. H. A. Erlich, Freeman Press, NY, N.Y., 1992), ligase chain reaction (LCR) (Wu and Wallace, Genomics 4:560, 1989; Landegren et al., Science 241:1077, 1988), strand displacement amplification (SDA) (U.S. Pat. Nos. 5,270,184; and 5,422,252), transcription-mediated amplification (TMA) (U.S. Pat. No. 5,399,491), linked linear amplification (LLA) (U.S. Pat. No. 6,027,923), and the like, and isothermal amplification methods such as nucleic acid sequence based amplification (NASBA), and self-sustained sequence replication (Guatelli et al., Proc. Natl. Acad. Sci. USA 87: 1874, 1990). Based on such methodologies, a person skilled in the art can readily design primers in any suitable regions 5′ and 3′ to a SNP disclosed herein. Such primers may be used to amplify DNA of any length so long that it contains the SNP of interest in its sequence.

As used herein, an “amplified polynucleotide” of the invention is a SNP-containing nucleic acid molecule whose amount has been increased at least two fold by any nucleic acid amplification method performed in vitro as compared to its starting amount in a test sample. In other preferred embodiments, an amplified polynucleotide is the result of at least ten fold, fifty fold, one hundred fold, one thousand fold, or even ten thousand fold increase as compared to its starting amount in a test sample. In a typical PCR amplification, a polynucleotide of interest is often amplified at least fifty thousand fold in amount over the unamplified genomic DNA, but the precise amount of amplification needed for an assay depends on the sensitivity of the subsequent detection method used.

Generally, an amplified polynucleotide is at least about 16 nucleotides in length. More typically, an amplified polynucleotide is at least about 20 nucleotides in length. In a preferred embodiment of the invention, an amplified polynucleotide is at least about 30 nucleotides in length. In a more preferred embodiment of the invention, an amplified polynucleotide is at least about 32, 40, 45, 50, or 60 nucleotides in length. In yet another preferred embodiment of the invention, an amplified polynucleotide is at least about 100, 200, or 300 nucleotides in length. While the total length of an amplified polynucleotide of the invention can be as long as an exon, an intron or the entire gene where the SNP of interest resides, an amplified product is typically no greater than about 1,000 nucleotides in length (although certain amplification methods may generate amplified products greater than 1000 nucleotides in length). More preferably, an amplified polynucleotide is not greater than about 600 nucleotides in length. It is understood that irrespective of the length of an amplified polynucleotide, a SNP of interest may be located anywhere along its sequence.

In a specific embodiment of the invention, the amplified product is at least about 201 nucleotides in length, comprises one of the nucleotide sequences shown in Tables 1-433. Such a product may have additional sequences on its 5′ end or 3′ end or both. In another embodiment, the amplified product is about 101 nucleotides in length, and it contains a SNP disclosed herein. Generally, the SNP is located at the middle of the amplified product (e.g., at position 101 in an amplified product that is 201 nucleotides in length, or at position 51 in an amplified product that is 101 nucleotides in length), or within 1, 2, 3, 4, 5, 6, 7, 8, 9, 10, 12, 15, or 20 nucleotides from the middle of the amplified product (however, as indicated above, the SNP of interest may be located anywhere along the length of the amplified product).

The present invention provides isolated nucleic acid molecules that comprise, consist of, or consist essentially of one or more SNPs disclosed herein, complements thereof, and SNP-containing fragments thereof.

Accordingly, the present invention provides nucleic acid molecules that consist of any of the nucleotide sequences comprising one of the SNPs shown in Tables 1-433. A nucleic acid molecule consists of a nucleotide sequence when the nucleotide sequence is the complete nucleotide sequence of the nucleic acid molecule.

The present invention further provides nucleic acid molecules that consist essentially of any of the SNPs shown in Tables 1-433. A nucleic acid molecule consists essentially of a nucleotide sequence when such a nucleotide sequence includes only one of the SNPs disclosed in Tables 1-433, and no other SNPs associated with endometriosis, although additional nucleotide sequence may be included that does not include any additional SNPs associated with endometriosis.

The present invention further provides nucleic acid molecules that comprise any of the SNPs shown in Tables 1-433. A nucleic acid molecule comprises a nucleotide sequence when the nucleotide sequence is at least part of the final nucleotide sequence of the nucleic acid molecule. In such a fashion, the nucleic acid molecule can be only the nucleotide sequence or have additional nucleotide residues, such as residues that are naturally associated with it or heterologous nucleotide sequences. Such a nucleic acid molecule can have one to a few additional nucleotides or can comprise many more additional nucleotides. A brief description of how various types of these nucleic acid molecules can be readily made and isolated are well known to those of ordinary skill in the art (Sambrook and Russell, 2000, Molecular Cloning: A Laboratory Manual, Third ed. Woodbury, NY: CSHL Press; 2001).

Isolated nucleic acid molecules can be in the form of RNA, such as mRNA, or in the form DNA, including cDNA and genomic DNA, which may be obtained, for example, by molecular cloning or produced by chemical synthetic techniques or by a combination thereof (Sambrook and Russell, Molecular Cloning: A Laboratory Manual, Third ed. Woodbury, NY: CSHL Press; 2001). Furthermore, isolated nucleic acid molecules, particularly SNP detection reagents such as probes and primers, can also be partially or completely in the form of one or more types of nucleic acid analogs, such as peptide nucleic acid (PNA) (U.S. Pat. Nos. 5,539,082; 5,527,675; 5,623,049; 5,714,331). The nucleic acid, especially DNA, can be double-stranded or single-stranded. Single-stranded nucleic acid can be the coding strand (sense strand) or the complementary non-coding strand (anti-sense strand). DNA, RNA, or PNA segments can be assembled, for example, from fragments of the human genome (in the case of DNA or RNA) or single nucleotides, short oligonucleotide linkers, or from a series of oligonucleotides, to provide a synthetic nucleic acid molecule. Nucleic acid molecules can be readily synthesized using the sequences provided herein as a reference; oligonucleotide and PNA oligomer synthesis techniques are well known in the art (see, Corey D R. Peptide nucleic acids: expanding the scope of nucleic acid recognition. Trends Biotechnol. June 1997; 15(6):224-229 and Hyrup B, Nielsen P E. Peptide nucleic acids (PNA): synthesis, properties and potential applications. Bioorg Med Chem. January 1996; 4(1):5-23).

The present invention encompasses nucleic acid analogs that contain modified, synthetic, or non-naturally occurring nucleotides or structural elements or other alternative/modified nucleic acid chemistries known in the art. Such nucleic acid analogs are useful, for example, as detection reagents (e.g., primers/probes) for detecting one or more SNPs identified in Tables 1-433. Furthermore, kits/systems (such as beads, arrays, etc.) that include these analogs are also encompassed by the present invention.

Additional examples of nucleic acid modifications that improve the binding properties and/or stability of a nucleic acid include the use of base analogs such as inosine, intercalators (U.S. Pat. No. 4,835,263) and the minor groove binders (U.S. Pat. No. 5,801,115). Thus, references herein to nucleic acid molecules, SNP-containing nucleic acid molecules, SNP detection reagents (e.g., probes and primers), oligonucleotides/polynucleotides include PNA oligomers and other nucleic acid analogs. Other examples of nucleic acid analogs and alternative/modified nucleic acid chemistries known in the art are described in Beaucage et al. (Current Protocols in Nucleic Acid Chemistry. New York: John Wiley and Sons; 2007).

Further variants of the SNPs disclosed in Tables 1-433, such as naturally occurring allelic variants (as well as orthologs and paralogs) and synthetic variants produced by mutagenesis techniques, can be identified and/or produced using methods well known in the art. Such further variants can comprise a nucleotide sequence that shares at least 70-80%, 80-85%, 85-90%, 91%, 92%, 93%, 94%, 95%, 96%, 97%, 98%, or 99% sequence identity with a nucleic acid sequence contiguous to the SNPs disclosed in Tables 1-433 (or a fragment thereof) and that includes a novel SNP allele disclosed in Tables 1-433. Thus, the present invention specifically contemplates isolated nucleic acid molecule that have a certain degree of sequence variation compared with the sequences shown in Tables 1-433, but that contain a novel SNP allele disclosed herein. In other words, as long as an isolated nucleic acid molecule contains a novel SNP allele disclosed herein, other portions of the nucleic acid molecule that flank the novel SNP allele can vary to some degree from the specific genomic and context sequences surrounding the SNPs listed in Tables 1-433.

The present invention further provides non-coding fragments of the nucleic acid molecules disclosed in Tables 1-433. Preferred non-coding fragments include, but are not limited to, promoter sequences, enhancer sequences, intronic sequences, 5′ untranslated regions (UTRs), 3′ untranslated regions, gene modulating sequences and gene termination sequences. Such fragments are useful, for example, in controlling heterologous gene expression and in developing screens to identify gene-modulating agents.

SNP Detection Reagents

In a specific aspect of the present invention, the SNPs disclosed herein can be used for the design of SNP detection reagents. As used herein, a “SNP detection reagent” is a reagent that specifically detects a specific target SNP position disclosed herein, and that is preferably specific for a particular nucleotide (allele) of the target SNP position (i.e., the detection reagent preferably can differentiate between different alternative nucleotides at a target SNP position, thereby allowing the identity of the nucleotide present at the target SNP position to be determined). Typically, such detection reagent hybridizes to a target SNP-containing nucleic acid molecule by complementary base-pairing in a sequence specific manner, and discriminates the target variant sequence from other nucleic acid sequences such as an art-known form in a test sample. An example of a detection reagent is a probe that hybridizes to a target nucleic acid containing one or more of the SNPs disclosed herein. In a preferred embodiment, such a probe can differentiate between nucleic acids having a particular nucleotide (allele) at a target SNP position from other nucleic acids that have a different nucleotide at the same target SNP position. In addition, a detection reagent may hybridize to a specific region 5′ and/or 3′ to a SNP position, particularly a region corresponding to the context sequences provided in the SNPs disclosed herein. Another example of a detection reagent is a primer which acts as an initiation point of nucleotide extension along a complementary strand of a target polynucleotide. The SNP sequence information provided herein is also useful for designing primers, e.g. allele-specific primers, to amplify (e.g., using PCR) any SNP of the present invention.

In one preferred embodiment of the invention, a SNP detection reagent is a synthetic polynucleotide molecule, such as an isolated or synthetic DNA or RNA polynucleotide probe or primer or PNA oligomer, or a combination of DNA, RNA and/or PNA that hybridizes to a segment of a target nucleic acid molecule containing a SNP identified herein. A detection reagent in the form of a polynucleotide may optionally contain modified base analogs, intercalators or minor groove binders. Multiple detection reagents such as probes may be, for example, affixed to a solid support (e.g., arrays or beads) or supplied in solution (e.g., probe/primer sets for enzymatic reactions such as PCR, RT-PCR, TaqMan assays, or primer-extension reactions) to form a SNP detection kit.

A probe or primer typically is a substantially purified oligonucleotide. Such oligonucleotide typically comprises a region of complementary nucleotide sequence that hybridizes under stringent conditions to at least about 8, 10, 12, 16, 18, 20, 22, 25, 30, 40, 50, 60, 100 (or any other number in-between) or more consecutive nucleotides in a target nucleic acid molecule. Depending on the particular assay, the consecutive nucleotides can either include the target SNP position, or be a specific region in close enough proximity 5′ and/or 3′ to the SNP position to carry out the desired assay.

Other preferred primer and probe sequences can readily be determined using the nucleotide sequences disclosed herein. It will be apparent to one of skill in the art that such primers and probes are directly useful as reagents for genotyping the SNPs of the present invention, and can be incorporated into any kit/system format.

In order to produce a probe or primer specific for a target SNP-containing sequence, the gene/transcript and/or context sequence surrounding the SNP of interest is typically examined using a computer algorithm which starts at the 5′ or at the 3′ end of the nucleotide sequence. Typical algorithms will then identify oligomers of defined length that are unique to the gene/SNP context sequence, have a GC content within a range suitable for hybridization, lack predicted secondary structure that may interfere with hybridization, and/or possess other desired characteristics or that lack other undesired characteristics.

A primer or probe of the present invention is typically at least about 8 nucleotides in length. In one embodiment of the invention, a primer or a probe is at least about 10 nucleotides in length. In a preferred embodiment, a primer or a probe is at least about 12 nucleotides in length. In a more preferred embodiment, a primer or probe is at least about 16, 17, 18, 19, 20, 21, 22, 23, 24 or 25 nucleotides in length. While the maximal length of a probe can be as long as the target sequence to be detected, depending on the type of assay in which it is employed, it is typically less than about 50, 60, 65, or 70 nucleotides in length. In the case of a primer, it is typically less than about 30 nucleotides in length. In a specific preferred embodiment of the invention, a primer or a probe is within the length of about 18 and about 28 nucleotides. However, in other embodiments, such as nucleic acid arrays and other embodiments in which probes are affixed to a substrate, the probes can be longer, such as on the order of 30-70, 75, 80, 90, 100, or more nucleotides in length (see the section below entitled “SNP Detection Kits and Systems”).

For analyzing SNPs, it may be appropriate to use oligonucleotides specific for alternative SNP alleles. Such oligonucleotides which detect single nucleotide variations in target sequences may be referred to by such terms as “allele-specific oligonucleotides”, “allele-specific probes”, or “allele-specific primers”. The design and use of allele-specific probes for analyzing polymorphisms is described in, e.g., Cotton R G H, et al., eds. Mutation Detection: A Practical Approach. New York: Oxford University Press; 1998. Harnes B D, ed. The Practical Approach Series and Saiki R K, et al. Analysis of enzymatically amplified beta-globin and HLA-DQ alpha DNA with allele-specific oligonucleotide probes. Nature. Nov. 13-19, 1986; 324(6093):163-166.

While the design of each allele-specific primer or probe depends on variables such as the precise composition of the nucleotide sequences flanking a SNP position in a target nucleic acid molecule, and the length of the primer or probe, another factor in the use of primers and probes is the stringency of the condition under which the hybridization between the probe or primer and the target sequence is performed. Higher stringency conditions utilize buffers with lower ionic strength and/or a higher reaction temperature, and tend to require a more perfect match between probe/primer and a target sequence in order to form a stable duplex. If the stringency is too high, however, hybridization may not occur at all. In contrast, lower stringency conditions utilize buffers with higher ionic strength and/or a lower reaction temperature, and permit the formation of stable duplexes with more mismatched bases between a probe/primer and a target sequence. By way of example and not limitation, exemplary conditions for high stringency hybridization conditions using an allele-specific probe are as follows: Prehybridization with a solution containing 5× standard saline phosphate EDTA (SSPE), 0.5% NaDodSO4 (SDS) at 55° C., and incubating probe with target nucleic acid molecules in the same solution at the same temperature, followed by washing with a solution containing 2×SSPE, and 0.1% SDS at 55° C. or room temperature.

Moderate stringency hybridization conditions may be used for allele-specific primer extension reactions with a solution containing, e.g., about 50 mM KCl at about 46° C. Alternatively, the reaction may be carried out at an elevated temperature such as 60° C. In another embodiment, a moderately stringent hybridization condition suitable for oligonucleotide ligation assay (OLA) reactions wherein two probes are ligated if they are completely complementary to the target sequence may utilize a solution of about 100 mM KCl at a temperature of 46° C.

In a hybridization-based assay, allele-specific probes can be designed that hybridize to a segment of target DNA from one individual but do not hybridize to the corresponding segment from another individual due to the presence of different polymorphic forms (e.g., alternative SNP alleles/nucleotides) in the respective DNA segments from the two individuals. Hybridization conditions should be sufficiently stringent that there is a significant detectable difference in hybridization intensity between alleles, and preferably an essentially binary response, whereby a probe hybridizes to only one of the alleles or significantly more strongly to one allele. While a probe may be designed to hybridize to a target sequence that contains a SNP site such that the SNP site aligns anywhere along the sequence of the probe, the probe is preferably designed to hybridize to a segment of the target sequence such that the SNP site aligns with a central position of the probe (e.g., a position within the probe that is at least three nucleotides from either end of the probe). This design of probe generally achieves good discrimination in hybridization between different allelic forms.

In another embodiment, a probe or primer may be designed to hybridize to a segment of target DNA such that the SNP aligns with either the 5′ most end or the 3′ most end of the probe or primer. In a specific preferred embodiment which is particularly suitable for use in a oligonucleotide ligation assay (U.S. Pat. No. 4,988,617), the most 3′nucleotide of the probe aligns with the SNP position in the target sequence.

Oligonucleotide probes and primers may be prepared by methods well known in the art. Chemical synthetic methods include, but are not limited to, the phosphotriester method described by Narang et al. (Improved phosphotriester method for the synthesis of gene fragments. Methods Enzymol. 1979; 68:90-98); the phosphodiester method described by Brown et al., (Chemical synthesis and cloning of a tyrosine tRNA gene. Methods Enzymol. 1979; 68:109-151), the diethylphosphoamidate method described by Beaucage and Caruthers (Deoxynucleoside phosphoramidites—A new class of key intermediates for deoxypolynucleotide synthesis. Tetrahedron Letters. 1981; 22(20):1859-1862); and the solid support method described in U.S. Pat. No. 4,458,066.

Allele-specific probes are often used in pairs (or, less commonly, in sets of 3 or 4, such as if a SNP position is known to have 3 or 4 alleles, respectively, or to assay both strands of a nucleic acid molecule for a target SNP allele), and such pairs may be identical except for a one nucleotide mismatch that represents the allelic variants at the SNP position. Commonly, one member of a pair perfectly matches a reference form of a target sequence that has a more common SNP allele (i.e., the allele that is more frequent in the target population) and the other member of the pair perfectly matches a form of the target sequence that has a less common SNP allele (i.e., the allele that is rarer in the target population). In the case of an array, multiple pairs of probes can be immobilized on the same support for simultaneous analysis of multiple different polymorphisms.

In one type of PCR-based assay, an allele-specific primer hybridizes to a region on a target nucleic acid molecule that overlaps a SNP position and only primes amplification of one allelic form to which the primer exhibits perfect complementarity (Gibbs R A, et al. Detection of single DNA base differences by competitive oligonucleotide priming. Nucleic Acids Res. Apr. 11, 1989; 17(7):2437-2448). Typically, the primer's 3′-most nucleotide is aligned with and complementary to the SNP position of the target nucleic acid molecule. This primer is used in conjunction with a second primer that hybridizes at a distal site. Amplification proceeds from the two primers, producing a detectable product that indicates which allelic form is present in the test sample. A control is usually performed with a second pair of primers, one of which shows a single base mismatch at the polymorphic site and the other of which exhibits perfect complementarity to a distal site. The single-base mismatch prevents amplification or substantially reduces amplification efficiency, so that either no detectable product is formed or it is formed in lower amounts or at a slower pace. The method generally works most effectively when the mismatch is at the 3′-most position of the oligonucleotide (i.e., the 3′-most position of the oligonucleotide aligns with the target SNP position) because this position is most destabilizing to elongation from the primer (see, e.g., WIPO patent WO/1993/022456). This PCR-based assay can be utilized as part of the TaqMan assay, described below.

In a specific embodiment of the invention, a primer of the invention contains a sequence substantially complementary to a segment of a target SNP-containing nucleic acid molecule except that the primer has a mismatched nucleotide in one of the three nucleotide positions at the 3′-most end of the primer, such that the mismatched nucleotide does not base pair with a particular allele at the SNP site. In a preferred embodiment, the mismatched nucleotide in the primer is the second from the last nucleotide at the 3′-most position of the primer. In a more preferred embodiment, the mismatched nucleotide in the primer is the last nucleotide at the 3′-most position of the primer.

In another embodiment of the invention, a SNP detection reagent of the invention is labeled with a fluorogenic reporter dye that emits a detectable signal. While the preferred reporter dye is a fluorescent dye, any reporter dye that can be attached to a detection reagent such as an oligonucleotide probe or primer is suitable for use in the invention. Such dyes include, but are not limited to, Acridine, AMCA, BODIPY, Cascade Blue, Cy2, Cy3, Cy5, Cy7, Dabcyl, Edans, Eosin, Erythrosin, Fluorescein, 6-Fam, Tet, Joe, Hex, Oregon Green, Rhodamine, Rhodol Green, Tamra, Rox, and Texas Red.

In yet another embodiment of the invention, the detection reagent may be further labeled with a quencher dye such as Tamra, especially when the reagent is used as a self-quenching probe such as a TaqMan (U.S. Pat. Nos. 5,210,015 and 5,538,848) or Molecular Beacon probe (U.S. Pat. Nos. 5,118,801 and 5,312,728), or other stemless or linear beacon probe (Livak K J, et al. Oligonucleotides with fluorescent dyes at opposite ends provide a quenched probe system useful for detecting PCR product and nucleic acid hybridization. PCR Methods Appl. June 1995; 4(6):357-362. Tyagi S, Kramer F R. Molecular beacons: probes that fluoresce upon hybridization. Nat Biotechnol. March 1996; 14(3):303-308. Nazarenko I A, et al. A closed tube format for amplification and detection of DNA based on energy transfer. Nucleic Acids Res. Jun. 15, 1997; 25(12):2516-2521).

The detection reagents of the invention may also contain other labels, including but not limited to, biotin for streptavidin binding and oligonucleotide for binding to another complementary oligonucleotide such as pairs of zipcodes.

The present invention also contemplates reagents that do not contain (or that are complementary to) a SNP nucleotide identified herein but that are used to assay one or more SNPs disclosed herein. For example, primers that flank, but do not hybridize directly to a target SNP position provided herein are useful in primer extension reactions in which the primers hybridize to a region adjacent to the target SNP position (i.e., within one or more nucleotides from the target SNP site). During the primer extension reaction, a primer is typically not able to extend past a target SNP site if a particular nucleotide (allele) is present at that target SNP site, and the primer extension product can readily be detected in order to determine which SNP allele is present at the target SNP site. For example, particular ddNTPs are typically used in the primer extension reaction to terminate primer extension once a ddNTP is incorporated into the extension product (a primer extension product which includes a ddNTP at the 3′-most end of the primer extension product, and in which the ddNTP corresponds to a SNP disclosed herein, is a composition that is encompassed by the present invention). Thus, reagents that bind to a nucleic acid molecule in a region adjacent to a SNP site, even though the bound sequences do not necessarily include the SNP site itself, are also encompassed by the present invention.

SNP Detection Kits and Systems

A person skilled in the art will recognize that, based on the SNP and associated sequence information disclosed herein, detection reagents can be developed and used to assay any SNP of the present invention individually or in combination, and such detection reagents can be readily incorporated into one of the established kit or system formats which are well known in the art. The terms “kits” and “systems”, as used herein in the context of SNP detection reagents, are intended to refer to such things as combinations of multiple SNP detection reagents, or one or more SNP detection reagents in combination with one or more other types of elements or components (e.g., other types of biochemical reagents, containers, packages such as packaging intended for commercial sale, substrates to which SNP detection reagents are attached, electronic hardware components, etc.). Accordingly, the present invention further provides SNP detection kits and systems, including but not limited to, packaged probe and primer sets (e.g., TaqMan probe/primer sets), arrays/microarrays of nucleic acid molecules, and beads that contain one or more probes, primers, or other detection reagents for detecting one or more SNPs of the present invention. The kits/systems can optionally include various electronic hardware components; for example, arrays (“DNA chips”) and microfluidic systems (“lab-on-a-chip” systems) provided by various manufacturers typically comprise hardware components. Other kits/systems (e.g., probe/primer sets) may not include electronic hardware components, but may be comprised of, for example, one or more SNP detection reagents (along with, optionally, other biochemical reagents) packaged in one or more containers.

In some embodiments, a SNP detection kit typically contains one or more detection reagents and other components (e.g., a buffer, enzymes such as DNA polymerases or ligases, chain extension nucleotides such as deoxynucleotide triphosphates, and in the case of Sanger-type DNA sequencing reactions, chain terminating nucleotides, positive control sequences, negative control sequences, and the like) necessary to carry out an assay or reaction, such as amplification and/or detection of a SNP-containing nucleic acid molecule. A kit may further contain means for determining the amount of a target nucleic acid, and means for comparing the amount with a standard, and can comprise instructions for using the kit to detect the SNP-containing nucleic acid molecule of interest. In one embodiment of the present invention, kits are provided which contain the necessary reagents to carry out one or more assays to detect one or more SNPs disclosed herein. In a preferred embodiment of the present invention, SNP detection kits/systems are in the form of nucleic acid arrays, or compartmentalized kits, including microfluidic/lab-on-a-chip systems.

SNP detection kits/systems may contain, for example, one or more probes, or pairs of probes, that hybridize to a nucleic acid molecule at or near each target SNP position. Multiple pairs of allele-specific probes may be included in the kit/system to simultaneously assay large numbers of SNPs, at least one of which is a SNP of the present invention. In some kits/systems, the allele-specific probes are immobilized to a substrate such as an array or bead. For example, the same substrate can comprise allele-specific probes for detecting at least 1; 10; 100; 1000; 10,000; 100,000; 500,000 (or any other number in-between) or substantially all of the SNPs disclosed herein.

The terms “arrays,” “microarrays,” and “DNA chips” are used herein interchangeably to refer to an array of distinct polynucleotides affixed to a substrate, such as glass, plastic, paper, nylon or other type of membrane, filter, chip, or any other suitable solid support. The polynucleotides can be synthesized directly on the substrate, or synthesized separate from the substrate and then affixed to the substrate. In one embodiment, the microarray is prepared and used according to the methods described in U.S. Pat. No. 5,837,832 (to Chee et al.), PCT application WO1995/011995 (to Chee et al.), Lockhart et al. (Expression monitoring by hybridization to high-density oligonucleotide arrays. Nat Biotechnol. December 1996; 14(13):1675-1680); and Schena et al. (Parallel human genome analysis: microarray-based expression monitoring of 1000 genes. Proc Natl Acad Sci USA. Oct. 1, 1996; 93(20):10614-10619), all of which are incorporated herein in their entirety by reference. In other embodiments, such arrays are produced by the methods described in U.S. Pat. No. 5,807,522 (to Brown et al.).

Nucleic acid arrays are reviewed in the following references: Zammatteo et al. (New chips for molecular biology and diagnostics. Biotechnol Annu Rev. 2002; 8:85-101); Sosnowski et al. (Active microelectronic array system for DNA hybridization, genotyping and pharmacogenomic applications. Psychiatr Genet. December 2002; 12(4):181-192); Heller (DNA microarray technology: devices, systems, and applications Annu Rev Biomed Eng. 2002; 4:129-153); Kolchinsky and Mirzabekov (Analysis of SNPs and other genomic variations using gel-based chips. Hum Mutat. April 2002; 19(4):343-360); and McGall and Christians (High-density genechip oligonucleotide probe arrays. Adv Biochem Eng Biotechnol. 2002; 77:21-42).

Any number of probes, such as allele-specific probes, may be implemented in an array, and each probe or pair of probes can hybridize to a different SNP position. In the case of polynucleotide probes, they can be synthesized at designated areas (or synthesized separately and then affixed to designated areas) on a substrate using a light-directed chemical process. Each DNA chip can contain, for example, thousands to millions of individual synthetic polynucleotide probes arranged in a grid-like pattern and miniaturized (e.g., to the size of a dime). Preferably, probes are attached to a solid support in an ordered, addressable array.

A microarray can be composed of a large number of unique, single-stranded polynucleotides fixed to a solid support. Typical polynucleotides are preferably about 6-60 nucleotides in length, more preferably about 15-30 nucleotides in length, and most preferably about 18-25 nucleotides in length. For certain types of microarrays or other detection kits/systems, it may be preferable to use oligonucleotides that are only about 7-20 nucleotides in length. In other types of arrays, such as arrays used in conjunction with chemiluminescent detection technology, preferred probe lengths can be, for example, about 15-80 nucleotides in length, preferably about 50-70 nucleotides in length, more preferably about 55-65 nucleotides in length, and most preferably about 60 nucleotides in length. The microarray or detection kit can contain polynucleotides that cover the known 5′ or 3′ sequence of the target SNP site, sequential polynucleotides that cover the full-length sequence of a gene/transcript; or unique polynucleotides selected from particular areas along the length of a target gene/transcript sequence, particularly areas corresponding to one or more SNPs disclosed herein. Polynucleotides used in the microarray or detection kit can be specific to a SNP or SNPs of interest (e.g., specific to a particular SNP allele at a target SNP site, or specific to particular SNP alleles at multiple different SNP sites), or specific to a polymorphic gene/transcript or genes/transcripts of interest.

Hybridization assays based on polynucleotide arrays rely on the differences in hybridization stability of the probes to perfectly matched and mismatched target sequence variants. For SNP genotyping, it is generally preferable that stringency conditions used in hybridization assays are high enough such that nucleic acid molecules that differ from one another at as little as a single SNP position can be differentiated (e.g., typical SNP hybridization assays are designed so that hybridization will occur only if one particular nucleotide is present at a SNP position, but will not occur if an alternative nucleotide is present at that SNP position). Such high stringency conditions may be preferable when using, for example, nucleic acid arrays of allele-specific probes for SNP detection. Such high stringency conditions are described in the preceding section, and are well known to those skilled in the art and can be found in, for example, Ausubel et al. (Current Protocols in Molecular Biology. New York: John Wiley and Sons; 2007).

In other embodiments, the arrays are used in conjunction with chemiluminescent detection technology. The following patents and patent applications, which are all hereby incorporated by reference, provide additional information pertaining to chemiluminescent detection: U.S. patent application Ser. Nos. 10/620,332 and 10/620,333 describe chemiluminescent approaches for microarray detection; U.S. Pat. Nos. 6,124,478, 6,107,024, 5,994,073, 5,981,768, 5,871,958, 5,843,681, 5,800,999, and 5,773,628 describe methods and compositions of dioxetane for performing chemiluminescent detection; and U.S. published application US2002/0110828 discloses methods and compositions for microarray controls.

In one embodiment of the invention, a nucleic acid array can comprise an array of probes of about 15-25 nucleotides in length. In a further embodiment, a nucleic acid array can comprise an array of probes of about 15-75 nucleotides in length. In yet further embodiments, a nucleic acid array can comprise any number of probes, in which at least one probe is capable of detecting one or more SNPs disclosed in Table 1 and/or at least one probe comprises a fragment of one of the sequences selected from the group consisting of those disclosed herein, and sequences complementary thereto, said fragment comprising at least about 8 consecutive nucleotides, preferably 10, 12, 15, 16, 18, 20, more preferably 22, 25, 30, 40, 47, 50, 55, 60, 65, 70, 80, 90, 100, or more consecutive nucleotides (or any other number in-between) and containing (or being complementary to) a SNP. In some embodiments, the nucleotide complementary to the SNP site is within 5, 4, 3, 2, or 1 nucleotide from the center of the probe, more preferably at the center of said probe.

A polynucleotide probe can be synthesized on the surface of the substrate by using a chemical coupling procedure and an ink jet application apparatus, as described in PCT application WO95/251116 (to Baldeschweiler et al.) which is incorporated herein in its entirety by reference. In another aspect, a “gridded” array analogous to a dot (or slot) blot may be used to arrange and link cDNA fragments or oligonucleotides to the surface of a substrate using a vacuum system, thermal, UV, mechanical or chemical bonding procedures. An array, such as those described above, may be produced by hand or by using available devices (slot blot or dot blot apparatus), materials (any suitable solid support), and machines (including robotic instruments), and may contain 8, 24, 96, 384, 1536, 6144 or more polynucleotides, or any other number which lends itself to the efficient use of commercially available instrumentation.

Using such arrays or other kits/systems, the present invention provides methods of identifying the SNPs disclosed herein in a test sample. Such methods typically involve incubating a test sample of nucleic acids with an array comprising one or more probes corresponding to at least one SNP position of the present invention, and assaying for binding of a nucleic acid from the test sample with one or more of the probes. Conditions for incubating a SNP detection reagent (or a kit/system that employs one or more such SNP detection reagents) with a test sample vary. Incubation conditions depend on such factors as the format employed in the assay, the detection methods employed, and the type and nature of the detection reagents used in the assay. One skilled in the art will recognize that any one of the commonly available hybridization, amplification and array assay formats can readily be adapted to detect the SNPs disclosed herein.

A SNP detection kit/system of the present invention may include components that are used to prepare nucleic acids from a test sample for the subsequent amplification and/or detection of a SNP-containing nucleic acid molecule. Such sample preparation components can be used to produce nucleic acid extracts, including DNA and/or RNA, extracts from any bodily fluids. In a preferred embodiment of the invention, the bodily fluid is blood, saliva or buccal swabs. The test samples used in the above-described methods will vary based on such factors as the assay format, nature of the detection method, and the specific tissues, cells or extracts used as the test sample to be assayed. Methods of preparing nucleic acids are well known in the art and can be readily adapted to obtain a sample that is compatible with the system utilized.

In yet another form of the kit in addition to reagents for preparation of nucleic acids and reagents for detection of one of the SNPs of this invention, the kit may include a questionnaire inquiring about non-genetic clinical factors such as age, gender, or any other non-genetic clinical factors known to be associated with endometriosis.

Another form of kit contemplated by the present invention is a compartmentalized kit. A compartmentalized kit includes any kit in which reagents are contained in separate containers. Such containers include, for example, small glass containers, plastic containers, strips of plastic, glass or paper, or arraying material such as silica. Such containers allow one to efficiently transfer reagents from one compartment to another compartment such that the test samples and reagents are not cross-contaminated, or from one container to another vessel not included in the kit, and the agents or solutions of each container can be added in a quantitative fashion from one compartment to another or to another vessel. Such containers may include, for example, one or more containers which will accept the test sample, one or more containers which contain at least one probe or other SNP detection reagent for detecting one or more SNPs of the present invention, one or more containers which contain wash reagents (such as phosphate buffered saline, Tris-buffers, etc.), and one or more containers which contain the reagents used to reveal the presence of the bound probe or other SNP detection reagents. The kit can optionally further comprise compartments and/or reagents for, for example, nucleic acid amplification or other enzymatic reactions such as primer extension reactions, hybridization, ligation, electrophoresis (preferably capillary electrophoresis), mass spectrometry, and/or laser-induced fluorescent detection. The kit may also include instructions for using the kit. Exemplary compartmentalized kits include microfluidic devices known in the art (see, e.g., Weigl B H, et al. Lab-on-a-chip for drug development. Adv Drug Deliv Rev. Feb. 24, 2003; 55(3):349-377). In such microfluidic devices, the containers may be referred to as, for example, microfluidic “compartments”, “chambers”, or “channels”.

Microfluidic devices, which may also be referred to as “lab-on-a-chip” systems, biomedical micro-electro-mechanical systems (bioMEMs), or multicomponent integrated systems, are exemplary kits/systems of the present invention for analyzing SNPs. Such systems miniaturize and compartmentalize processes such as probe/target hybridization, nucleic acid amplification, and capillary electrophoresis reactions in a single functional device. Such microfluidic devices typically utilize detection reagents in at least one aspect of the system, and such detection reagents may be used to detect one or more SNPs of the present invention. One example of a microfluidic system is disclosed in U.S. Pat. No. 5,589,136, which describes the integration of PCR amplification and capillary electrophoresis in chips. Exemplary microfluidic systems comprise a pattern of microchannels designed onto a glass, silicon, quartz, or plastic wafer included on a microchip. The movements of the samples may be controlled by electric, electroosmotic or hydrostatic forces applied across different areas of the microchip to create functional microscopic valves and pumps with no moving parts. Varying the voltage can be used as a means to control the liquid flow at intersections between the micro-machined channels and to change the liquid flow rate for pumping across different sections of the microchip. See, for example, U.S. Pat. No. 6,153,073 (to Dubrow et al.), and U.S. Pat. No. 6,156,181 (to Parce et al).

For genotyping SNPs, a microfluidic system may integrate, for example, nucleic acid amplification, primer extension, capillary electrophoresis, and a detection method such as laser induced fluorescence detection.

Uses of Nucleic Acid Molecules

The nucleic acid molecules of the present invention have a variety of uses, especially in the diagnosis and treatment of endometriosis. For example, the nucleic acid molecules are useful as hybridization probes, such as for genotyping SNPs in messenger RNA, transcript, cDNA, genomic DNA, amplified DNA or other nucleic acid molecules comprising one of the SNPs disclosed in Tables 1-433, as well as their orthologs.

A probe can hybridize to any nucleotide sequence along the entire length of a nucleic acid molecule encompassing a SNP of the present invention. Preferably, a probe of the present invention hybridizes to a region of a target sequence that encompasses a SNP. More preferably, a probe hybridizes to a SNP-containing target sequence in a sequence-specific manner such that it distinguishes the target sequence from other nucleotide sequences which vary from the target sequence only by which nucleotide is present at the SNP site. Such a probe is particularly useful for detecting the presence of a SNP-containing nucleic acid in a test sample, or for determining which nucleotide (allele) is present at a particular SNP site (i.e., genotyping the SNP site).

A nucleic acid hybridization probe may be used for determining the presence, level, form, and/or distribution of nucleic acid expression. The nucleic acid whose level is determined can be DNA or RNA. Accordingly, probes specific for the SNPs described herein can be used to assess the presence, expression and/or gene copy number in a given cell, tissue, or organism. These uses are relevant for diagnosis of disorders involving an increase or decrease in gene expression relative to normal levels. In vitro techniques for detection of mRNA include, for example, Northern blot hybridizations and in situ hybridizations. In vitro techniques for detecting DNA include Southern blot hybridizations and in situ hybridizations (Sambrook J, Russell D W. Molecular Cloning: A Laboratory Manual. Third ed. Woodbury, NY: CSHL Press; 2001).

Probes can be used as part of a diagnostic test kit for identifying cells or tissues in which a variant protein is expressed, such as by measuring the level of a variant protein-encoding nucleic acid (e.g., mRNA) in a sample of cells from a subject or determining if a polynucleotide contains a SNP of interest.

Thus, the nucleic acid molecules of the invention can be used as hybridization probes to detect the SNPs disclosed herein, thereby determining whether an individual with the polymorphisms is at risk for endometriosis or has developed early stage endometriosis. Detection of a SNP associated with an endometriosis phenotype provides a diagnostic and/or a prognostic tool for an active endometriosis and/or genetic predisposition to the endometriosis.

The nucleic acid molecules of the invention are also useful as primers to amplify any given region of a nucleic acid molecule, particularly a region containing a SNP of the present invention.

The nucleic acid molecules of the invention are also useful for constructing vectors containing a gene regulatory region of the nucleic acid molecules of the present invention. Further, the nucleic acid molecules of the invention also have therapeutic use in the form of siRNA (small interfering RNA).

SNP Genotyping Methods

The process of determining which specific nucleotide (i.e., allele) is present at each of one or more SNP positions, such as a SNP position in a nucleic acid molecule characterized by a SNP of the present invention, is referred to as SNP genotyping. The present invention provides methods of SNP genotyping, such as for use in screening for endometriosis or related pathologies, or determining predisposition thereto, or determining responsiveness to a form of treatment, or in genome mapping or SNP association analysis, etc.

Nucleic acid samples can be genotyped to determine which allele(s) is/are present at any given genetic region (e.g., SNP position) of interest by methods well known in the art. The neighboring sequence can be used to design SNP detection reagents such as oligonucleotide probes, which may optionally be implemented in a kit format. Exemplary SNP genotyping methods are described in Chen et al. (Single nucleotide polymorphism genotyping: biochemistry, protocol, cost and throughput. Pharmacogenomics J. 2003; 3(2):77-96); Kwok et al., (Detection of single nucleotide polymorphisms. Curr Issues Mol Biol. April 2003; 5(2):43-60); Shi, (Technologies for individual genotyping: detection of genetic polymorphisms in drug targets and disease genes. Am J Pharmacogenomics. 2002; 2(3):197-205); and Kwok, (Methods for genotyping single nucleotide polymorphisms. Annu Rev Genomics Hum Genet. 2001; 2:235-258). Exemplary techniques for high-throughput SNP genotyping are described in Marnellos, (High-throughput SNP analysis for genetic association studies. Curr Opin Drug Discov Devel. May 2003; 6(3):317-321). Common SNP genotyping methods include, but are not limited to, TaqMan assays, molecular beacon assays, nucleic acid arrays, allele-specific primer extension, allele-specific PCR, arrayed primer extension, homogeneous primer extension assays, primer extension with detection by mass spectrometry, mass spectrometry with or with monoisotopic dNTPs (U.S. Pat. No. 6,734,294), pyrosequencing, multiplex primer extension sorted on genetic arrays, ligation with rolling circle amplification, homogeneous ligation, OLA (U.S. Pat. No. 4,988,167), multiplex ligation reaction sorted on genetic arrays, restriction-fragment length polymorphism, single base extension-tag assays, and the Invader assay. Such methods may be used in combination with detection mechanisms such as, for example, luminescence or chemiluminescence detection, fluorescence detection, time-resolved fluorescence detection, fluorescence resonance energy transfer, fluorescence polarization, mass spectrometry, electrospray mass spectrometry, and electrical detection.

Various methods for detecting polymorphisms include, but are not limited to, methods in which protection from cleavage agents is used to detect mismatched bases in RNA/RNA or RNA/DNA duplexes (Myers R M, et al. A general method for saturation mutagenesis of cloned DNA fragments. Science. Jul. 19, 1985; 229(4710):242-247. Cotton R G H, et al. Reactivity of Cytosine and Thymine in Single-Base-Pair Mismatches with Hydroxylamine and Osmium Tetroxide and Its Application to the Study of Mutations. PNAS. Jun. 15, 1988; 85(12):4397-4401. Saleeba J A, Cotton R G. Chemical cleavage of mismatch to detect mutations. Methods Enzymol. 1993; 217:286-295), comparison of the electrophoretic mobility of variant and wild type nucleic acid molecules (Orita M, et al. Detection of polymorphisms of human DNA by gel electrophoresis as single-strand conformation polymorphisms. Proc Natl Acad Sci USA. April 1989; 86(8):2766-2770. Cotton R G H. Current methods of mutation detection. Mutation Research. 1993; 285:125-144. Hayashi K. PCR-SSCP: a method for detection of mutations. Genet Anal Tech Appl. June 1992; 9(3):73-79), and assaying the movement of polymorphic or wild-type fragments in polyacrylamide gels containing a gradient of denaturant using denaturing gradient gel electrophoresis (DGGE) (Myers R M, et al. Detection of single base substitutions in total genomic DNA. Nature. Feb. 7-13, 1985; 313(6002):495-498). Sequence variations at specific locations can also be assessed by nuclease protection assays such as RNase and S1 protection or chemical cleavage methods.

In a preferred embodiment, SNP genotyping is performed using the TaqMan assay, which is also known as the 5′ nuclease assay (U.S. Pat. Nos. 5,210,015 and 5,538,848). The TaqMan assay detects the accumulation of a specific amplified product during PCR. The TaqMan assay utilizes an oligonucleotide probe labeled with a fluorescent reporter dye and a quencher dye. The reporter dye is excited by irradiation at an appropriate wavelength, it transfers energy to the quencher dye in the same probe via a process called fluorescence resonance energy transfer (FRET). When attached to the probe, the excited reporter dye does not emit a signal. The proximity of the quencher dye to the reporter dye in the intact probe maintains a reduced fluorescence for the reporter. The reporter dye and quencher dye may be at the 5′ most and the 3′ most ends, respectively, or vice versa. Alternatively, the reporter dye may be at the 5′ or 3′ most end while the quencher dye is attached to an internal nucleotide, or vice versa. In yet another embodiment, both the reporter and the quencher may be attached to internal nucleotides at a distance from each other such that fluorescence of the reporter is reduced.

During PCR, the 5′ nuclease activity of DNA polymerase cleaves the probe, thereby separating the reporter dye and the quencher dye and resulting in increased fluorescence of the reporter. Accumulation of PCR product is detected directly by monitoring the increase in fluorescence of the reporter dye. The DNA polymerase cleaves the probe between the reporter dye and the quencher dye only if the probe hybridizes to the target SNP-containing template which is amplified during PCR, and the probe is designed to hybridize to the target SNP site only if a particular SNP allele is present.

Preferred TaqMan primer and probe sequences can readily be determined using the SNP and associated nucleic acid sequence information provided herein. A number of computer programs, such as Primer Express (Applied Biosystems, Foster City, Calif.), can be used to rapidly obtain optimal primer/probe sets. It will be apparent to one of skill in the art that such primers and probes for detecting the SNPs of the present invention are useful in diagnostic assays for endometriosis and related pathologies, and can be readily incorporated into a kit format. The present invention also includes modifications of the Taqman assay well known in the art such as the use of Molecular Beacon probes (U.S. Pat. Nos. 5,118,801 and 5,312,728) and other variant formats (U.S. Pat. Nos. 5,866,336 and 6,117,635).

Another preferred method for genotyping the SNPs of the present invention is the use of two oligonucleotide probes in an OLA (see, e.g., U.S. Pat. No. 4,988,617). In this method, one probe hybridizes to a segment of a target nucleic acid with its 3′ most end aligned with the SNP site. A second probe hybridizes to an adjacent segment of the target nucleic acid molecule directly 3′ to the first probe. The two juxtaposed probes hybridize to the target nucleic acid molecule, and are ligated in the presence of a linking agent such as a ligase if there is perfect complementarity between the 3′ most nucleotide of the first probe with the SNP site. If there is a mismatch, ligation would not occur. After the reaction, the ligated probes are separated from the target nucleic acid molecule, and detected as indicators of the presence of a SNP.

The following patents, patent applications, and published international patent applications, which are all hereby incorporated by reference, provide additional information pertaining to techniques for carrying out various types of OLA: U.S. Pat. Nos. 6,027,889, 6,268,148, 5,494,810, 5,830,711, and 6,054,564 describe OLA strategies for performing SNP detection; WIPO patents WO/1997/031256 and WO/2000/056927 describe OLA strategies for performing SNP detection using universal arrays, wherein a zipcode sequence can be introduced into one of the hybridization probes, and the resulting product, or amplified product, hybridized to a universal zip code array; WIPO patent WO/2001/092579 (and U.S. application Ser. No. 09/584,905) describes OLA (or LDR) followed by PCR, wherein zipcodes are incorporated into OLA probes, and amplified PCR products are determined by electrophoretic or universal zipcode array readout; U.S. application 60/427,818, 60/445,636, and 60/445,494 describe SNPlex methods and software for multiplexed SNP detection using OLA followed by PCR, wherein zipcodes are incorporated into OLA probes, and amplified PCR products are hybridized with a zipchute reagent, and the identity of the SNP determined from electrophoretic readout of the zipchute. In some embodiments, OLA is carried out prior to PCR (or another method of nucleic acid amplification). In other embodiments, PCR (or another method of nucleic acid amplification) is carried out prior to OLA.

Another method for SNP genotyping is based on mass spectrometry. Mass spectrometry takes advantage of the unique mass of each of the four nucleotides of DNA. SNPs can be unambiguously genotyped by mass spectrometry by measuring the differences in the mass of nucleic acids having alternative SNP alleles. MALDI-TOF (Matrix Assisted Laser Desorption Ionization-Time of Flight) mass spectrometry technology is preferred for extremely precise determinations of molecular mass, such as SNPs. Numerous approaches to SNP analysis have been developed based on mass spectrometry. Preferred mass spectrometry-based methods of SNP genotyping include primer extension assays, which can also be utilized in combination with other approaches, such as traditional gel-based formats and microarrays.

The following references provide further information describing mass spectrometry-based methods for SNP genotyping: Bocker (SNP and mutation discovery using base-specific cleavage and MALDI-TOF mass spectrometry. Bioinformatics. 2003; 19 Suppl 1:i44-53), Storm et al. (MALDI-TOF mass spectrometry-based SNP genotyping. Methods Mol Biol. 2003; 212:241-262), Jurinke et al. (The use of MassARRAY technology for high throughput genotyping. Adv Biochem Eng Biotechnol. 2002; 77:57-74), and Jurinke et al. (Automated genotyping using the DNA MassArray technology. Methods Mol Biol. 2002; 187:179-192).

An even more preferred method for genotyping the SNPs of the present invention is the use of electrospray mass spectrometry for direct analysis of an amplified nucleic acid (see, e.g., U.S. Pat. No. 6,734,294). In this method, in one aspect, an amplified nucleic acid product may be isotopically enriched in an isotope of oxygen (O), carbon (C), nitrogen (N) or any combination of those elements. In a preferred embodiment the amplified nucleic acid is isotopically enriched to a level of greater than 99.9% in the elements of O16, C12 and N14. The amplified isotopically enriched product can then be analyzed by electrospray mass spectrometry to determine the nucleic acid composition and the corresponding SNP genotyping. Isotopically enriched amplified products result in a corresponding increase in sensitivity and accuracy in the mass spectrum. In another aspect of this method an amplified nucleic acid that is not isotopically enriched can also have composition and SNP genotype determined by electrospray mass spectrometry.

SNPs can also be scored by direct DNA sequencing. A variety of automated sequencing procedures can be utilized (Naeve C W, et al. Accuracy of automated DNA sequencing: a multi-laboratory comparison of sequencing results. Biotechniques. September 1995; 19(3):448-453), including sequencing by mass spectrometry (see, e.g., WIPO patent WO/1994/016101; Cohen A S, et al. Emerging technologies for sequencing antisense oligonucleotides: capillary electrophoresis and mass spectrometry. Adv Chromatogr. 1996; 36:127-162; and Griffin H G, Griffin A M. DNA sequencing. Recent innovations and future trends. Appl Biochem Biotechnol. January-February 1993; 38(1-2):147-159). The nucleic acid sequences of the present invention enable one of ordinary skill in the art to readily design sequencing primers for such automated sequencing procedures. Commercial instrumentation, such as the Applied Biosystems 377, 3100, 3700, 3730, and 3730×1 DNA Analyzers (Foster City, Calif.), is commonly used in the art for automated sequencing.

SNP genotyping can include the steps of, for example, collecting a biological sample from a human subject (e.g., sample of tissues, cells, fluids, secretions, etc.), isolating nucleic acids (e.g., genomic DNA, mRNA or both) from the cells of the sample, contacting the nucleic acids with one or more primers which specifically hybridize to a region of the isolated nucleic acid containing a target SNP under conditions such that hybridization and amplification of the target nucleic acid region occurs, and determining the nucleotide present at the SNP position of interest, or, in some assays, detecting the presence or absence of an amplification product (assays can be designed so that hybridization and/or amplification will only occur if a particular SNP allele is present or absent). In some assays, the size of the amplification product is detected and compared to the length of a control sample; for example, deletions and insertions can be detected by a change in size of the amplified product compared to a normal genotype.

SNP genotyping is useful for numerous practical applications, as described below. Examples of such applications include, but are not limited to, SNP-endometriosis association analysis, endometriosis predisposition screening, endometriosis diagnosis, endometriosis prognosis, endometriosis progression monitoring, determining therapeutic strategies based on an individual's genotype, and stratifying a patient population for clinical trials for a treatment such as minimally invasive device for the treatment of endometriosis.

Analysis of Genetic Association Between SNPs and Phenotypic Traits

SNP genotyping for endometriosis diagnosis, endometriosis predisposition screening, endometriosis prognosis and endometriosis treatment and other uses described herein, typically relies on initially establishing a genetic association between one or more specific SNPs and the particular phenotypic traits of interest.

In a genetic association study, the cause of interest to be tested is a certain allele or a SNP or a combination of alleles or a haplotype from several SNPs. Thus, tissue specimens (e.g., saliva) from the sampled individuals may be collected and genomic DNA genotyped for the SNP(s) of interest. In addition to the phenotypic trait of interest, other information such as demographic (e.g., age, gender, ethnicity, etc.), clinical, and environmental information that may influence the outcome of the trait can be collected to further characterize and define the sample set. Specifically, in an endometriosis genetic association study, clinical information such as body mass index, age and diet may be collected. In many cases, these factors are known to be associated with diseases and/or SNP allele frequencies. There are likely gene-environment and/or gene-gene interactions as well. Analysis methods to address gene-environment and gene-gene interactions (for example, the effects of the presence of both susceptibility alleles at two different genes can be greater than the effects of the individual alleles at two genes combined) are discussed below.

After all the relevant phenotypic and genotypic information has been obtained, statistical analyses are carried out to determine if there is any significant correlation between the presence of an allele or a genotype with the phenotypic characteristics of an individual. Preferably, data inspection and cleaning are first performed before carrying out statistical tests for genetic association. Epidemiological and clinical data of the samples can be summarized by descriptive statistics with tables and graphs. Data validation is preferably performed to check for data completion, inconsistent entries, and outliers. Chi-squared tests may then be used to check for significant differences between cases and controls for discrete and continuous variables, respectively. To ensure genotyping quality, Hardy-Weinberg disequilibrium tests can be performed on cases and controls separately. Significant deviation from Hardy-Weinberg equilibrium (HWE) in both cases and controls for individual markers can be indicative of genotyping errors. If HWE is violated in a majority of markers, it is indicative of population substructure that should be further investigated. Moreover, Hardy-Weinberg disequilibrium in cases only can indicate genetic association of the markers with the disease of interest. (Weir B S. Genetic Data Analysis: Methods for Discrete Population Genetic Data. Sunderland, Mass.: Sinauer Associates; 1990).

To test whether an allele of a single SNP is associated with the case or control status of a phenotypic trait, one skilled in the art can compare allele frequencies in cases and controls. Standard chi-squared tests and Fisher exact tests can be carried out on a 2×2 table (2 SNP alleles×2 outcomes in the categorical trait of interest). To test whether genotypes of a SNP are associated, chi-squared tests can be carried out on a 3×2 table (3 genotypes×2 outcomes). Score tests are also carried out for genotypic association to contrast the three genotypic frequencies (major homozygotes, heterozygotes and minor homozygotes) in cases and controls, and to look for trends using 3 different modes of inheritance, namely dominant (with contrast coefficients 2, −1, −1), additive (with contrast coefficients 1, 0, −1) and recessive (with contrast coefficients 1, 1, −2). Odds ratios for minor versus major alleles, and odds ratios for heterozygote and homozygote variants versus the wild type genotypes are calculated with the desired confidence limits, usually 95%. In the present study a software algorithm, PLINK, has been applied to automate the calculation of Hardy-Weinberg equilibrium, chi-square, p-values and odds-ratios for very large numbers of SNPs and Case-Control individuals simultaneously (Purcell S, et al. PLINK: a tool set for whole-genome association and population-based linkage analyses. Am J Hum Genet. September 2007; 81(3):559-575).

In order to control for confounding effects and to test for interactions a stepwise multiple logistic regression analysis using statistical packages such as SAS or R may be performed. Logistic regression is a model-building technique in which the best fitting and most parsimonious model is built to describe the relation between the dichotomous outcome (for instance, getting a certain endometriosis or not) and a set of independent variables (for instance, genotypes of different associated genes, and the associated demographic and environmental factors). The most common model is one in which the logit transformation of the odds ratios is expressed as a linear combination of the variables (main effects) and their cross-product terms (interactions) (Hosmer D W, Lemeshow S. Applied Logistic Regression. Second ed. Hoboken, N.J.: Wiley-Interscience; 2000). To test whether a certain variable or interaction is significantly associated with the outcome, coefficients in the model are first estimated and then tested for statistical significance of their departure from zero.

In addition to performing association tests one marker at a time, haplotype association analysis may also be performed to study a number of markers that are closely linked together. Haplotype association tests can have better power than genotypic or allelic association tests when the tested markers are not the disease-causing mutations themselves but are in linkage disequilibrium with such mutations. In order to perform haplotype association effectively, marker-marker linkage disequilibrium measures, both D′ and r², are typically calculated for the markers within a gene to elucidate the haplotype structure. Recent studies (Daly M J, et al. High-resolution haplotype structure in the human genome. Nat Genet. October 2001; 29(2):229-232) in linkage disequilibrium indicate that SNPs within a gene are organized in block pattern, and a high degree of linkage disequilibrium exists within blocks and very little linkage disequilibrium exists between blocks. Haplotype association with the endometriosis status can be performed using such blocks once they have been elucidated.

Haplotype association tests can be carried out in a similar fashion as the allelic and genotypic association tests. Each haplotype is analogous to an allele in a multi-allelic marker. One skilled in the art can either compare the haplotype frequencies in cases and controls or test genetic association with different pairs of haplotypes. It has been proposed (Schaid D J, et al. Score tests for association between traits and haplotypes when linkage phase is ambiguous. Am J Hum Genet. February 2002; 70(2):425-434) that score tests can be done on haplotypes using the program “haplo.score”. In that method, haplotypes are first inferred by EM algorithm and score tests are carried out with a generalized linear model (GLM) framework that allows the adjustment of other factors.

An important decision in the performance of genetic association tests is the determination of the significance level at which significant association can be declared when the p-value of the tests reaches that level. In an exploratory analysis where positive hits will be followed up in subsequent confirmatory testing, an unadjusted p-value <0.1 (a significance level on the lenient side) may be used for generating hypotheses for significant association of a SNP with certain phenotypic characteristics of endometriosis. It is preferred that a p-value <0.05 (a significance level traditionally used in the art) is achieved in order for a SNP to be considered to have an association with endometriosis. It is more preferred that a p-value <0.01 (a significance level on the stringent side) is achieved for an association to be declared. Permutation tests to control for the false discovery rates, FDR, can further be employed (Schaid D J, et al. Score tests for association between traits and haplotypes when linkage phase is ambiguous. Am J Hum Genet. February 2002; 70(2):425-434). Such methods to control for multiplicity would be preferred when the tests are dependent and controlling for false discovery rates is sufficient as opposed to controlling for the experiment-wise error rates.

In replication studies using samples from different populations after statistically significant markers have been identified in the exploratory stage, meta-analyses can then be performed by combining evidence of different studies, such as the original study and the replication study (Rothman K J, Greenland S, eds. Modern Epidemiology. Second ed. Philadelphia, Pa.: Lippincott Williams & Wilkins; 1998, 643-673). If available, association results known in the art for the same SNPs can be included in the meta-analyses.

Since both genotyping and endometriosis status classification can involve errors, sensitivity analyses may be performed to see how odds ratios and p-values would change upon various estimates on genotyping and endometriosis classification error rates.

Once individual risk factors, genetic or non-genetic, have been found for the predisposition to endometriosis, the next step is to set up a classification/prediction scheme to predict the category (for instance, endometriosis or no endometriosis) that an individual will be in depending on his genotypes of associated SNPs and other non-genetic risk factors. Logistic regression for discrete trait and linear regression for continuous trait are standard techniques for such tasks (Draper N R, Smith H. Applied Regression Analysis. Third ed. Hoboken, N.J.: Wiley-Interscience; 1998). Moreover, other techniques can also be used for setting up classification. Such techniques include, but are not limited to, MART, CART, neural network, and discriminant analyses that are suitable for use in comparing the performance of different methods (Hastie T, et al. The Elements of Statistical Learning: Data Mining, Inference, and Prediction. New York: Springer; 2001).

Endometriosis Diagnosis and Predisposition Screening

Information on association/correlation between genotypes and endometriosis-related phenotypes can be exploited in several ways. For example, in the case of a highly statistically significant association between one or more SNPs with predisposition to a disease for which treatment is available, detection of such a genotype pattern in an individual may justify particular treatment, or at least the institution of regular monitoring of the individual. In the case of a weaker but still statistically significant association between a SNP and a human disease, immediate therapeutic intervention or monitoring may not be justified after detecting the susceptibility allele or SNP.

The SNPs of the invention may contribute to endometriosis in an individual in different ways. Some polymorphisms occur within a protein coding sequence and contribute to endometriosis phenotype by affecting protein structure. Other polymorphisms occur in noncoding regions but may exert phenotypic effects indirectly via influence on, for example, replication, transcription, and/or translation. A single SNP may affect more than one phenotypic trait. Likewise, a single phenotypic trait may be affected by multiple SNPs in different genes.

Haplotypes are particularly useful in that, for example, fewer SNPs can be genotyped to determine if a particular genomic region harbors a locus that influences a particular phenotype, such as in linkage disequilibrium-based SNP association analysis.

Linkage disequilibrium (LD) refers to the co-inheritance of alleles (e.g., alternative nucleotides) at two or more different SNP sites at frequencies greater than would be expected from the separate frequencies of occurrence of each allele in a given population. The expected frequency of co-occurrence of two alleles that are inherited independently is the frequency of the first allele multiplied by the frequency of the second allele. Alleles that co-occur at expected frequencies are said to be in “linkage equilibrium”. In contrast, LD refers to any non-random genetic association between allele(s) at two or more different SNP sites, which is generally due to the physical proximity of the two loci along a chromosome. LD can occur when two or more SNPs sites are in close physical proximity to each other on a given chromosome and therefore alleles at these SNP sites will tend to remain unseparated for multiple generations with the consequence that a particular nucleotide (allele) at one SNP site will show a non-random association with a particular nucleotide (allele) at a different SNP site located nearby. Hence, genotyping one of the SNP sites will give almost the same information as genotyping the other SNP site that is in LD.

For diagnostic purposes, if a particular SNP site is found to be useful for diagnosing endometriosis, then the skilled artisan would recognize that other SNP sites which are in LD with this SNP site would also be useful for diagnosing the condition. Various degrees of LD can be encountered between two or more SNPs with the result being that some SNPs are more closely associated (i.e., in stronger LD) than others. Furthermore, the physical distance over which LD extends along a chromosome differs between different regions of the genome, and therefore the degree of physical separation between two or more SNP sites necessary for LD to occur can differ between different regions of the genome.

For diagnostic applications, polymorphisms (e.g., SNPs and/or haplotypes) that are not the actual disease-causing (causative) polymorphisms, but are in LD with such causative polymorphisms, are also useful. In such instances, the genotype of the polymorphism(s) that is/are in LD with the causative polymorphism is predictive of the genotype of the causative polymorphism and, consequently, predictive of the phenotype (e.g., endometriosis) that is influenced by the causative SNP(s). Thus, polymorphic markers that are in LD with causative polymorphisms are useful as diagnostic markers, and are particularly useful when the actual causative polymorphism(s) is/are unknown.

Linkage disequilibrium in the human genome is reviewed in: International HapMap Consortium, (A haplotype map of the human genome. Nature. Oct. 27, 2005; 437(7063):1299-1320); Wall and Pritchard (Haplotype blocks and linkage disequilibrium in the human genome. Nat Rev Genet. August 2003; 4(8):587-597); Garner and Slatkin (On selecting markers for association studies: patterns of linkage disequilibrium between two and three diallelic loci. Genet Epidemiol. January 2003; 24(1):57-67); Ardlie et al. (Patterns of linkage disequilibrium in the human genome. Nat Rev Genet. April 2002; 3(4):299-309); and Remm and Metspalu (High-density genotyping and linkage disequilibrium in the human genome using chromosome 22 as a model. Curr Opin Chem Biol. February 2002; 6(1):24-30).

The contribution or association of particular SNPs and/or SNP haplotypes with endometriosis phenotypes, such as endometriosis, enables the SNPs of the present invention to be used to develop superior diagnostic tests capable of identifying individuals who express a detectable trait, such as endometriosis as the result of a specific genotype, or individuals whose genotype places them at an increased or decreased risk of developing a detectable trait at a subsequent time as compared to individuals who do not have that genotype. As described herein, diagnostics may be based on a single SNP or a group of SNPs. Combined detection of a plurality of SNPs (for example, 2, 3, 4, 5, 6, 7, 8, 9, 10, 11, 12, 13, 14, 15, 16, 17, 18, 19, 20, 24, 25, 30, 32, 48, 50, 64, 96, 100, or any other number in-between, or more, of the SNPs provided in Tables 1-433 typically increases the probability of an accurate diagnosis. For example, the presence of a single SNP known to correlate with endometriosis might indicate a odds ratio of 1.5 that an individual has or is at risk of developing endometriosis, whereas detection of five SNPs, each of which correlates with endometriosis, might indicate an odds ratio of 9.5 that an individual has or is at risk of developing endometriosis. To further increase the accuracy of diagnosis or predisposition screening, analysis of the SNPs of the present invention can be combined with that of other polymorphisms or other risk factors of endometriosis, such as pelvic pain and infertility.

It will, of course, be understood by practitioners skilled in the treatment or diagnosis of endometriosis that the present invention generally does not intend to provide an absolute identification of individuals who are at risk (or less at risk) of developing endometriosis and/or pathologies related to endometriosis, but rather to indicate a certain increased (or decreased) degree or likelihood of developing the endometriosis based on statistically significant association results. However, this information is extremely valuable as it can be used to, for example, initiate earlier preventive treatments or to allow an individual carrying one or more significant SNPs or SNP haplotypes to regularly scheduled physical exams to monitor for the appearance or change of their endometriosis in order to identify and begin treatment of the endometriosis at an early stage.

The diagnostic techniques of the present invention may employ a variety of methodologies to determine whether a test subject has a SNP or a SNP pattern associated with an increased or decreased risk of developing a detectable trait or whether the individual suffers from a detectable trait as a result of a particular polymorphism/mutation, including, for example, methods which enable the analysis of individual chromosomes for haplotyping, family studies, single sperm DNA analysis, or somatic hybrids. The trait analyzed using the diagnostics of the invention may be any detectable trait that is commonly observed in pathologies and disorders related to endometriosis.

Another aspect of the present invention relates to a method of determining whether an individual is at risk (or less at risk) of developing one or more traits or whether an individual expresses one or more traits as a consequence of possessing a particular trait-causing or trait-influencing allele. These methods generally involve obtaining a nucleic acid sample from an individual and assaying the nucleic acid sample to determine which nucleotide(s) is/are present at one or more SNP positions, wherein the assayed nucleotide(s) is/are indicative of an increased or decreased risk of developing the trait or indicative that the individual expresses the trait as a result of possessing a particular trait-causing or trait-influencing allele.

The SNPs of the present invention also can be used to identify novel therapeutic targets for endometriosis. For example, genes containing the disease-associated variants (“variant genes”) or their products, as well as genes or their products that are directly or indirectly regulated by or interacting with these variant genes or their products, can be targeted for the development of therapeutics that, for example, treat the endometriosis or prevent or delay endometriosis onset. The therapeutics may be composed of, for example, small molecules, proteins, protein fragments or peptides, antibodies, nucleic acids, or their derivatives or mimetics which modulate the functions or levels of the target genes or gene products.

The SNPs/haplotypes of the present invention are also useful for improving many different aspects of the drug development process. For example, individuals can be selected for clinical trials based on their SNP genotype. Individuals with SNP genotypes that indicate that they are most likely to respond to or most likely to benefit from a device or a drug can be included in the trials and those individuals whose SNP genotypes indicate that they are less likely to or would not respond to a device or a drug, or suffer adverse reactions, can be eliminated from the clinical trials. This not only improves the safety of clinical trials, but also will enhance the chances that the trial will demonstrate statistically significant efficacy. Furthermore, the SNPs of the present invention may explain why certain previously developed devices or drugs performed poorly in clinical trials and may help identify a subset of the population that would benefit from a drug that had previously performed poorly in clinical trials, thereby “rescuing” previously developed therapeutic treatment methods or drugs, and enabling the methods or drug to be made available to a particular endometriosis patient population that can benefit from it.

Pharmaceutical Compositions

Any of the endometriosis-associated proteins, and encoding nucleic acid molecules, disclosed herein can be used as therapeutic targets (or directly used themselves as therapeutic compounds) for treating endometriosis and related pathologies, and the present disclosure enables therapeutic compounds (e.g., small molecules, antibodies, therapeutic proteins, RNAi and antisense molecules, etc.) to be developed that target (or are comprised of) any of these therapeutic targets.

Variant Proteins Encoded by SNP-Containing Nucleic Acid Molecules

The present invention provides SNP-containing nucleic acid molecules, some of which encode proteins having variant amino acid sequences as compared to the art-known (i.e., wild-type) proteins. These variants will generally be referred to herein as variant proteins/peptides/polypeptides, or polymorphic proteins/peptides/polypeptides of the present invention. The terms “protein”, “peptide”, and “polypeptide” are used herein interchangeably.

A variant protein of the present invention may be encoded by, for example, a nonsynonymous nucleotide substitution at any one of the cSNP positions disclosed herein. In addition, variant proteins may also include proteins whose expression, structure, and/or function is altered by a SNP disclosed herein, such as a SNP that creates or destroys a stop codon, a SNP that affects splicing, and a SNP in control/regulatory elements, e.g. promoters, enhancers, or transcription factor binding domains.

Uses of Variant Proteins

The variant proteins of the present invention can be used in a variety of ways, including but not limited to, in assays to determine the biological activity of a variant protein, such as in a panel of multiple proteins for high-throughput screening; to raise antibodies or to elicit another type of immune response; as a reagent (including the labeled reagent) in assays designed to quantitatively determine levels of the variant protein (or its binding partner) in biological fluids; as a marker for cells or tissues in which it is preferentially expressed (either constitutively or at a particular stage of tissue differentiation or development or in a endometriosis state); as a target for screening for a therapeutic agent; and as a direct therapeutic agent to be administered into a human subject. Any of the variant proteins disclosed herein may be developed into reagent grade or kit format for commercialization as research products. Methods for performing the uses listed above are well known to those skilled in the art (see, e.g., Sambrook J, Russell D W. Molecular Cloning: A Laboratory Manual. Third ed. Woodbury, NY: CSHL Press; 2001 and Berger S L, Kimmel A R, eds. Guide to Molecular Cloning Techniques. New York: Academic Press; 1987. Methods in Enzymology; No. 152).

Computer-Related Embodiments

The SNPs provided in the present invention may be “provided” in a variety of mediums to facilitate use thereof. As used in this section, “provided” refers to a manufacture, other than an isolated nucleic acid molecule, that contains SNP information of the present invention. Such a manufacture provides the SNP information in a form that allows a skilled artisan to examine the manufacture using means not directly applicable to examining the SNPs or a subset thereof as they exist in nature or in purified form. The SNP information that may be provided in such a form includes any of the SNP information provided by the present invention such as, for example, polymorphic nucleic acid and/or amino acid sequence information, information about observed SNP alleles, alternative codons, populations, allele frequencies, SNP types, and/or affected proteins, or any other information provided by the present invention in Table 1 or in Tables 3-433.

In one application of this embodiment, the SNPs of the present invention can be recorded on a computer readable medium. As used herein, “computer readable medium” refers to any medium that can be read and accessed directly by a computer. Such media include, but are not limited to: magnetic storage media, such as floppy discs, hard disc storage medium, and magnetic tape; optical storage media such as CD-ROM; electrical storage media such as RAM and ROM; and hybrids of these categories such as magnetic/optical storage media. A skilled artisan can readily appreciate how any of the presently known computer readable media can be used to create a manufacture comprising computer readable medium having recorded thereon a nucleotide sequence of the present invention. One such medium is provided with the present application, namely, the present application contains computer readable medium (CD-R) that has nucleic acid sequences (and encoded protein sequences) containing SNPs provided/recorded thereon in ASCII text format in a Sequence Listing along with accompanying Tables that contain detailed SNP and sequence information.

As used herein, “recorded” refers to a process for storing information on computer readable medium. A skilled artisan can readily adopt any of the presently known methods for recording information on computer readable medium to generate manufactures comprising the SNP information of the present invention.

A variety of data storage structures are available to a skilled artisan for creating a computer readable medium having recorded thereon a nucleotide or amino acid sequence of the present invention. The choice of the data storage structure will generally be based on the means chosen to access the stored information. In addition, a variety of data processor programs and formats can be used to store the nucleotide/amino acid sequence information of the present invention on computer readable medium. For example, the sequence information can be represented in a word processing text file, formatted in commercially-available software such as WordPerfect and Microsoft Word, represented in the form of an ASCII file, or stored in a database application, such as OB2, Sybase, Oracle, or the like. A skilled artisan can readily adapt any number of data processor structuring formats (e.g., text file or database) in order to obtain computer readable medium having recorded thereon the SNP information of the present invention.

By providing the SNPs of the present invention in computer readable form, a skilled artisan can routinely access the SNP information for a variety of purposes. Computer software is publicly available which allows a skilled artisan to access sequence information provided in a computer readable medium. Examples of publicly available computer software include BLAST (Altschul S F, et al. Basic local alignment search tool. J Mol Biol. Oct. 5, 1990; 215(3):403-410) and BLAZE (Brutlag D L, et al. BLAZE: An implementation of the Smith-Waterman comparison algorithm on a massively parallel computer. Computers and Chemistry. 1993; 17:203-207) search algorithms.

The present invention further provides systems, particularly computer-based systems, which contain the SNP information described herein. Such systems may be designed to store and/or analyze information on, for example, a large number of SNP positions, or information on SNP genotypes from a large number of individuals. The SNP information of the present invention represents a valuable information source. The SNP information of the present invention stored/analyzed in a computer-based system may be used for such computer-intensive applications as determining or analyzing SNP allele frequencies in a population, mapping endometriosis genes, genotype-phenotype association studies, grouping SNPs into haplotypes, correlating SNP haplotypes with response to particular treatments or for various other bioinformatic, pharmacogenomic or drug development.

As used herein, “a computer-based system” refers to the hardware means, software means, and data storage means used to analyze the SNP information of the present invention. The minimum hardware means of the computer-based systems of the present invention typically comprises a central processing unit (CPU), input means, output means, and data storage means. A skilled artisan can readily appreciate that any one of the currently available computer-based systems are suitable for use in the present invention. Such a system can be changed into a system of the present invention by utilizing the SNP information provided on the CD-R, or a subset thereof, without any experimentation.

As stated above, the computer-based systems of the present invention comprise a data storage means having stored therein SNPs of the present invention and the necessary hardware means and software means for supporting and implementing a search means. As used herein, “data storage means” refers to memory which can store SNP information of the present invention, or a memory access means which can access manufactures having recorded thereon the SNP information of the present invention.

As used herein, “search means” refers to one or more programs or algorithms that are implemented on the computer-based system to identify or analyze SNPs in a target sequence based on the SNP information stored within the data storage means. Search means can be used to determine which nucleotide is present at a particular SNP position in the target sequence. As used herein, a “target sequence” can be any DNA sequence containing the SNP position(s) to be searched or queried.

As used herein, “a target structural motif,” or “target motif,” refers to any rationally selected sequence or combination of sequences containing a SNP position in which the sequence(s) is chosen based on a three-dimensional configuration that is formed upon the folding of the target motif. There are a variety of target motifs known in the art. Protein target motifs include, but are not limited to, enzymatic active sites and signal sequences. Nucleic acid target motifs include, but are not limited to, promoter sequences, hairpin structures, and inducible expression elements (protein binding sequences).

A variety of structural formats for the input and output means can be used to input and output the information in the computer-based systems of the present invention. An exemplary format for an output means is a display that depicts the presence or absence of specified nucleotides (alleles) at particular SNP positions of interest. Such presentation can provide a rapid, binary scoring system for many SNPs simultaneously.

### Examples

### Overview of Association Study

Endometriosis is a debilitating disease, characterized by the presence of endometrium (glands and stroma) at sites outside of the uterus, which is estimated to affect approximately 14% of all women. Endometrioses often leads to pain, local inflammation, scarring and decreased fertility. This example identifies genetic loci in the form of SNPs associated with endometriosis.

A Genome Wide Association study was performed to identify SNPs associated with Endometriosis. The Affymetrix 6.0 GeneChip technology platform was employed in the study to ascertain genotypic information across a total of 906,600 individual SNPs. In all, 1490 individuals diagnosed with Endometriosis were tested and compared to 1469 control individuals. All individuals were of Caucasian descent as determined by Principal Component Analysis implemented in the computer algorithm EIGENSTRAT (Price A L et al. “Principal components analysis corrects for stratification in genome-wide association studies.” Nat Genet. 38(8):904-9, 2006.

A statistical software tool, PLINK, specifically developed to test for genetic association, was used to calculate p values for each SNP, enabling identification of a set of candidate SNPs that showed statistically significant association to Endometriosis.

Scanning the Entire Genome

The Affymetrix GeneChip 6.0 mapping array was used to scan the whole genome. Briefly, 250 ng of genomic DNA was digested with either NspI or Styl restriction endonuclease and digested fragments were ligated to adapters that contained a universal sequence. The ligated products were then amplified using the polymerase chain reaction (PCR) to amplify fragments between 250-2000 bp in length. The PCR products were purified and diluted to a standard concentration. Furthermore, the PCR products were then fragmented with a DNase enzyme to approximately 25-150 bp in length. This fragmentation process further reduced the complexity of the genomic sample. The fragmented PCR products were then labeled with a biotin/streptavidin system and allowed to hybridize to the microarray. After hybridization the arrays were stained and non-specific binding was removed through a series of increasingly stringent washes. The genotypes were determined by fluorescent signal detection in an Affymetrix GCS 3000 scanner. Finally, genotypes were called using the BIRDSEED algorithm which is integrated into Affymetrix PowerTool software.

Selection of SNPs for Quality and Association

A SNP is a DNA sequence variation, occurring when a single nucleotide—adenine (A), thymine (T), cytosine (C) or guanine (G)—in the genome differs between individuals. A variation must occur in at least 1% of the population to be considered a SNP. Variations that occur in less than 1% of the population are, by definition considered to be mutations whether they cause disease or not. SNPs make up 90% of all human genetic variations, and occur every 300 to 1000 bases along the human genome. On average, two of every three SNPs substitute cytosine (C) with thymine (T). For the data to be considered valid for an individual chip, an Affymetrix Contrast Quality Control (CQC) value ≧0.4 was required. SNPs with rare minor allele frequencies, having less than 3% apparent variation overall, were also eliminated from analysis. In addition, SNPs that failed a Hardy-Weinberg equilibrium test in the control population only, using a p-value threshold of 0.001, were also eliminated. After filtering, about 580,000 SNPs were available for analysis. Genotypes were analyzed for significance using PLINK and LD blocks were identified using Haploview 4.2 software using the HapMap dataset release 27 as a reference.

GeneChip microarrays consist of small DNA fragments (referred to as probes), chemically synthesized at specific locations on a coated quartz surface. The precise location where each probe is synthesized is called a feature, and millions of features can be contained on one array. The probes which represent a sequence known to contain a human SNP were selected by Affymetrix based on reliability, sensitivity and specificity. In addition to these criteria, the probes were selected to cover the human genome at approximately equal intervals.

Identification of Endometriosis Affected Individuals.

Individuals were determined to have endometriosis after medical record review by a single physician. In this study, only patients with visually confirmed disease (either by laparoscopy or other surgical intervention) were included as cases. The controls included individuals who represented population controls of matched ethnicity.

Endometriosis Associated SNPs

After sorting all remaining candidate SNPs by p-value, 656 non-redundant SNPs with p-values less than 0.001 were selected as Primary SNPs and are listed in Table 1.

Linkage Disequilibrium Blocks

As described above, the human genome includes extensive regions of linkage disequilibrium that undergo minimal recombination. As a result, any SNP located within the same LD blocks as any of the primary SNPs listed in Table 1 contributes haplotype information for refined diagnostic discrimination and to the further identification of the causative mutation. Therefore, by virtue of linkage disequilibrium, a set of additional

SNPs that have been determined to be in linkage disequilibrium with any of the primary SNPs are listed in Tables 3-433. Specifically, by using the Haploview software package in conjunction with the Caucasian population of the HapMap data set (release 27) LD blocks were identified around the SNPs listed in Table 1. Each of the Tables 3-433 represent SNPs located within the LD block(s) surrounding SNPs from Table 1.

Tables

Lengthy table referenced here
US20150361494A1-20151217-T00001
Please refer to the end of the specification for access instructions.

Lengthy table referenced here
US20150361494A1-20151217-T00002
Please refer to the end of the specification for access instructions.

Lengthy table referenced here
US20150361494A1-20151217-T00003
Please refer to the end of the specification for access instructions.

Lengthy table referenced here
US20150361494A1-20151217-T00004
Please refer to the end of the specification for access instructions.

Lengthy table referenced here
US20150361494A1-20151217-T00005
Please refer to the end of the specification for access instructions.

Lengthy table referenced here
US20150361494A1-20151217-T00006
Please refer to the end of the specification for access instructions.

Lengthy table referenced here
US20150361494A1-20151217-T00007
Please refer to the end of the specification for access instructions.

Lengthy table referenced here
US20150361494A1-20151217-T00008
Please refer to the end of the specification for access instructions.

Lengthy table referenced here
US20150361494A1-20151217-T00009
Please refer to the end of the specification for access instructions.

Lengthy table referenced here
US20150361494A1-20151217-T00010
Please refer to the end of the specification for access instructions.

Lengthy table referenced here
US20150361494A1-20151217-T00011
Please refer to the end of the specification for access instructions.

Lengthy table referenced here
US20150361494A1-20151217-T00012
Please refer to the end of the specification for access instructions.

Lengthy table referenced here
US20150361494A1-20151217-T00013
Please refer to the end of the specification for access instructions.

Lengthy table referenced here
US20150361494A1-20151217-T00014
Please refer to the end of the specification for access instructions.

Lengthy table referenced here
US20150361494A1-20151217-T00015
Please refer to the end of the specification for access instructions.

Lengthy table referenced here
US20150361494A1-20151217-T00016
Please refer to the end of the specification for access instructions.

Lengthy table referenced here
US20150361494A1-20151217-T00017
Please refer to the end of the specification for access instructions.

Lengthy table referenced here
US20150361494A1-20151217-T00018
Please refer to the end of the specification for access instructions.

Lengthy table referenced here
US20150361494A1-20151217-T00019
Please refer to the end of the specification for access instructions.

Lengthy table referenced here
US20150361494A1-20151217-T00020
Please refer to the end of the specification for access instructions.

Lengthy table referenced here
US20150361494A1-20151217-T00021
Please refer to the end of the specification for access instructions.

Lengthy table referenced here
US20150361494A1-20151217-T00022
Please refer to the end of the specification for access instructions.

Lengthy table referenced here
US20150361494A1-20151217-T00023
Please refer to the end of the specification for access instructions.

Lengthy table referenced here
US20150361494A1-20151217-T00024
Please refer to the end of the specification for access instructions.

Lengthy table referenced here
US20150361494A1-20151217-T00025
Please refer to the end of the specification for access instructions.

Lengthy table referenced here
US20150361494A1-20151217-T00026
Please refer to the end of the specification for access instructions.

Lengthy table referenced here
US20150361494A1-20151217-T00027
Please refer to the end of the specification for access instructions.

Lengthy table referenced here
US20150361494A1-20151217-T00028
Please refer to the end of the specification for access instructions.

Lengthy table referenced here
US20150361494A1-20151217-T00029
Please refer to the end of the specification for access instructions.

Lengthy table referenced here
US20150361494A1-20151217-T00030
Please refer to the end of the specification for access instructions.

Lengthy table referenced here
US20150361494A1-20151217-T00031
Please refer to the end of the specification for access instructions.

Lengthy table referenced here
US20150361494A1-20151217-T00032
Please refer to the end of the specification for access instructions.

Lengthy table referenced here
US20150361494A1-20151217-T00033
Please refer to the end of the specification for access instructions.

Lengthy table referenced here
US20150361494A1-20151217-T00034
Please refer to the end of the specification for access instructions.

Lengthy table referenced here
US20150361494A1-20151217-T00035
Please refer to the end of the specification for access instructions.

Lengthy table referenced here
US20150361494A1-20151217-T00036
Please refer to the end of the specification for access instructions.

Lengthy table referenced here
US20150361494A1-20151217-T00037
Please refer to the end of the specification for access instructions.

Lengthy table referenced here
US20150361494A1-20151217-T00038
Please refer to the end of the specification for access instructions.

Lengthy table referenced here
US20150361494A1-20151217-T00039
Please refer to the end of the specification for access instructions.

Lengthy table referenced here
US20150361494A1-20151217-T00040
Please refer to the end of the specification for access instructions.

Lengthy table referenced here
US20150361494A1-20151217-T00041
Please refer to the end of the specification for access instructions.

Lengthy table referenced here
US20150361494A1-20151217-T00042
Please refer to the end of the specification for access instructions.

Lengthy table referenced here
US20150361494A1-20151217-T00043
Please refer to the end of the specification for access instructions.

Lengthy table referenced here
US20150361494A1-20151217-T00044
Please refer to the end of the specification for access instructions.

Lengthy table referenced here
US20150361494A1-20151217-T00045
Please refer to the end of the specification for access instructions.

Lengthy table referenced here
US20150361494A1-20151217-T00046
Please refer to the end of the specification for access instructions.

Lengthy table referenced here
US20150361494A1-20151217-T00047
Please refer to the end of the specification for access instructions.

Lengthy table referenced here
US20150361494A1-20151217-T00048
Please refer to the end of the specification for access instructions.

Lengthy table referenced here
US20150361494A1-20151217-T00049
Please refer to the end of the specification for access instructions.

Lengthy table referenced here
US20150361494A1-20151217-T00050
Please refer to the end of the specification for access instructions.

Lengthy table referenced here
US20150361494A1-20151217-T00051
Please refer to the end of the specification for access instructions.

Lengthy table referenced here
US20150361494A1-20151217-T00052
Please refer to the end of the specification for access instructions.

Lengthy table referenced here
US20150361494A1-20151217-T00053
Please refer to the end of the specification for access instructions.

Lengthy table referenced here
US20150361494A1-20151217-T00054
Please refer to the end of the specification for access instructions.

Lengthy table referenced here
US20150361494A1-20151217-T00055
Please refer to the end of the specification for access instructions.

Lengthy table referenced here
US20150361494A1-20151217-T00056
Please refer to the end of the specification for access instructions.

Lengthy table referenced here
US20150361494A1-20151217-T00057
Please refer to the end of the specification for access instructions.

Lengthy table referenced here
US20150361494A1-20151217-T00058
Please refer to the end of the specification for access instructions.

Lengthy table referenced here
US20150361494A1-20151217-T00059
Please refer to the end of the specification for access instructions.

Lengthy table referenced here
US20150361494A1-20151217-T00060
Please refer to the end of the specification for access instructions.

Lengthy table referenced here
US20150361494A1-20151217-T00061
Please refer to the end of the specification for access instructions.

Lengthy table referenced here
US20150361494A1-20151217-T00062
Please refer to the end of the specification for access instructions.

Lengthy table referenced here
US20150361494A1-20151217-T00063
Please refer to the end of the specification for access instructions.

Lengthy table referenced here
US20150361494A1-20151217-T00064
Please refer to the end of the specification for access instructions.

Lengthy table referenced here
US20150361494A1-20151217-T00065
Please refer to the end of the specification for access instructions.

Lengthy table referenced here
US20150361494A1-20151217-T00066
Please refer to the end of the specification for access instructions.

Lengthy table referenced here
US20150361494A1-20151217-T00067
Please refer to the end of the specification for access instructions.

Lengthy table referenced here
US20150361494A1-20151217-T00068
Please refer to the end of the specification for access instructions.

Lengthy table referenced here
US20150361494A1-20151217-T00069
Please refer to the end of the specification for access instructions.

Lengthy table referenced here
US20150361494A1-20151217-T00070
Please refer to the end of the specification for access instructions.

Lengthy table referenced here
US20150361494A1-20151217-T00071
Please refer to the end of the specification for access instructions.

Lengthy table referenced here
US20150361494A1-20151217-T00072
Please refer to the end of the specification for access instructions.

Lengthy table referenced here
US20150361494A1-20151217-T00073
Please refer to the end of the specification for access instructions.

Lengthy table referenced here
US20150361494A1-20151217-T00074
Please refer to the end of the specification for access instructions.

Lengthy table referenced here
US20150361494A1-20151217-T00075
Please refer to the end of the specification for access instructions.

Lengthy table referenced here
US20150361494A1-20151217-T00076
Please refer to the end of the specification for access instructions.

Lengthy table referenced here
US20150361494A1-20151217-T00077
Please refer to the end of the specification for access instructions.

Lengthy table referenced here
US20150361494A1-20151217-T00078
Please refer to the end of the specification for access instructions.

Lengthy table referenced here
US20150361494A1-20151217-T00079
Please refer to the end of the specification for access instructions.

Lengthy table referenced here
US20150361494A1-20151217-T00080
Please refer to the end of the specification for access instructions.

Lengthy table referenced here
US20150361494A1-20151217-T00081
Please refer to the end of the specification for access instructions.

Lengthy table referenced here
US20150361494A1-20151217-T00082
Please refer to the end of the specification for access instructions.

Lengthy table referenced here
US20150361494A1-20151217-T00083
Please refer to the end of the specification for access instructions.

Lengthy table referenced here
US20150361494A1-20151217-T00084
Please refer to the end of the specification for access instructions.

Lengthy table referenced here
US20150361494A1-20151217-T00085
Please refer to the end of the specification for access instructions.

Lengthy table referenced here
US20150361494A1-20151217-T00086
Please refer to the end of the specification for access instructions.

Lengthy table referenced here
US20150361494A1-20151217-T00087
Please refer to the end of the specification for access instructions.

Lengthy table referenced here
US20150361494A1-20151217-T00088
Please refer to the end of the specification for access instructions.

Lengthy table referenced here
US20150361494A1-20151217-T00089
Please refer to the end of the specification for access instructions.

Lengthy table referenced here
US20150361494A1-20151217-T00090
Please refer to the end of the specification for access instructions.

Lengthy table referenced here
US20150361494A1-20151217-T00091
Please refer to the end of the specification for access instructions.

Lengthy table referenced here
US20150361494A1-20151217-T00092
Please refer to the end of the specification for access instructions.

Lengthy table referenced here
US20150361494A1-20151217-T00093
Please refer to the end of the specification for access instructions.

Lengthy table referenced here
US20150361494A1-20151217-T00094
Please refer to the end of the specification for access instructions.

Lengthy table referenced here
US20150361494A1-20151217-T00095
Please refer to the end of the specification for access instructions.

Lengthy table referenced here
US20150361494A1-20151217-T00096
Please refer to the end of the specification for access instructions.

Lengthy table referenced here
US20150361494A1-20151217-T00097
Please refer to the end of the specification for access instructions.

Lengthy table referenced here
US20150361494A1-20151217-T00098
Please refer to the end of the specification for access instructions.

Lengthy table referenced here
US20150361494A1-20151217-T00099
Please refer to the end of the specification for access instructions.

Lengthy table referenced here
US20150361494A1-20151217-T00100
Please refer to the end of the specification for access instructions.

Lengthy table referenced here
US20150361494A1-20151217-T00101
Please refer to the end of the specification for access instructions.

Lengthy table referenced here
US20150361494A1-20151217-T00102
Please refer to the end of the specification for access instructions.

Lengthy table referenced here
US20150361494A1-20151217-T00103
Please refer to the end of the specification for access instructions.

Lengthy table referenced here
US20150361494A1-20151217-T00104
Please refer to the end of the specification for access instructions.

Lengthy table referenced here
US20150361494A1-20151217-T00105
Please refer to the end of the specification for access instructions.

Lengthy table referenced here
US20150361494A1-20151217-T00106
Please refer to the end of the specification for access instructions.

Lengthy table referenced here
US20150361494A1-20151217-T00107
Please refer to the end of the specification for access instructions.

Lengthy table referenced here
US20150361494A1-20151217-T00108
Please refer to the end of the specification for access instructions.

Lengthy table referenced here
US20150361494A1-20151217-T00109
Please refer to the end of the specification for access instructions.

Lengthy table referenced here
US20150361494A1-20151217-T00110
Please refer to the end of the specification for access instructions.

Lengthy table referenced here
US20150361494A1-20151217-T00111
Please refer to the end of the specification for access instructions.

Lengthy table referenced here
US20150361494A1-20151217-T00112
Please refer to the end of the specification for access instructions.

Lengthy table referenced here
US20150361494A1-20151217-T00113
Please refer to the end of the specification for access instructions.

Lengthy table referenced here
US20150361494A1-20151217-T00114
Please refer to the end of the specification for access instructions.

Lengthy table referenced here
US20150361494A1-20151217-T00115
Please refer to the end of the specification for access instructions.

Lengthy table referenced here
US20150361494A1-20151217-T00116
Please refer to the end of the specification for access instructions.

Lengthy table referenced here
US20150361494A1-20151217-T00117
Please refer to the end of the specification for access instructions.

Lengthy table referenced here
US20150361494A1-20151217-T00118
Please refer to the end of the specification for access instructions.

Lengthy table referenced here
US20150361494A1-20151217-T00119
Please refer to the end of the specification for access instructions.

Lengthy table referenced here
US20150361494A1-20151217-T00120
Please refer to the end of the specification for access instructions.

Lengthy table referenced here
US20150361494A1-20151217-T00121
Please refer to the end of the specification for access instructions.

Lengthy table referenced here
US20150361494A1-20151217-T00122
Please refer to the end of the specification for access instructions.

Lengthy table referenced here
US20150361494A1-20151217-T00123
Please refer to the end of the specification for access instructions.

Lengthy table referenced here
US20150361494A1-20151217-T00124
Please refer to the end of the specification for access instructions.

Lengthy table referenced here
US20150361494A1-20151217-T00125
Please refer to the end of the specification for access instructions.

Lengthy table referenced here
US20150361494A1-20151217-T00126
Please refer to the end of the specification for access instructions.

Lengthy table referenced here
US20150361494A1-20151217-T00127
Please refer to the end of the specification for access instructions.

Lengthy table referenced here
US20150361494A1-20151217-T00128
Please refer to the end of the specification for access instructions.

Lengthy table referenced here
US20150361494A1-20151217-T00129
Please refer to the end of the specification for access instructions.

Lengthy table referenced here
US20150361494A1-20151217-T00130
Please refer to the end of the specification for access instructions.

Lengthy table referenced here
US20150361494A1-20151217-T00131
Please refer to the end of the specification for access instructions.

Lengthy table referenced here
US20150361494A1-20151217-T00132
Please refer to the end of the specification for access instructions.

Lengthy table referenced here
US20150361494A1-20151217-T00133
Please refer to the end of the specification for access instructions.

Lengthy table referenced here
US20150361494A1-20151217-T00134
Please refer to the end of the specification for access instructions.

Lengthy table referenced here
US20150361494A1-20151217-T00135
Please refer to the end of the specification for access instructions.

Lengthy table referenced here
US20150361494A1-20151217-T00136
Please refer to the end of the specification for access instructions.

Lengthy table referenced here
US20150361494A1-20151217-T00137
Please refer to the end of the specification for access instructions.

Lengthy table referenced here
US20150361494A1-20151217-T00138
Please refer to the end of the specification for access instructions.

Lengthy table referenced here
US20150361494A1-20151217-T00139
Please refer to the end of the specification for access instructions.

Lengthy table referenced here
US20150361494A1-20151217-T00140
Please refer to the end of the specification for access instructions.

Lengthy table referenced here
US20150361494A1-20151217-T00141
Please refer to the end of the specification for access instructions.

Lengthy table referenced here
US20150361494A1-20151217-T00142
Please refer to the end of the specification for access instructions.

Lengthy table referenced here
US20150361494A1-20151217-T00143
Please refer to the end of the specification for access instructions.

Lengthy table referenced here
US20150361494A1-20151217-T00144
Please refer to the end of the specification for access instructions.

Lengthy table referenced here
US20150361494A1-20151217-T00145
Please refer to the end of the specification for access instructions.

Lengthy table referenced here
US20150361494A1-20151217-T00146
Please refer to the end of the specification for access instructions.

Lengthy table referenced here
US20150361494A1-20151217-T00147
Please refer to the end of the specification for access instructions.

Lengthy table referenced here
US20150361494A1-20151217-T00148
Please refer to the end of the specification for access instructions.

Lengthy table referenced here
US20150361494A1-20151217-T00149
Please refer to the end of the specification for access instructions.

Lengthy table referenced here
US20150361494A1-20151217-T00150
Please refer to the end of the specification for access instructions.

Lengthy table referenced here
US20150361494A1-20151217-T00151
Please refer to the end of the specification for access instructions.

Lengthy table referenced here
US20150361494A1-20151217-T00152
Please refer to the end of the specification for access instructions.

Lengthy table referenced here
US20150361494A1-20151217-T00153
Please refer to the end of the specification for access instructions.

Lengthy table referenced here
US20150361494A1-20151217-T00154
Please refer to the end of the specification for access instructions.

Lengthy table referenced here
US20150361494A1-20151217-T00155
Please refer to the end of the specification for access instructions.

Lengthy table referenced here
US20150361494A1-20151217-T00156
Please refer to the end of the specification for access instructions.

Lengthy table referenced here
US20150361494A1-20151217-T00157
Please refer to the end of the specification for access instructions.

Lengthy table referenced here
US20150361494A1-20151217-T00158
Please refer to the end of the specification for access instructions.

Lengthy table referenced here
US20150361494A1-20151217-T00159
Please refer to the end of the specification for access instructions.

Lengthy table referenced here
US20150361494A1-20151217-T00160
Please refer to the end of the specification for access instructions.

Lengthy table referenced here
US20150361494A1-20151217-T00161
Please refer to the end of the specification for access instructions.

Lengthy table referenced here
US20150361494A1-20151217-T00162
Please refer to the end of the specification for access instructions.

Lengthy table referenced here
US20150361494A1-20151217-T00163
Please refer to the end of the specification for access instructions.

Lengthy table referenced here
US20150361494A1-20151217-T00164
Please refer to the end of the specification for access instructions.

Lengthy table referenced here
US20150361494A1-20151217-T00165
Please refer to the end of the specification for access instructions.

Lengthy table referenced here
US20150361494A1-20151217-T00166
Please refer to the end of the specification for access instructions.

Lengthy table referenced here
US20150361494A1-20151217-T00167
Please refer to the end of the specification for access instructions.

Lengthy table referenced here
US20150361494A1-20151217-T00168
Please refer to the end of the specification for access instructions.

Lengthy table referenced here
US20150361494A1-20151217-T00169
Please refer to the end of the specification for access instructions.

Lengthy table referenced here
US20150361494A1-20151217-T00170
Please refer to the end of the specification for access instructions.

Lengthy table referenced here
US20150361494A1-20151217-T00171
Please refer to the end of the specification for access instructions.

Lengthy table referenced here
US20150361494A1-20151217-T00172
Please refer to the end of the specification for access instructions.

Lengthy table referenced here
US20150361494A1-20151217-T00173
Please refer to the end of the specification for access instructions.

Lengthy table referenced here
US20150361494A1-20151217-T00174
Please refer to the end of the specification for access instructions.

Lengthy table referenced here
US20150361494A1-20151217-T00175
Please refer to the end of the specification for access instructions.

Lengthy table referenced here
US20150361494A1-20151217-T00176
Please refer to the end of the specification for access instructions.

Lengthy table referenced here
US20150361494A1-20151217-T00177
Please refer to the end of the specification for access instructions.

Lengthy table referenced here
US20150361494A1-20151217-T00178
Please refer to the end of the specification for access instructions.

Lengthy table referenced here
US20150361494A1-20151217-T00179
Please refer to the end of the specification for access instructions.

Lengthy table referenced here
US20150361494A1-20151217-T00180
Please refer to the end of the specification for access instructions.

Lengthy table referenced here
US20150361494A1-20151217-T00181
Please refer to the end of the specification for access instructions.

Lengthy table referenced here
US20150361494A1-20151217-T00182
Please refer to the end of the specification for access instructions.

Lengthy table referenced here
US20150361494A1-20151217-T00183
Please refer to the end of the specification for access instructions.

Lengthy table referenced here
US20150361494A1-20151217-T00184
Please refer to the end of the specification for access instructions.

Lengthy table referenced here
US20150361494A1-20151217-T00185
Please refer to the end of the specification for access instructions.

Lengthy table referenced here
US20150361494A1-20151217-T00186
Please refer to the end of the specification for access instructions.

Lengthy table referenced here
US20150361494A1-20151217-T00187
Please refer to the end of the specification for access instructions.

Lengthy table referenced here
US20150361494A1-20151217-T00188
Please refer to the end of the specification for access instructions.

Lengthy table referenced here
US20150361494A1-20151217-T00189
Please refer to the end of the specification for access instructions.

Lengthy table referenced here
US20150361494A1-20151217-T00190
Please refer to the end of the specification for access instructions.

Lengthy table referenced here
US20150361494A1-20151217-T00191
Please refer to the end of the specification for access instructions.

Lengthy table referenced here
US20150361494A1-20151217-T00192
Please refer to the end of the specification for access instructions.

Lengthy table referenced here
US20150361494A1-20151217-T00193
Please refer to the end of the specification for access instructions.

Lengthy table referenced here
US20150361494A1-20151217-T00194
Please refer to the end of the specification for access instructions.

Lengthy table referenced here
US20150361494A1-20151217-T00195
Please refer to the end of the specification for access instructions.

Lengthy table referenced here
US20150361494A1-20151217-T00196
Please refer to the end of the specification for access instructions.

Lengthy table referenced here
US20150361494A1-20151217-T00197
Please refer to the end of the specification for access instructions.

Lengthy table referenced here
US20150361494A1-20151217-T00198
Please refer to the end of the specification for access instructions.

Lengthy table referenced here
US20150361494A1-20151217-T00199
Please refer to the end of the specification for access instructions.

Lengthy table referenced here
US20150361494A1-20151217-T00200
Please refer to the end of the specification for access instructions.

Lengthy table referenced here
US20150361494A1-20151217-T00201
Please refer to the end of the specification for access instructions.

Lengthy table referenced here
US20150361494A1-20151217-T00202
Please refer to the end of the specification for access instructions.

Lengthy table referenced here
US20150361494A1-20151217-T00203
Please refer to the end of the specification for access instructions.

Lengthy table referenced here
US20150361494A1-20151217-T00204
Please refer to the end of the specification for access instructions.

Lengthy table referenced here
US20150361494A1-20151217-T00205
Please refer to the end of the specification for access instructions.

Lengthy table referenced here
US20150361494A1-20151217-T00206
Please refer to the end of the specification for access instructions.

Lengthy table referenced here
US20150361494A1-20151217-T00207
Please refer to the end of the specification for access instructions.

Lengthy table referenced here
US20150361494A1-20151217-T00208
Please refer to the end of the specification for access instructions.

Lengthy table referenced here
US20150361494A1-20151217-T00209
Please refer to the end of the specification for access instructions.

Lengthy table referenced here
US20150361494A1-20151217-T00210
Please refer to the end of the specification for access instructions.

Lengthy table referenced here
US20150361494A1-20151217-T00211
Please refer to the end of the specification for access instructions.

Lengthy table referenced here
US20150361494A1-20151217-T00212
Please refer to the end of the specification for access instructions.

Lengthy table referenced here
US20150361494A1-20151217-T00213
Please refer to the end of the specification for access instructions.

Lengthy table referenced here
US20150361494A1-20151217-T00214
Please refer to the end of the specification for access instructions.

Lengthy table referenced here
US20150361494A1-20151217-T00215
Please refer to the end of the specification for access instructions.

Lengthy table referenced here
US20150361494A1-20151217-T00216
Please refer to the end of the specification for access instructions.

Lengthy table referenced here
US20150361494A1-20151217-T00217
Please refer to the end of the specification for access instructions.

Lengthy table referenced here
US20150361494A1-20151217-T00218
Please refer to the end of the specification for access instructions.

Lengthy table referenced here
US20150361494A1-20151217-T00219
Please refer to the end of the specification for access instructions.

Lengthy table referenced here
US20150361494A1-20151217-T00220
Please refer to the end of the specification for access instructions.

Lengthy table referenced here
US20150361494A1-20151217-T00221
Please refer to the end of the specification for access instructions.

Lengthy table referenced here
US20150361494A1-20151217-T00222
Please refer to the end of the specification for access instructions.

Lengthy table referenced here
US20150361494A1-20151217-T00223
Please refer to the end of the specification for access instructions.

Lengthy table referenced here
US20150361494A1-20151217-T00224
Please refer to the end of the specification for access instructions.

Lengthy table referenced here
US20150361494A1-20151217-T00225
Please refer to the end of the specification for access instructions.

Lengthy table referenced here
US20150361494A1-20151217-T00226
Please refer to the end of the specification for access instructions.

Lengthy table referenced here
US20150361494A1-20151217-T00227
Please refer to the end of the specification for access instructions.

Lengthy table referenced here
US20150361494A1-20151217-T00228
Please refer to the end of the specification for access instructions.

Lengthy table referenced here
US20150361494A1-20151217-T00229
Please refer to the end of the specification for access instructions.

Lengthy table referenced here
US20150361494A1-20151217-T00230
Please refer to the end of the specification for access instructions.

Lengthy table referenced here
US20150361494A1-20151217-T00231
Please refer to the end of the specification for access instructions.

Lengthy table referenced here
US20150361494A1-20151217-T00232
Please refer to the end of the specification for access instructions.

Lengthy table referenced here
US20150361494A1-20151217-T00233
Please refer to the end of the specification for access instructions.

Lengthy table referenced here
US20150361494A1-20151217-T00234
Please refer to the end of the specification for access instructions.

Lengthy table referenced here
US20150361494A1-20151217-T00235
Please refer to the end of the specification for access instructions.

Lengthy table referenced here
US20150361494A1-20151217-T00236
Please refer to the end of the specification for access instructions.

Lengthy table referenced here
US20150361494A1-20151217-T00237
Please refer to the end of the specification for access instructions.

Lengthy table referenced here
US20150361494A1-20151217-T00238
Please refer to the end of the specification for access instructions.

Lengthy table referenced here
US20150361494A1-20151217-T00239
Please refer to the end of the specification for access instructions.

Lengthy table referenced here
US20150361494A1-20151217-T00240
Please refer to the end of the specification for access instructions.

Lengthy table referenced here
US20150361494A1-20151217-T00241
Please refer to the end of the specification for access instructions.

Lengthy table referenced here
US20150361494A1-20151217-T00242
Please refer to the end of the specification for access instructions.

Lengthy table referenced here
US20150361494A1-20151217-T00243
Please refer to the end of the specification for access instructions.

Lengthy table referenced here
US20150361494A1-20151217-T00244
Please refer to the end of the specification for access instructions.

Lengthy table referenced here
US20150361494A1-20151217-T00245
Please refer to the end of the specification for access instructions.

Lengthy table referenced here
US20150361494A1-20151217-T00246
Please refer to the end of the specification for access instructions.

Lengthy table referenced here
US20150361494A1-20151217-T00247
Please refer to the end of the specification for access instructions.

Lengthy table referenced here
US20150361494A1-20151217-T00248
Please refer to the end of the specification for access instructions.

Lengthy table referenced here
US20150361494A1-20151217-T00249
Please refer to the end of the specification for access instructions.

Lengthy table referenced here
US20150361494A1-20151217-T00250
Please refer to the end of the specification for access instructions.

Lengthy table referenced here
US20150361494A1-20151217-T00251
Please refer to the end of the specification for access instructions.

Lengthy table referenced here
US20150361494A1-20151217-T00252
Please refer to the end of the specification for access instructions.

Lengthy table referenced here
US20150361494A1-20151217-T00253
Please refer to the end of the specification for access instructions.

Lengthy table referenced here
US20150361494A1-20151217-T00254
Please refer to the end of the specification for access instructions.

Lengthy table referenced here
US20150361494A1-20151217-T00255
Please refer to the end of the specification for access instructions.

Lengthy table referenced here
US20150361494A1-20151217-T00256
Please refer to the end of the specification for access instructions.

Lengthy table referenced here
US20150361494A1-20151217-T00257
Please refer to the end of the specification for access instructions.

Lengthy table referenced here
US20150361494A1-20151217-T00258
Please refer to the end of the specification for access instructions.

Lengthy table referenced here
US20150361494A1-20151217-T00259
Please refer to the end of the specification for access instructions.

Lengthy table referenced here
US20150361494A1-20151217-T00260
Please refer to the end of the specification for access instructions.

Lengthy table referenced here
US20150361494A1-20151217-T00261
Please refer to the end of the specification for access instructions.

Lengthy table referenced here
US20150361494A1-20151217-T00262
Please refer to the end of the specification for access instructions.

Lengthy table referenced here
US20150361494A1-20151217-T00263
Please refer to the end of the specification for access instructions.

Lengthy table referenced here
US20150361494A1-20151217-T00264
Please refer to the end of the specification for access instructions.

Lengthy table referenced here
US20150361494A1-20151217-T00265
Please refer to the end of the specification for access instructions.

Lengthy table referenced here
US20150361494A1-20151217-T00266
Please refer to the end of the specification for access instructions.

Lengthy table referenced here
US20150361494A1-20151217-T00267
Please refer to the end of the specification for access instructions.

Lengthy table referenced here
US20150361494A1-20151217-T00268
Please refer to the end of the specification for access instructions.

Lengthy table referenced here
US20150361494A1-20151217-T00269
Please refer to the end of the specification for access instructions.

Lengthy table referenced here
US20150361494A1-20151217-T00270
Please refer to the end of the specification for access instructions.

Lengthy table referenced here
US20150361494A1-20151217-T00271
Please refer to the end of the specification for access instructions.

Lengthy table referenced here
US20150361494A1-20151217-T00272
Please refer to the end of the specification for access instructions.

Lengthy table referenced here
US20150361494A1-20151217-T00273
Please refer to the end of the specification for access instructions.

Lengthy table referenced here
US20150361494A1-20151217-T00274
Please refer to the end of the specification for access instructions.

Lengthy table referenced here
US20150361494A1-20151217-T00275
Please refer to the end of the specification for access instructions.

Lengthy table referenced here
US20150361494A1-20151217-T00276
Please refer to the end of the specification for access instructions.

Lengthy table referenced here
US20150361494A1-20151217-T00277
Please refer to the end of the specification for access instructions.

Lengthy table referenced here
US20150361494A1-20151217-T00278
Please refer to the end of the specification for access instructions.

Lengthy table referenced here
US20150361494A1-20151217-T00279
Please refer to the end of the specification for access instructions.

Lengthy table referenced here
US20150361494A1-20151217-T00280
Please refer to the end of the specification for access instructions.

Lengthy table referenced here
US20150361494A1-20151217-T00281
Please refer to the end of the specification for access instructions.

Lengthy table referenced here
US20150361494A1-20151217-T00282
Please refer to the end of the specification for access instructions.

Lengthy table referenced here
US20150361494A1-20151217-T00283
Please refer to the end of the specification for access instructions.

Lengthy table referenced here
US20150361494A1-20151217-T00284
Please refer to the end of the specification for access instructions.

Lengthy table referenced here
US20150361494A1-20151217-T00285
Please refer to the end of the specification for access instructions.

Lengthy table referenced here
US20150361494A1-20151217-T00286
Please refer to the end of the specification for access instructions.

Lengthy table referenced here
US20150361494A1-20151217-T00287
Please refer to the end of the specification for access instructions.

Lengthy table referenced here
US20150361494A1-20151217-T00288
Please refer to the end of the specification for access instructions.

Lengthy table referenced here
US20150361494A1-20151217-T00289
Please refer to the end of the specification for access instructions.

Lengthy table referenced here
US20150361494A1-20151217-T00290
Please refer to the end of the specification for access instructions.

Lengthy table referenced here
US20150361494A1-20151217-T00291
Please refer to the end of the specification for access instructions.

Lengthy table referenced here
US20150361494A1-20151217-T00292
Please refer to the end of the specification for access instructions.

Lengthy table referenced here
US20150361494A1-20151217-T00293
Please refer to the end of the specification for access instructions.

Lengthy table referenced here
US20150361494A1-20151217-T00294
Please refer to the end of the specification for access instructions.

Lengthy table referenced here
US20150361494A1-20151217-T00295
Please refer to the end of the specification for access instructions.

Lengthy table referenced here
US20150361494A1-20151217-T00296
Please refer to the end of the specification for access instructions.

Lengthy table referenced here
US20150361494A1-20151217-T00297
Please refer to the end of the specification for access instructions.

Lengthy table referenced here
US20150361494A1-20151217-T00298
Please refer to the end of the specification for access instructions.

Lengthy table referenced here
US20150361494A1-20151217-T00299
Please refer to the end of the specification for access instructions.

Lengthy table referenced here
US20150361494A1-20151217-T00300
Please refer to the end of the specification for access instructions.

Lengthy table referenced here
US20150361494A1-20151217-T00301
Please refer to the end of the specification for access instructions.

Lengthy table referenced here
US20150361494A1-20151217-T00302
Please refer to the end of the specification for access instructions.

Lengthy table referenced here
US20150361494A1-20151217-T00303
Please refer to the end of the specification for access instructions.

Lengthy table referenced here
US20150361494A1-20151217-T00304
Please refer to the end of the specification for access instructions.

Lengthy table referenced here
US20150361494A1-20151217-T00305
Please refer to the end of the specification for access instructions.

Lengthy table referenced here
US20150361494A1-20151217-T00306
Please refer to the end of the specification for access instructions.

Lengthy table referenced here
US20150361494A1-20151217-T00307
Please refer to the end of the specification for access instructions.

Lengthy table referenced here
US20150361494A1-20151217-T00308
Please refer to the end of the specification for access instructions.

Lengthy table referenced here
US20150361494A1-20151217-T00309
Please refer to the end of the specification for access instructions.

Lengthy table referenced here
US20150361494A1-20151217-T00310
Please refer to the end of the specification for access instructions.

Lengthy table referenced here
US20150361494A1-20151217-T00311
Please refer to the end of the specification for access instructions.

Lengthy table referenced here
US20150361494A1-20151217-T00312
Please refer to the end of the specification for access instructions.

Lengthy table referenced here
US20150361494A1-20151217-T00313
Please refer to the end of the specification for access instructions.

Lengthy table referenced here
US20150361494A1-20151217-T00314
Please refer to the end of the specification for access instructions.

Lengthy table referenced here
US20150361494A1-20151217-T00315
Please refer to the end of the specification for access instructions.

Lengthy table referenced here
US20150361494A1-20151217-T00316
Please refer to the end of the specification for access instructions.

Lengthy table referenced here
US20150361494A1-20151217-T00317
Please refer to the end of the specification for access instructions.

Lengthy table referenced here
US20150361494A1-20151217-T00318
Please refer to the end of the specification for access instructions.

Lengthy table referenced here
US20150361494A1-20151217-T00319
Please refer to the end of the specification for access instructions.

Lengthy table referenced here
US20150361494A1-20151217-T00320
Please refer to the end of the specification for access instructions.

Lengthy table referenced here
US20150361494A1-20151217-T00321
Please refer to the end of the specification for access instructions.

Lengthy table referenced here
US20150361494A1-20151217-T00322
Please refer to the end of the specification for access instructions.

Lengthy table referenced here
US20150361494A1-20151217-T00323
Please refer to the end of the specification for access instructions.

Lengthy table referenced here
US20150361494A1-20151217-T00324
Please refer to the end of the specification for access instructions.

Lengthy table referenced here
US20150361494A1-20151217-T00325
Please refer to the end of the specification for access instructions.

Lengthy table referenced here
US20150361494A1-20151217-T00326
Please refer to the end of the specification for access instructions.

Lengthy table referenced here
US20150361494A1-20151217-T00327
Please refer to the end of the specification for access instructions.

Lengthy table referenced here
US20150361494A1-20151217-T00328
Please refer to the end of the specification for access instructions.

Lengthy table referenced here
US20150361494A1-20151217-T00329
Please refer to the end of the specification for access instructions.

Lengthy table referenced here
US20150361494A1-20151217-T00330
Please refer to the end of the specification for access instructions.

Lengthy table referenced here
US20150361494A1-20151217-T00331
Please refer to the end of the specification for access instructions.

Lengthy table referenced here
US20150361494A1-20151217-T00332
Please refer to the end of the specification for access instructions.

Lengthy table referenced here
US20150361494A1-20151217-T00333
Please refer to the end of the specification for access instructions.

Lengthy table referenced here
US20150361494A1-20151217-T00334
Please refer to the end of the specification for access instructions.

Lengthy table referenced here
US20150361494A1-20151217-T00335
Please refer to the end of the specification for access instructions.

Lengthy table referenced here
US20150361494A1-20151217-T00336
Please refer to the end of the specification for access instructions.

Lengthy table referenced here
US20150361494A1-20151217-T00337
Please refer to the end of the specification for access instructions.

Lengthy table referenced here
US20150361494A1-20151217-T00338
Please refer to the end of the specification for access instructions.

Lengthy table referenced here
US20150361494A1-20151217-T00339
Please refer to the end of the specification for access instructions.

Lengthy table referenced here
US20150361494A1-20151217-T00340
Please refer to the end of the specification for access instructions.

Lengthy table referenced here
US20150361494A1-20151217-T00341
Please refer to the end of the specification for access instructions.

Lengthy table referenced here
US20150361494A1-20151217-T00342
Please refer to the end of the specification for access instructions.

Lengthy table referenced here
US20150361494A1-20151217-T00343
Please refer to the end of the specification for access instructions.

Lengthy table referenced here
US20150361494A1-20151217-T00344
Please refer to the end of the specification for access instructions.

Lengthy table referenced here
US20150361494A1-20151217-T00345
Please refer to the end of the specification for access instructions.

Lengthy table referenced here
US20150361494A1-20151217-T00346
Please refer to the end of the specification for access instructions.

Lengthy table referenced here
US20150361494A1-20151217-T00347
Please refer to the end of the specification for access instructions.

Lengthy table referenced here
US20150361494A1-20151217-T00348
Please refer to the end of the specification for access instructions.

Lengthy table referenced here
US20150361494A1-20151217-T00349
Please refer to the end of the specification for access instructions.

Lengthy table referenced here
US20150361494A1-20151217-T00350
Please refer to the end of the specification for access instructions.

Lengthy table referenced here
US20150361494A1-20151217-T00351
Please refer to the end of the specification for access instructions.

Lengthy table referenced here
US20150361494A1-20151217-T00352
Please refer to the end of the specification for access instructions.

Lengthy table referenced here
US20150361494A1-20151217-T00353
Please refer to the end of the specification for access instructions.

Lengthy table referenced here
US20150361494A1-20151217-T00354
Please refer to the end of the specification for access instructions.

Lengthy table referenced here
US20150361494A1-20151217-T00355
Please refer to the end of the specification for access instructions.

Lengthy table referenced here
US20150361494A1-20151217-T00356
Please refer to the end of the specification for access instructions.

Lengthy table referenced here
US20150361494A1-20151217-T00357
Please refer to the end of the specification for access instructions.

Lengthy table referenced here
US20150361494A1-20151217-T00358
Please refer to the end of the specification for access instructions.

Lengthy table referenced here
US20150361494A1-20151217-T00359
Please refer to the end of the specification for access instructions.

Lengthy table referenced here
US20150361494A1-20151217-T00360
Please refer to the end of the specification for access instructions.

Lengthy table referenced here
US20150361494A1-20151217-T00361
Please refer to the end of the specification for access instructions.

Lengthy table referenced here
US20150361494A1-20151217-T00362
Please refer to the end of the specification for access instructions.

Lengthy table referenced here
US20150361494A1-20151217-T00363
Please refer to the end of the specification for access instructions.

Lengthy table referenced here
US20150361494A1-20151217-T00364
Please refer to the end of the specification for access instructions.

Lengthy table referenced here
US20150361494A1-20151217-T00365
Please refer to the end of the specification for access instructions.

Lengthy table referenced here
US20150361494A1-20151217-T00366
Please refer to the end of the specification for access instructions.

Lengthy table referenced here
US20150361494A1-20151217-T00367
Please refer to the end of the specification for access instructions.

Lengthy table referenced here
US20150361494A1-20151217-T00368
Please refer to the end of the specification for access instructions.

Lengthy table referenced here
US20150361494A1-20151217-T00369
Please refer to the end of the specification for access instructions.

Lengthy table referenced here
US20150361494A1-20151217-T00370
Please refer to the end of the specification for access instructions.

Lengthy table referenced here
US20150361494A1-20151217-T00371
Please refer to the end of the specification for access instructions.

Lengthy table referenced here
US20150361494A1-20151217-T00372
Please refer to the end of the specification for access instructions.

Lengthy table referenced here
US20150361494A1-20151217-T00373
Please refer to the end of the specification for access instructions.

Lengthy table referenced here
US20150361494A1-20151217-T00374
Please refer to the end of the specification for access instructions.

Lengthy table referenced here
US20150361494A1-20151217-T00375
Please refer to the end of the specification for access instructions.

Lengthy table referenced here
US20150361494A1-20151217-T00376
Please refer to the end of the specification for access instructions.

Lengthy table referenced here
US20150361494A1-20151217-T00377
Please refer to the end of the specification for access instructions.

Lengthy table referenced here
US20150361494A1-20151217-T00378
Please refer to the end of the specification for access instructions.

Lengthy table referenced here
US20150361494A1-20151217-T00379
Please refer to the end of the specification for access instructions.

Lengthy table referenced here
US20150361494A1-20151217-T00380
Please refer to the end of the specification for access instructions.

Lengthy table referenced here
US20150361494A1-20151217-T00381
Please refer to the end of the specification for access instructions.

Lengthy table referenced here
US20150361494A1-20151217-T00382
Please refer to the end of the specification for access instructions.

Lengthy table referenced here
US20150361494A1-20151217-T00383
Please refer to the end of the specification for access instructions.

Lengthy table referenced here
US20150361494A1-20151217-T00384
Please refer to the end of the specification for access instructions.

Lengthy table referenced here
US20150361494A1-20151217-T00385
Please refer to the end of the specification for access instructions.

Lengthy table referenced here
US20150361494A1-20151217-T00386
Please refer to the end of the specification for access instructions.

Lengthy table referenced here
US20150361494A1-20151217-T00387
Please refer to the end of the specification for access instructions.

Lengthy table referenced here
US20150361494A1-20151217-T00388
Please refer to the end of the specification for access instructions.

Lengthy table referenced here
US20150361494A1-20151217-T00389
Please refer to the end of the specification for access instructions.

Lengthy table referenced here
US20150361494A1-20151217-T00390
Please refer to the end of the specification for access instructions.

Lengthy table referenced here
US20150361494A1-20151217-T00391
Please refer to the end of the specification for access instructions.

Lengthy table referenced here
US20150361494A1-20151217-T00392
Please refer to the end of the specification for access instructions.

Lengthy table referenced here
US20150361494A1-20151217-T00393
Please refer to the end of the specification for access instructions.

Lengthy table referenced here
US20150361494A1-20151217-T00394
Please refer to the end of the specification for access instructions.

Lengthy table referenced here
US20150361494A1-20151217-T00395
Please refer to the end of the specification for access instructions.

Lengthy table referenced here
US20150361494A1-20151217-T00396
Please refer to the end of the specification for access instructions.

Lengthy table referenced here
US20150361494A1-20151217-T00397
Please refer to the end of the specification for access instructions.

Lengthy table referenced here
US20150361494A1-20151217-T00398
Please refer to the end of the specification for access instructions.

Lengthy table referenced here
US20150361494A1-20151217-T00399
Please refer to the end of the specification for access instructions.

Lengthy table referenced here
US20150361494A1-20151217-T00400
Please refer to the end of the specification for access instructions.

Lengthy table referenced here
US20150361494A1-20151217-T00401
Please refer to the end of the specification for access instructions.

Lengthy table referenced here
US20150361494A1-20151217-T00402
Please refer to the end of the specification for access instructions.

Lengthy table referenced here
US20150361494A1-20151217-T00403
Please refer to the end of the specification for access instructions.

Lengthy table referenced here
US20150361494A1-20151217-T00404
Please refer to the end of the specification for access instructions.

Lengthy table referenced here
US20150361494A1-20151217-T00405
Please refer to the end of the specification for access instructions.

Lengthy table referenced here
US20150361494A1-20151217-T00406
Please refer to the end of the specification for access instructions.

Lengthy table referenced here
US20150361494A1-20151217-T00407
Please refer to the end of the specification for access instructions.

Lengthy table referenced here
US20150361494A1-20151217-T00408
Please refer to the end of the specification for access instructions.

Lengthy table referenced here
US20150361494A1-20151217-T00409
Please refer to the end of the specification for access instructions.

Lengthy table referenced here
US20150361494A1-20151217-T00410
Please refer to the end of the specification for access instructions.

Lengthy table referenced here
US20150361494A1-20151217-T00411
Please refer to the end of the specification for access instructions.

Lengthy table referenced here
US20150361494A1-20151217-T00412
Please refer to the end of the specification for access instructions.

Lengthy table referenced here
US20150361494A1-20151217-T00413
Please refer to the end of the specification for access instructions.

Lengthy table referenced here
US20150361494A1-20151217-T00414
Please refer to the end of the specification for access instructions.

Lengthy table referenced here
US20150361494A1-20151217-T00415
Please refer to the end of the specification for access instructions.

Lengthy table referenced here
US20150361494A1-20151217-T00416
Please refer to the end of the specification for access instructions.

Lengthy table referenced here
US20150361494A1-20151217-T00417
Please refer to the end of the specification for access instructions.

Lengthy table referenced here
US20150361494A1-20151217-T00418
Please refer to the end of the specification for access instructions.

Lengthy table referenced here
US20150361494A1-20151217-T00419
Please refer to the end of the specification for access instructions.

Lengthy table referenced here
US20150361494A1-20151217-T00420
Please refer to the end of the specification for access instructions.

Lengthy table referenced here
US20150361494A1-20151217-T00421
Please refer to the end of the specification for access instructions.

Lengthy table referenced here
US20150361494A1-20151217-T00422
Please refer to the end of the specification for access instructions.

Lengthy table referenced here
US20150361494A1-20151217-T00423
Please refer to the end of the specification for access instructions.

Lengthy table referenced here
US20150361494A1-20151217-T00424
Please refer to the end of the specification for access instructions.

Lengthy table referenced here
US20150361494A1-20151217-T00425
Please refer to the end of the specification for access instructions.

Lengthy table referenced here
US20150361494A1-20151217-T00426
Please refer to the end of the specification for access instructions.

Lengthy table referenced here
US20150361494A1-20151217-T00427
Please refer to the end of the specification for access instructions.

Lengthy table referenced here
US20150361494A1-20151217-T00428
Please refer to the end of the specification for access instructions.

Lengthy table referenced here
US20150361494A1-20151217-T00429
Please refer to the end of the specification for access instructions.

Lengthy table referenced here
US20150361494A1-20151217-T00430
Please refer to the end of the specification for access instructions.

Lengthy table referenced here
US20150361494A1-20151217-T00431
Please refer to the end of the specification for access instructions.

Lengthy table referenced here
US20150361494A1-20151217-T00432
Please refer to the end of the specification for access instructions.

Lengthy table referenced here
US20150361494A1-20151217-T00433
Please refer to the end of the specification for access instructions.

LENGTHY TABLES
The patent application contains a lengthy table section. A copy of the table is available in electronic form from the USPTO web site (http://seqdata.uspto.gov/?pageRequest=docDetail&DocID=US20150361494A1). An electronic copy of the table will also be available from the USPTO upon request and payment of the fee set forth in 37 CFR 1.19(b)(3).

1.-30. (canceled)

31. A method for use in treating an endometriosis related condition in a patient, said method comprising selecting at least one of a patient at increased risk of existence or predisposition of an endometriosis related condition and having at least one increased risk endometriosis genetic marker in the DNA of said patient and a patient at decreased risk of existence or predisposition of an endometriosis related condition and having at least one decreased risk endometriosis genetic marker in the DNA of said patient, and applying a therapeutic that at least partially compensates for an endometriosis related condition to said patient.

32. The method of claim 31, wherein said patient is asymptomatic of an endometriosis condition.

33. The method of claim 31, wherein said endometriosis related condition existence or predisposition increased risk therapeutic application further defines treating said patient with at least one of a medical device, a pharmaceutical, and a medical device and a pharmaceutical.

34. The method of claim 31, wherein said endometriosis related condition existence or predisposition increased risk therapeutic further defines at least one of a protein, a protein fragment, a peptide, an antibody, a nucleic acid, and a mimetic which modulates the function of a target gene.

35. The method of claim 31, wherein said method further includes the step of recording at least one non-genetic endometriosis related clinical factor of said patient.

36. The method of claim 35, wherein said non-genetic endometriosis related clinical factor defines at least one of age at menarche and BMI (Body Mass Index).

37. The method of claim 31, wherein said endometriosis genetic marker defines the minor allele of at least one SNP selected from the group of SEQ ID No:1 through SEQ ID No:156 and SEQ ID No:158 through SEQ ID No:656.

38. The method of claim 37, wherein said increased risk endometriosis genetic marker defines the minor allele of at least one SNP selected from the group of SEQ ID NOs: 001-007, 019-027, 036, 038, 047, 050, 056, 059, 060, 062, 063, 068, 070-073, 075, 076, 085, 088, 092, 093, 099, 104, 106, 109, 110-112, 114-116, 118-125, 127-131, 135-137, 138, 142, 143, 150, 151, 155, 156, 158, 159, 161-164, 166, 167, 169, 171, 173, 176, 179-191, 208-213, 219, 222-226, 232, 233, 234-242, 244-251, 245, 253, 255-257, 264, 271, 272, 274-277, 282, 284-290, 292, 293, 300-305, 307-311, 313, 314, 317-323, 327-329, 332-340, 342-350, 352, 355-361, 365-367, 371-378, 380-383, 385-387, 391-393, 398, 400, 402, 403, 407-409, 411-416, 418, 421, 425, 429, 431-436, 438, 440, 444, 446-453, 455-457, 459, 461-463, 465-467, 470, 472, 473, 477-479, 483-485, 499, 500-502, 504-506, 509-511, 519, 526, 530-532, 534, 535, 541, 543-546, 548-551, 553, 556-558, 560-566, 569, 572-576, 578-580, 587-593, 598, 600, 601, 603-606, 609-613, 615, 616, 618, 620, 623, 624, 635, 643, 644, 647, 649, and wherein said decreased risk endometriosis genetic marker defines the minor allele of at least one SNP selected from the group of SEQ ID NOs: 008-018, 028-035, 037, 039-046, 048, 049, 051-055, 057, 058, 061, 064-067, 069, 074, 077-084, 086, 087, 089-091, 094-098, 100-103, 105, 107, 108, 113, 117, 126, 132-134, 139-141, 144-149, 152-154, 157, 160, 165, 168, 170, 172, 174, 175, 177, 178, 192, 193-207, 214-218, 220, 221, 227-231, 243, 252, 254, 258-263, 265-270, 273, 278-281, 283, 291, 294-299, 306, 312, 315, 316, 324-326, 330, 331, 341, 351, 353, 354, 362-364, 368-370, 379, 384, 388-390, 394-397, 399, 401, 404-406, 410, 417, 419, 420, 422-424, 426-428, 430, 437, 439, 441-443, 445, 454, 458, 460, 464, 468, 469, 471, 474-476, 480-482, 486-498, 503, 507, 508, 512-518, 520-525, 527-529, 533, 536-540, 542, 547, 552, 554, 555, 559, 567, 568, 570, 571, 577, 581, 582-586, 594-597, 599, 602, 607, 608, 614, 617, 619, 621, 622, 625-634, 636-642, 645, 646, 648, 650-656.

39. A method comprising selecting a patient at altered risk of an endometriosis related condition and having at least one endometriosis altered risk genetic marker in said patient, and applying to said patient a therapeutic that at least partially compensates for an endometriosis related condition.

40. The method of claim 39, wherein said patient is asymptomatic of an endometriosis condition.

41. The method of claim 39, wherein said therapeutic application further defines treating said patient with at least one of a medical device, a pharmaceutical, and a medical device and a pharmaceutical.

42. The method of claim 39, wherein said therapeutic further defines at least one of a protein, a protein fragment, a peptide, an antibody, a nucleic acid, and a mimetic which modulates the function of a target gene.

43. The method of claim 39, wherein said method further includes the step of recording at least one non-genetic endometriosis related clinical factor of said patient.

44. The method of claim 43, wherein said non-genetic endometriosis related clinical factor defines at least one of age at menarche and BMI (Body Mass Index).

45. The method of claim 39, wherein said endometriosis altered risk genetic marker defines the minor allele of at least one SNP selected from the group of SEQ ID No:1 through SEQ ID No:156 and SEQ ID No:158 through SEQ ID No:656.

46. The method of claim 45, wherein said altered risk genetic marker defines at least one of an increased risk endometriosis genetic marker defining the minor allele of at least one SNP selected from the group of SEQ ID NOs: 001-007, 019-027, 036, 038, 047, 050, 056, 059, 060, 062, 063, 068, 070-073, 075, 076, 085, 088, 092, 093, 099, 104, 106, 109, 110-112, 114-116, 118-125, 127-131, 135-137, 138, 142, 143, 150, 151, 155, 156, 158, 159, 161-164, 166, 167, 169, 171, 173, 176, 179-191, 208-213, 219, 222-226, 232, 233, 234-242, 244-251, 245, 253, 255-257, 264, 271, 272, 274-277, 282, 284-290, 292, 293, 300-305, 307-311, 313, 314, 317-323, 327-329, 332-340, 342-350, 352, 355-361, 365-367, 371-378, 380-383, 385-387, 391-393, 398, 400, 402, 403, 407-409, 411-416, 418, 421, 425, 429, 431-436, 438, 440, 444, 446-453, 455-457, 459, 461-463, 465-467, 470, 472, 473, 477-479, 483-485, 499, 500-502, 504-506, 509-511, 519, 526, 530-532, 534, 535, 541, 543-546, 548-551, 553, 556-558, 560-566, 569, 572-576, 578-580, 587-593, 598, 600, 601, 603-606, 609-613, 615, 616, 618, 620, 623, 624, 635, 643, 644, 647, 649, and a decreased risk endometriosis genetic marker defining the minor allele of at least one SNP selected from the group of SEQ ID NOs: 008-018, 028-035, 037, 039-046, 048, 049, 051-055, 057, 058, 061, 064-067, 069, 074, 077-084, 086, 087, 089-091, 094-098, 100-103, 105, 107, 108, 113, 117, 126, 132-134, 139-141, 144-149, 152-154, 157, 160, 165, 168, 170, 172, 174, 175, 177, 178, 192, 193-207, 214-218, 220, 221, 227-231, 243, 252, 254, 258-263, 265-270, 273, 278-281, 283, 291, 294-299, 306, 312, 315, 316, 324-326, 330, 331, 341, 351, 353, 354, 362-364, 368-370, 379, 384, 388-390, 394-397, 399, 401, 404-406, 410, 417, 419, 420, 422-424, 426-428, 430, 437, 439, 441-443, 445, 454, 458, 460, 464, 468, 469, 471, 474-476, 480-482, 486-498, 503, 507, 508, 512-518, 520-525, 527-529, 533, 536-540, 542, 547, 552, 554, 555, 559, 567, 568, 570, 571, 577, 581, 582-586, 594-597, 599, 602, 607, 608, 614, 617, 619, 621, 622, 625-634, 636-642, 645, 646, 648, 650-656.

47. A method comprising applying a therapeutic that at least partially compensates for an endometriosis related condition to a patient at altered risk of an endometriosis related condition and having at least one endometriosis altered risk genetic marker in said patient.

48. The method of claim 47, wherein said patient is asymptomatic of an endometriosis condition.

49. The method of claim 47, wherein said therapeutic application further defines treating said patient with at least one of a medical device, a pharmaceutical, and a medical device and a pharmaceutical.

50. The method of claim 47, wherein said therapeutic further defines at least one of a protein, a protein fragment, a peptide, an antibody, a nucleic acid, and a mimetic which modulates the function of a target gene.

51. The method of claim 47, wherein said method further includes the step of recording at least one non-genetic endometriosis related clinical factor of said patient.

52. The method of claim 51, wherein said non-genetic endometriosis related clinical factor defines at least one of age at menarche and BMI (Body Mass Index).

53. The method of claim 47, wherein said endometriosis altered risk genetic marker defines the minor allele of at least one SNP selected from the group of SEQ ID No:1 through SEQ ID No:156 and SEQ ID No:158 through SEQ ID No:656.

54. The method of claim 53, wherein said altered risk genetic marker defines at least one of an increased risk endometriosis genetic marker defining the minor allele of at least one SNP selected from the group of SEQ ID NOs: 001-007, 019-027, 036, 038, 047, 050, 056, 059, 060, 062, 063, 068, 070-073, 075, 076, 085, 088, 092, 093, 099, 104, 106, 109, 110-112, 114-116, 118-125, 127-131, 135-137, 138, 142, 143, 150, 151, 155, 156, 158, 159, 161-164, 166, 167, 169, 171, 173, 176, 179-191, 208-213, 219, 222-226, 232, 233, 234-242, 244-251, 245, 253, 255-257, 264, 271, 272, 274-277, 282, 284-290, 292, 293, 300-305, 307-311, 313, 314, 317-323, 327-329, 332-340, 342-350, 352, 355-361, 365-367, 371-378, 380-383, 385-387, 391-393, 398, 400, 402, 403, 407-409, 411-416, 418, 421, 425, 429, 431-436, 438, 440, 444, 446-453, 455-457, 459, 461-463, 465-467, 470, 472, 473, 477-479, 483-485, 499, 500-502, 504-506, 509-511, 519, 526, 530-532, 534, 535, 541, 543-546, 548-551, 553, 556-558, 560-566, 569, 572-576, 578-580, 587-593, 598, 600, 601, 603-606, 609-613, 615, 616, 618, 620, 623, 624, 635, 643, 644, 647, 649, and a decreased risk endometriosis genetic marker defining the minor allele of at least one SNP selected from the group of SEQ ID NOs: 008-018, 028-035, 037, 039-046, 048, 049, 051-055, 057, 058, 061, 064-067, 069, 074, 077-084, 086, 087, 089-091, 094-098, 100-103, 105, 107, 108, 113, 117, 126, 132-134, 139-141, 144-149, 152-154, 157, 160, 165, 168, 170, 172, 174, 175, 177, 178, 192, 193-207, 214-218, 220, 221, 227-231, 243, 252, 254, 258-263, 265-270, 273, 278-281, 283, 291, 294-299, 306, 312, 315, 316, 324-326, 330, 331, 341, 351, 353, 354, 362-364, 368-370, 379, 384, 388-390, 394-397, 399, 401, 404-406, 410, 417, 419, 420, 422-424, 426-428, 430, 437, 439, 441-443, 445, 454, 458, 460, 464, 468, 469, 471, 474-476, 480-482, 486-498, 503, 507, 508, 512-518, 520-525, 527-529, 533, 536-540, 542, 547, 552, 554, 555, 559, 567, 568, 570, 571, 577, 581, 582-586, 594-597, 599, 602, 607, 608, 614, 617, 619, 621, 622, 625-634, 636-642, 645, 646, 648, 650-656.

55. A method comprising administering a pharmaceutical that at least partially compensates for an endometriosis related condition to a patient asymptomatic of an endometriosis condition and at altered risk of an endometriosis related condition and having at least one endometriosis altered risk genetic marker in the DNA of said patient, wherein said endometriosis altered risk genetic marker defines the minor allele of at least one SNP selected from the group of SEQ ID No:1 through SEQ ID No:156 and SEQ ID No:158 through SEQ ID No:656.

56. The method of claim 55, wherein said method further includes the step of obtaining a genetic material sample of a human subject.

57. The method of claim 55, wherein said method further includes the step of recording at least one non-genetic endometriosis related clinical factor of said patient.

58. The method of claim 57, wherein said non-genetic endometriosis related clinical factor defines at least one of age at menarche and BMI (Body Mass Index).

59. The method of claim 55, wherein said altered risk genetic marker defines at least one of an increased risk endometriosis genetic marker defining the minor allele of at least one SNP selected from the group of SEQ ID NOs: 001-007, 019-027, 036, 038, 047, 050, 056, 059, 060, 062, 063, 068, 070-073, 075, 076, 085, 088, 092, 093, 099, 104, 106, 109, 110-112, 114-116, 118-125, 127-131, 135-137, 138, 142, 143, 150, 151, 155, 156, 158, 159, 161-164, 166, 167, 169, 171, 173, 176, 179-191, 208-213, 219, 222-226, 232, 233, 234-242, 244-251, 245, 253, 255-257, 264, 271, 272, 274-277, 282, 284-290, 292, 293, 300-305, 307-311, 313, 314, 317-323, 327-329, 332-340, 342-350, 352, 355-361, 365-367, 371-378, 380-383, 385-387, 391-393, 398, 400, 402, 403, 407-409, 411-416, 418, 421, 425, 429, 431-436, 438, 440, 444, 446-453, 455-457, 459, 461-463, 465-467, 470, 472, 473, 477-479, 483-485, 499, 500-502, 504-506, 509-511, 519, 526, 530-532, 534, 535, 541, 543-546, 548-551, 553, 556-558, 560-566, 569, 572-576, 578-580, 587-593, 598, 600, 601, 603-606, 609-613, 615, 616, 618, 620, 623, 624, 635, 643, 644, 647, 649, and a decreased risk endometriosis genetic marker defining the minor allele of at least one SNP selected from the group of SEQ ID NOs: 008-018, 028-035, 037, 039-046, 048, 049, 051-055, 057, 058, 061, 064-067, 069, 074, 077-084, 086, 087, 089-091, 094-098, 100-103, 105, 107, 108, 113, 117, 126, 132-134, 139-141, 144-149, 152-154, 157, 160, 165, 168, 170, 172, 174, 175, 177, 178, 192, 193-207, 214-218, 220, 221, 227-231, 243, 252, 254, 258-263, 265-270, 273, 278-281, 283, 291, 294-299, 306, 312, 315, 316, 324-326, 330, 331, 341, 351, 353, 354, 362-364, 368-370, 379, 384, 388-390, 394-397, 399, 401, 404-406, 410, 417, 419, 420, 422-424, 426-428, 430, 437, 439, 441-443, 445, 454, 458, 460, 464, 468, 469, 471, 474-476, 480-482, 486-498, 503, 507, 508, 512-518, 520-525, 527-529, 533, 536-540, 542, 547, 552, 554, 555, 559, 567, 568, 570, 571, 577, 581, 582-586, 594-597, 599, 602, 607, 608, 614, 617, 619, 621, 622, 625-634, 636-642, 645, 646, 648, 650-656.


SEQUENCE LISTING (656 sequences)

SEQ ID NO: 1 - DNA, 33 bp, Homo Sapiens
agttatggaaaaggcamctttcaagagggcaaa

SEQ ID NO: 2 - DNA, 33 bp, Homo Sapiens
caagtgtagacccaaartaactgaaaacaggtg

SEQ ID NO: 3 - DNA, 33 bp, Homo Sapiens
aaagagcaaaacagatkaaagaaggaacctggg

SEQ ID NO: 4 - DNA, 33 bp, Homo Sapiens
gaagctccagtccttaratttattccttgctgt

SEQ ID NO: 5 - DNA, 33 bp, Homo Sapiens
attaatgtaaaaaaaayagaccatctcgtgttg

SEQ ID NO: 6 - DNA, 33 bp, Homo Sapiens
gagacaggaaggtgacrgagtggtatcagaccc

SEQ ID NO: 7 - DNA, 33 bp, Homo Sapiens
ctcccaaccttgaacartataaggactcaggtt

SEQ ID NO: 8 - DNA, 33 bp, Homo Sapiens
atacgagcagcagggcraaagaattttccagtt

SEQ ID NO: 9 - DNA, 33 bp, Homo Sapiens
gtgagaacctcagctarggctgtcaattagaac

SEQ ID NO: 10 - DNA, 33 bp, Homo Sapiens
aaatatcccccacacasagaaaagttcaaagac

SEQ ID NO: 11 - DNA, 33 bp, Homo Sapiens
aagactagaaagtccartagcagacttcacact

SEQ ID NO: 12 - DNA, 33 bp, Homo Sapiens
agattcaatcgaatccrcataactcccaaattt

SEQ ID NO: 13 - DNA, 33 bp, Homo Sapiens
agaaggctatttattaygctctttttcttcctc

SEQ ID NO: 14 - DNA, 33 bp, Homo Sapiens
ggactacagtgcataamcaactgattatgaatc

SEQ ID NO: 15 - DNA, 33 bp, Homo Sapiens
tgaagaaaatgaaaaaragatagatggaatttg

SEQ ID NO: 16 - DNA, 33 bp, Homo Sapiens
ggcaatacaacctgatsagaatttatctacctt

SEQ ID NO: 17 - DNA, 33 bp, Homo Sapiens
atacagcaaaaggcaasagcactagaacatgtt

SEQ ID NO: 18 - DNA, 33 bp, Homo Sapiens
tttttcttctcagctarcttccatgtaacatct

SEQ ID NO: 19 - DNA, 33 bp, Homo Sapiens
aatgtgtttccttcaakttccttgtttctatca

SEQ ID NO: 20 - DNA, 33 bp, Homo Sapiens
aattctttaaaatcacrggtccagttgcgttca

SEQ ID NO: 21 - DNA, 33 bp, Homo Sapiens
gtacctttttagtggcrgatcaaacagaaaatt

SEQ ID NO: 22 - DNA, 33 bp, Homo Sapiens
ctgttattttaaatgaragaggctggtaacaac

SEQ ID NO: 23 - DNA, 33 bp, Homo Sapiens
tgtgttacaaaaagcayagcatactataaacac

SEQ ID NO: 24 - DNA, 33 bp, Homo Sapiens
aagcatatataacagargcaacctgaccaaatt

SEQ ID NO: 25 - DNA, 33 bp, Homo Sapiens
gatactgttctctttcraataggatccattttt

SEQ ID NO: 26 - DNA, 33 bp, Homo Sapiens
tttgtacttgtaactaygacattattcggtcaa

SEQ ID NO: 27 - DNA, 33 bp, Homo Sapiens
atttcaacgtttcttayatttcccatggcttac

SEQ ID NO: 28 - DNA, 33 bp, Homo Sapiens
ggcaaaataaacattcyagatcgactgagatgt

SEQ ID NO: 29 - DNA, 33 bp, Homo Sapiens
gcatcttctgttctacraaagaataggctgtca

SEQ ID NO: 30 - DNA, 33 bp, Homo Sapiens
ttttcttgaccactcartataaaaagtcccaat

SEQ ID NO: 31 - DNA, 33 bp, Homo Sapiens
aggaacatgaggcagarctaataactgtcaaca

SEQ ID NO: 32 - DNA, 33 bp, Homo Sapiens
atgttcactgtctccaraagtttccttttgtgg

SEQ ID NO: 33 - DNA, 33 bp, Homo Sapiens
agttgtaaatatttgaytaataaaggacatgta

SEQ ID NO: 34 - DNA, 33 bp, Homo Sapiens
tgagccttctctctgcrcagtgagtctcaaagg

SEQ ID NO: 35 - DNA, 33 bp, Homo Sapiens
tccattggatcattcasgcaacctctctgattt

SEQ ID NO: 36 - DNA, 33 bp, Homo Sapiens
tatactccaggcatggraactacttcacattcc

SEQ ID NO: 37 - DNA, 33 bp, Homo Sapiens
gtgactttagaagatayccaacgtcaccttaat

SEQ ID NO: 38 - DNA, 33 bp, Homo Sapiens
tttaccatgcttccatracaaaggtagtttcta

SEQ ID NO: 39 - DNA, 33 bp, Homo Sapiens
gaagtgagtgatctaarctattgtatagaggta

SEQ ID NO: 40 - DNA, 33 bp, Homo Sapiens
ttaggcaaaaataaaakgtttcaaggattcctg

SEQ ID NO: 41 - DNA, 33 bp, Homo Sapiens
tatgttgataaacataraagccacagtgttaac

SEQ ID NO: 42 - DNA, 33 bp, Homo Sapiens
cctagtacggcacctgsaatataagaaatattc

SEQ ID NO: 43 - DNA, 33 bp, Homo Sapiens
cactgatgctgaatagrcagaatccctccttac

SEQ ID NO: 44 - DNA, 33 bp, Homo Sapiens
atataggaatggccaasaaacttatggaaaaat

SEQ ID NO: 45 - DNA, 33 bp, Homo Sapiens
ccaccaccacaactcayagacccagctgattac

SEQ ID NO: 46 - DNA, 33 bp, Homo Sapiens
aggcatcaggaatggargagcaagatattgagg

SEQ ID NO: 47 - DNA, 33 bp, Homo Sapiens
ctaccatgacaccaccraaggctttgtaccagc

SEQ ID NO: 48 - DNA, 33 bp, Homo Sapiens
aataggtagatttgacrcaacttgggcttccag

SEQ ID NO: 49 - DNA, 33 bp, Homo Sapiens
tttgagttagtgttgamatgagataagactttg

SEQ ID NO: 50 - DNA, 33 bp, Homo Sapiens
accaagctatcctgaargttagagcagagctac

SEQ ID NO: 51 - DNA, 33 bp, Homo Sapiens
tataattaaggtctaaygccatgccacaattga

SEQ ID NO: 52 - DNA, 33 bp, Homo Sapiens
agccggtctccaagacracggcgtgcaggctgg

SEQ ID NO: 53 - DNA, 33 bp, Homo Sapiens
atgatctggttctcaargattaaaaacatgaaa

SEQ ID NO: 54 - DNA, 33 bp, Homo Sapiens
tgtcagtcaaatatgtraagagtgaagaggaag

SEQ ID NO: 55 - DNA, 33 bp, Homo Sapiens
agttccagaagcattgycagtgctaagagtgaa

SEQ ID NO: 56 - DNA, 33 bp, Homo Sapiens
attctactaagagtagraagcattgacaaaaaa

SEQ ID NO: 57 - DNA, 33 bp, Homo Sapiens
attcaagtagataagayaacccacagaacttag

SEQ ID NO: 58 - DNA, 33 bp, Homo Sapiens
gacatccctcagacagragagagaataagcagc

SEQ ID NO: 59 - DNA, 33 bp, Homo Sapiens
gcaaaattgaggcagartcataacagacagtat

SEQ ID NO: 60 - DNA, 33 bp, Homo Sapiens
atgaaggacttacgcawctattttggagattca

SEQ ID NO: 61 - DNA, 33 bp, Homo Sapiens
atactctgtgtgatgcratatacgaaggatata

SEQ ID NO: 62 - DNA, 33 bp, Homo Sapiens
tgttcactgttttgaayaaagcactgatctaaa

SEQ ID NO: 63 - DNA, 33 bp, Homo Sapiens
gtttttatttactactsacaaaaaggtactatg

SEQ ID NO: 64 - DNA, 33 bp, Homo Sapiens
atctcccctctcattcratcagatagataaaga

SEQ ID NO: 65 - DNA, 33 bp, Homo Sapiens
gtgaaatgtgaacgcgyaccattatcttgtgac

SEQ ID NO: 66 - DNA, 33 bp, Homo Sapiens
atatgcaaacttatcartcagaactcacccctc

SEQ ID NO: 67 - DNA, 33 bp, Homo Sapiens
actgatggaggaaagartagtccttgaagtaga

SEQ ID NO: 68 - DNA, 33 bp, Homo Sapiens
ttttaaaaagaaagaartgcaaagattgaacag

SEQ ID NO: 69 - DNA, 33 bp, Homo Sapiens
atttttaaaatgttatsaaccagggtaaaagat

SEQ ID NO: 70 - DNA, 33 bp, Homo Sapiens
caaagatcttacaaagscttactgctgttcttc

SEQ ID NO: 71 - DNA, 33 bp, Homo Sapiens
ttttcaatgcgatgtcraagtagggcaggctct

SEQ ID NO: 72 - DNA, 33 bp, Homo Sapiens
gaacacagcattcctcmaaattcttagtactct

SEQ ID NO: 73 - DNA, 33 bp, Homo Sapiens
ctgccttctcagagttraacctgacataattta

SEQ ID NO: 74 - DNA, 33 bp, Homo Sapiens
aggaataagtttaaccrgatgcttgaagttaac

SEQ ID NO: 75 - DNA, 33 bp, Homo Sapiens
gtctttttactgctacyaaaactacccaagtgt

SEQ ID NO: 76 - DNA, 33 bp, Homo Sapiens
ctccactaggtgtttaygggttgactgggaaaa

SEQ ID NO: 77 - DNA, 33 bp, Homo Sapiens
cttattaagctgattaygcaataggcttaacat

SEQ ID NO: 78 - DNA, 33 bp, Homo Sapiens
ttccaaattctccaacmgaagagtctcctattc

SEQ ID NO: 79 - DNA, 33 bp, Homo Sapiens
cactgaagctcagaaakaaacttatgagacagc

SEQ ID NO: 80 - DNA, 33 bp, Homo Sapiens
catgagattattagagraaaatgtgcttggccc

SEQ ID NO: 81 - DNA, 33 bp, Homo Sapiens
ggatcctctcttcacartcaaatttgcctaggt

SEQ ID NO: 82 - DNA, 33 bp, Homo Sapiens
agccttcctgtagaacwcgagtaggtaatcgaa

SEQ ID NO: 83 - DNA, 33 bp, Homo Sapiens
atagatgggtcgatggyaggcattcatcttgga

SEQ ID NO: 84 - DNA, 33 bp, Homo Sapiens
cagcccagagtgagaaytgcaaagaaggaaaaa

SEQ ID NO: 85 - DNA, 33 bp, Homo Sapiens
ggctggcttctgtcacrggagaacctgtctaaa

SEQ ID NO: 86 - DNA, 33 bp, Homo Sapiens
gatgatctttctgtccrcaaggttcttatttcc

SEQ ID NO: 87 - DNA, 33 bp, Homo Sapiens
aatctttgcctctaaayactttcctctttaatt

SEQ ID NO: 88 - DNA, 33 bp, Homo Sapiens
cggtttggaccgaacayggcaacctcatcacct

SEQ ID NO: 89 - DNA, 33 bp, Homo Sapiens
atttggtgaaagtccaygcaaccattttcacaa

SEQ ID NO: 90 - DNA, 33 bp, Homo Sapiens
gccaaaggcatggaacygattctagccccatca

SEQ ID NO: 91 - DNA, 33 bp, Homo Sapiens
tttacatggctaaaagwaaactttccttttcac

SEQ ID NO: 92 - DNA, 33 bp, Homo Sapiens
tctcactgtgggtcacraacaaaaactttgata

SEQ ID NO: 93 - DNA, 33 bp, Homo Sapiens
aatgggatttcattatsagtatccatcagtcat

SEQ ID NO: 94 - DNA, 33 bp, Homo Sapiens
acaccttcagaacaccrcgagtctcctcataga

SEQ ID NO: 95 - DNA, 33 bp, Homo Sapiens
tgcctggttggccataygcgtccaatttttcac

SEQ ID NO: 96 - DNA, 33 bp, Homo Sapiens
cataatctgggaatgcracccggaattattaag

SEQ ID NO: 97 - DNA, 33 bp, Homo Sapiens
tcagaagcgaagaagargtaatgtccaggatgg

SEQ ID NO: 98 - DNA, 33 bp, Homo Sapiens
tatccttttcgcatacwcataatttcaagaatc

SEQ ID NO: 99 - DNA, 33 bp, Homo Sapiens
tgcatagctgtaataasagttgaggaggaatct

SEQ ID NO: 100 - DNA, 33 bp, Homo Sapiens
ttttatcagacgcagaragagagagaactggga

SEQ ID NO: 101 - DNA, 33 bp, Homo Sapiens
ctagatttaatagctakcagggatttcgagttg

SEQ ID NO: 102 - DNA, 33 bp, Homo Sapiens
agtgacaagtagacccrgagataaacaactcga

SEQ ID NO: 103 - DNA, 33 bp, Homo Sapiens
tctggaaatgctagacragtagtatagcaactc

SEQ ID NO: 104 - DNA, 33 bp, Homo Sapiens
aaagtgagacagaagasaattggtattttctct

SEQ ID NO: 105 - DNA, 33 bp, Homo Sapiens
caataaaattttattcrcaataagtgctataga

SEQ ID NO: 106 - DNA, 33 bp, Homo Sapiens
actttgatacttaaccygctacccggataaaac

SEQ ID NO: 107 - DNA, 33 bp, Homo Sapiens
gggaaatcagatcggaraactagaggtttgcca

SEQ ID NO: 108 - DNA, 33 bp, Homo Sapiens
gtgtactgggcaatcaygttttccttttctaat

SEQ ID NO: 109 - DNA, 33 bp, Homo Sapiens
gtaacacatcatcagaycttgttattttgaaga

SEQ ID NO: 110 - DNA, 33 bp, Homo Sapiens
gcacatgtctgaaaaamtattgaggttgttaag

SEQ ID NO: 111 - DNA, 33 bp, Homo Sapiens
attttacttctactgartagcagtcatgattta

SEQ ID NO: 112 - DNA, 33 bp, Homo Sapiens
ttctctgtctagccaarctgctgttagaagaat

SEQ ID NO: 113 - DNA, 33 bp, Homo Sapiens
agactgttgagtctgcrcgtccttgttcctgcg

SEQ ID NO: 114 - DNA, 33 bp, Homo Sapiens
tttattcatggactaamatccttgtctgacttt

SEQ ID NO: 115 - DNA, 33 bp, Homo Sapiens
tgtctccagtgtggagratatacttcaacaatt

SEQ ID NO: 116 - DNA, 33 bp, Homo Sapiens
cttatcagattaatgcraccctgatagatcccc

SEQ ID NO: 117 - DNA, 33 bp, Homo Sapiens
aggcttttcctgcttcmacgtgggggtactgct

SEQ ID NO: 118 - DNA, 33 bp, Homo Sapiens
atcctgtggataaaacygtataaatgtgagaga

SEQ ID NO: 119 - DNA, 33 bp, Homo Sapiens
gtcaaacgacaagttaygtcatccactggaaca

SEQ ID NO: 120 - DNA, 33 bp, Homo Sapiens
taggtaagctagaatarcctgagccaaaaaata

SEQ ID NO: 121 - DNA, 33 bp, Homo Sapiens
aatgaagattcaggacracgaagcagtggtaac

SEQ ID NO: 122 - DNA, 33 bp, Homo Sapiens
gagaaagagagagaaayagcgctgctcagggga

SEQ ID NO: 123 - DNA, 33 bp, Homo Sapiens
taaattataccacgcaytgagccaaagccgctg

SEQ ID NO: 124 - DNA, 33 bp, Homo Sapiens
catctgatttcccaaayggacactcctagcaga

SEQ ID NO: 125 - DNA, 33 bp, Homo Sapiens
tggctaagtattttagyagcaatagtcagcaca

SEQ ID NO: 126 - DNA, 33 bp, Homo Sapiens
tggctgaaaactagaayggtgtcattgtattgc

SEQ ID NO: 127 - DNA, 33 bp, Homo Sapiens
gattataagtttctgayattgtatctgagaaga

SEQ ID NO: 128 - DNA, 33 bp, Homo Sapiens
tcagtcatctatatccraaatgtatctgagtta

SEQ ID NO: 129 - DNA, 33 bp, Homo Sapiens
cttatatttaagataaktagcctgccacaatta

SEQ ID NO: 130 - DNA, 33 bp, Homo Sapiens
agaatgtagaggcagasgaaatacagtgaggga

SEQ ID NO: 131 - DNA, 33 bp, Homo Sapiens
gtaacttccatgctctyacttttttatacctct

SEQ ID NO: 132 - DNA, 33 bp, Homo Sapiens
accttcctgagtcatasaaagtcacttgacctc

SEQ ID NO: 133 - DNA, 33 bp, Homo Sapiens
attaaaattttcccgartagcttctgcttgact

SEQ ID NO: 134 - DNA, 33 bp, Homo Sapiens
ggacccacttcccagcwctgttgcactggaaat

SEQ ID NO: 135 - DNA, 33 bp, Homo Sapiens
attctatatgtgtttayggggtgtaatctctgt

SEQ ID NO: 136 - DNA, 33 bp, Homo Sapiens
caagcttttccttcaaygaacctttgtaggcat

SEQ ID NO: 137 - DNA, 33 bp, Homo Sapiens
agaaaggtacacgccayaaatgccgacattcaa

SEQ ID NO: 138 - DNA, 33 bp, Homo Sapiens
atactaaagggagtcargaggacaactcagcaa

SEQ ID NO: 139 - DNA, 33 bp, Homo Sapiens
tttttgctcacagaacraggtagctgcatgcag

SEQ ID NO: 140 - DNA, 33 bp, Homo Sapiens
tttttaaggagataaamaagagttgagtctggg

SEQ ID NO: 141 - DNA, 33 bp, Homo Sapiens
tcattcagatttaacckcgtcaaagagtagttc

SEQ ID NO: 142 - DNA, 33 bp, Homo Sapiens
aacaccagggaatgaaratttcgcttagtgaag

SEQ ID NO: 143 - DNA, 33 bp, Homo Sapiens
tacagtgtgtacccaaygattgcttagtattgt

SEQ ID NO: 144 - DNA, 33 bp, Homo Sapiens
tacttcaagggttccartgagattttctgagct

SEQ ID NO: 145 - DNA, 33 bp, Homo Sapiens
aataatgtatgctacarcacaaagcatttatat

SEQ ID NO: 146 - DNA, 33 bp, Homo Sapiens
cagagagaacactgtaraaggattctgagctgg

SEQ ID NO: 147 - DNA, 33 bp, Homo Sapiens
cctcccaaagtgctggwaatccgggaatgaacc

SEQ ID NO: 148 - DNA, 33 bp, Homo Sapiens
ccaagagaggtcagaakaattcaatctgatgtg

SEQ ID NO: 149 - DNA, 33 bp, Homo Sapiens
ttgatcaaagctgaagkattagggcaagtcact

SEQ ID NO: 150 - DNA, 33 bp, Homo Sapiens
gcccgtagggagtatcrcttgacggagagtgag

SEQ ID NO: 151 - DNA, 33 bp, Homo Sapiens
cacattcttttctctayaccaggggtggcaaac

SEQ ID NO: 152 - DNA, 33 bp, Homo Sapiens
cagtttcagggtagccwacaacaaagactggct

SEQ ID NO: 153 - DNA, 33 bp, Homo Sapiens
gtcttactgctttcackccaattgtcattccag

SEQ ID NO: 154 - DNA, 33 bp, Homo Sapiens
cccttgggcagccagaraaaattcacaataagc

SEQ ID NO: 155 - DNA, 33 bp, Homo Sapiens
tggtgcctcacgcccaygaaagaaggctgccac

SEQ ID NO: 156 - DNA, 33 bp, Homo Sapiens
acggcattgcttgactractttaataaaactgt

SEQ ID NO: 157 - DNA, 33 bp, Homo Sapiens
gagttgtgcaatcatcrctgtaacctaatttta

SEQ ID NO: 158 - DNA, 33 bp, Homo Sapiens
ccaacatatgatagaakgggaaaaagcaacaaa

SEQ ID NO: 159 - DNA, 33 bp, Homo Sapiens
tttctccaatttccaayaatagagtctccaaag

SEQ ID NO: 160 - DNA, 33 bp, Homo Sapiens
aattagcagaatgaaargaatgaaaagatgctg

SEQ ID NO: 161 - DNA, 33 bp, Homo Sapiens
tattgaatctgttatcratgccatgggacaaat

SEQ ID NO: 162 - DNA, 33 bp, Homo Sapiens
atgaatgataacctgayagaagacactgctttg

SEQ ID NO: 163 - DNA, 33 bp, Homo Sapiens
taatttctattttgagycaaaaggtgttattat

SEQ ID NO: 164 - DNA, 33 bp, Homo Sapiens
ctctcttccaggattgyagctcttatccaagtc

SEQ ID NO: 165 - DNA, 33 bp, Homo Sapiens
aaagccagagttagaargtaaggacactacccc

SEQ ID NO: 166 - DNA, 33 bp, Homo Sapiens
aggcaatgacttccacsaggaatacacgtcatt

SEQ ID NO: 167 - DNA, 33 bp, Homo Sapiens
cgcacagctgaaatgaygggtttcactgcaggg

SEQ ID NO: 168 - DNA, 33 bp, Homo Sapiens
tcccatgcgtgttttargatagactagttcagt

SEQ ID NO: 169 - DNA, 33 bp, Homo Sapiens
acctaagctgaagaacygggaagtgtgataaag

SEQ ID NO: 170 - DNA, 33 bp, Homo Sapiens
tggcatcatggaaaagyaccagcttggttttgg

SEQ ID NO: 171 - DNA, 33 bp, Homo Sapiens
tttggaaatatggtcaygctagatgtaactagt

SEQ ID NO: 172 - DNA, 33 bp, Homo Sapiens
catccaaaagcagtgasatgaccattcaagagc

SEQ ID NO: 173 - DNA, 33 bp, Homo Sapiens
cagttctcaactagcascactcaagtttaataa

SEQ ID NO: 174 - DNA, 33 bp, Homo Sapiens
ccatgtactaaaattayacaagaaatgagctgg

SEQ ID NO: 175 - DNA, 33 bp, Homo Sapiens
gtcccaggtctagcccratagtgcggaagtagc

SEQ ID NO: 176 - DNA, 33 bp, Homo Sapiens
atcaccctgcctgaaayacactcaagggtaccc

SEQ ID NO: 177 - DNA, 33 bp, Homo Sapiens
ctctaaacattaaaaamcatgagtttacctcaa

SEQ ID NO: 178 - DNA, 33 bp, Homo Sapiens
aggtttagttattgtcygaatgagatagtttcc

SEQ ID NO: 179 - DNA, 33 bp, Homo Sapiens
cttcctctttcctctcraagtagtagggggcgc

SEQ ID NO: 180 - DNA, 33 bp, Homo Sapiens
taacatgatatatcacyaacatctgaatttgat

SEQ ID NO: 181 - DNA, 33 bp, Homo Sapiens
tatcccaagcacatagwcaaatgccaggtgcat

SEQ ID NO: 182 - DNA, 33 bp, Homo Sapiens
ggcaaatcatagtagaraggaagcaactccaca

SEQ ID NO: 183 - DNA, 33 bp, Homo Sapiens
gaacagctatgccacartaggtcacttactgga

SEQ ID NO: 184 - DNA, 33 bp, Homo Sapiens
agaatgcgaaacagtcsagctgctatggaaaac

SEQ ID NO: 185 - DNA, 33 bp, Homo Sapiens
gttagagatttgacaaygaactagaagataagg

SEQ ID NO: 186 - DNA, 33 bp, Homo Sapiens
ctgctctagccagaccrctctttgcaaacactt

SEQ ID NO: 187 - DNA, 33 bp, Homo Sapiens
gtccctggagaaacacrattagcgggcgaagtt

SEQ ID NO: 188 - DNA, 33 bp, Homo Sapiens
cctgcaaagtcatagaygtgtttatttttcctt

SEQ ID NO: 189 - DNA, 33 bp, Homo Sapiens
aagccagaagtattctyacctagtccagtacta

SEQ ID NO: 190 - DNA, 33 bp, Homo Sapiens
gctgatccctgatagcrcagcttcacagagcac

SEQ ID NO: 191 - DNA, 33 bp, Homo Sapiens
attttgtacggttttcmcttacatcctttctat

SEQ ID NO: 192 - DNA, 33 bp, Homo Sapiens
ccaaggtgatcagaaamcaagtggacaccacca

SEQ ID NO: 193 - DNA, 33 bp, Homo Sapiens
aggctagtttcatccartgaggaatcttggggc

SEQ ID NO: 194 - DNA, 33 bp, Homo Sapiens
gcggcagtaaaaatggrcaactatagctatgca

SEQ ID NO: 195 - DNA, 33 bp, Homo Sapiens
gccggtgaagtttacawgtatttattacagatt

SEQ ID NO: 196 - DNA, 33 bp, Homo Sapiens
gcaatagccatggtcaytgaaaacaaggctctg

SEQ ID NO: 197 - DNA, 33 bp, Homo Sapiens
ctcaaactcctgacctsagacaatgcacctgct

SEQ ID NO: 198 - DNA, 33 bp, Homo Sapiens
aagattgggacaggtargtttttttaatattca

SEQ ID NO: 199 - DNA, 33 bp, Homo Sapiens
ctgtactccaatatcasaatttacaggtaagta

SEQ ID NO: 200 - DNA, 33 bp, Homo Sapiens
tgtgctctcaagggtaygctacaggctgagtgc

SEQ ID NO: 201 - DNA, 33 bp, Homo Sapiens
tcacccagagttacaaycaggccaggccaatca

SEQ ID NO: 202 - DNA, 33 bp, Homo Sapiens
tggggacttgggaaaaraaatcggccagagcct

SEQ ID NO: 203 - DNA, 33 bp, Homo Sapiens
cttatgatgaatagaasagtcacacttgcttgg

SEQ ID NO: 204 - DNA, 33 bp, Homo Sapiens
aagagaaagatcacctracttaagagctgggcc

SEQ ID NO: 205 - DNA, 33 bp, Homo Sapiens
acaacaaaaaaagcccraagagccaatgcaatc

SEQ ID NO: 206 - DNA, 33 bp, Homo Sapiens
ccacagtctgcgccccragttctccttggccaa

SEQ ID NO: 207 - DNA, 33 bp, Homo Sapiens
gtgagagtgccgggtcygacatcctgagaaaag

SEQ ID NO: 208 - DNA, 33 bp, Homo Sapiens
agccagaaggctgtcawcagaaggaccccacag

SEQ ID NO: 209 - DNA, 33 bp, Homo Sapiens
gagctatcctaacacartaagagaggaaaagca

SEQ ID NO: 210 - DNA, 33 bp, Homo Sapiens
gctagattcctcctcakgtctactgatttggaa

SEQ ID NO: 211 - DNA, 33 bp, Homo Sapiens
tatgagaggtcctcgayaaatatttgtggaaag

SEQ ID NO: 212 - DNA, 33 bp, Homo Sapiens
tttgtgtaaggggcacsagatttccactgaata

SEQ ID NO: 213 - DNA, 33 bp, Homo Sapiens
gcctttctgcctgtacractccagtgtgtacct

SEQ ID NO: 214 - DNA, 33 bp, Homo Sapiens
ctgatgctgccagttckgaaactacattttgag

SEQ ID NO: 215 - DNA, 33 bp, Homo Sapiens
caacagagtgaaggacyggctacataacttgta

SEQ ID NO: 216 - DNA, 33 bp, Homo Sapiens
actctcattatgacgargagtaaatttccatca

SEQ ID NO: 217 - DNA, 33 bp, Homo Sapiens
tttgagagtgttcatgrcagtattgcttagaac

SEQ ID NO: 218 - DNA, 33 bp, Homo Sapiens
tctgctacaaaatcgcyaaaggtggggacactg

SEQ ID NO: 219 - DNA, 33 bp, Homo Sapiens
actaaagagcctgaaawtgtaataataccatat

SEQ ID NO: 220 - DNA, 33 bp, Homo Sapiens
gtaccacctgttgatayagtgaagtgcctgggc

SEQ ID NO: 221 - DNA, 33 bp, Homo Sapiens
ggcgtggcattccttcmaacacctttagtggaa

SEQ ID NO: 222 - DNA, 33 bp, Homo Sapiens
ctcacagctccactccratcctggagtcatcgc

SEQ ID NO: 223 - DNA, 33 bp, Homo Sapiens
acagtaaacagtattasaaaggcaagagagttt

SEQ ID NO: 224 - DNA, 33 bp, Homo Sapiens
tttgggaaactctaccrgatctgttgcaaacta

SEQ ID NO: 225 - DNA, 33 bp, Homo Sapiens
tttaaatgtttcttaaygctccacaaggcagat

SEQ ID NO: 226 - DNA, 33 bp, Homo Sapiens
ctttttctcaaaaataraggaagcaagacatca

SEQ ID NO: 227 - DNA, 33 bp, Homo Sapiens
caggtaagtcttcatamccatcctacacaccct

SEQ ID NO: 228 - DNA, 33 bp, Homo Sapiens
ttggcaaaaaggcctasagagtgatacacttga

SEQ ID NO: 229 - DNA, 33 bp, Homo Sapiens
taaccagctaattgaawtgaagctcctgaaaag

SEQ ID NO: 230 - DNA, 33 bp, Homo Sapiens
tttgcagtttagcacartaatacaaactttgta

SEQ ID NO: 231 - DNA, 33 bp, Homo Sapiens
agccctgagatcttatsaagaatctgctgataa

SEQ ID NO: 232 - DNA, 33 bp, Homo Sapiens
tagagacactgcaaaaytggcacaatcaggcta

SEQ ID NO: 233 - DNA, 33 bp, Homo Sapiens
tatcaaggaatatccayaagcatcaataccatc

SEQ ID NO: 234 - DNA, 33 bp, Homo Sapiens
ggaaagaacgctggcaytgagctctttcccaaa

SEQ ID NO: 235 - DNA, 33 bp, Homo Sapiens
agagtggtttgcagtcratactcagtggtttgg

SEQ ID NO: 236 - DNA, 33 bp, Homo Sapiens
agaggagaagtaggtgmacgggggctcagctga

SEQ ID NO: 237 - DNA, 33 bp, Homo Sapiens
tgtggatgaaagctgakccaactgtcgccacag

SEQ ID NO: 238 - DNA, 33 bp, Homo Sapiens
aataaaaaacatgatarccactatttagagaaa

SEQ ID NO: 239 - DNA, 33 bp, Homo Sapiens
gctgaacactctttccragaaggccaagtttcc

SEQ ID NO: 240 - DNA, 33 bp, Homo Sapiens
catggactgtgttggawaacaagccttaggtcc

SEQ ID NO: 241 - DNA, 33 bp, Homo Sapiens
agacttggaagaaggcygagtgggtaacttgaa

SEQ ID NO: 242 - DNA, 33 bp, Homo Sapiens
ggcctgagaaaaatcckgcttagagaaattaat

SEQ ID NO: 243 - DNA, 33 bp, Homo Sapiens
aaattacagaaaacacraactaatttaaagcaa

SEQ ID NO: 244 - DNA, 33 bp, Homo Sapiens
acaaccttgggaggtaraaacagcaaatgagac

SEQ ID NO: 245 - DNA, 33 bp, Homo Sapiens
tatcagggcatttcaasgctgacattagctgtg

SEQ ID NO: 246 - DNA, 33 bp, Homo Sapiens
aatcactcacctgtcawcgcatcatggcaatat

SEQ ID NO: 247 - DNA, 33 bp, Homo Sapiens
acacattcattaggaakggactatagcatagaa

SEQ ID NO: 248 - DNA, 33 bp, Homo Sapiens
ccactttcctattaagkatacaagctgaaagtt

SEQ ID NO: 249 - DNA, 33 bp, Homo Sapiens
aagatttggaaatgaarccaggcaggaggagag

SEQ ID NO: 250 - DNA, 33 bp, Homo Sapiens
caacttgttagtcagcrccacaagggcatttaa

SEQ ID NO: 251 - DNA, 33 bp, Homo Sapiens
cctttcccttagaccaygaaacatctcataggc

SEQ ID NO: 252 - DNA, 33 bp, Homo Sapiens
ttacagccaaagttgaraacattacactagaat

SEQ ID NO: 253 - DNA, 33 bp, Homo Sapiens
ttcactttcaacagacraatatcttctatgcca

SEQ ID NO: 254 - DNA, 33 bp, Homo Sapiens
gtttacctatctggaakgttctagcagagtctt

SEQ ID NO: 255 - DNA, 33 bp, Homo Sapiens
ttaaaaaaaaaagatcygagcaggccaggcgta

SEQ ID NO: 256 - DNA, 33 bp, Homo Sapiens
attcttccaacttaaamcctggagtcttgtttt

SEQ ID NO: 257 - DNA, 33 bp, Homo Sapiens
gacttgtgtaaagaaakctggattttatctgag

SEQ ID NO: 258 - DNA, 33 bp, Homo Sapiens
atgtattgagagcctaycattttctagaaccca

SEQ ID NO: 259 - DNA, 33 bp, Homo Sapiens
tggcattattccagacyagtttgctaacattaa

SEQ ID NO: 260 - DNA, 33 bp, Homo Sapiens
tctaatgtctcttagcwacctatgcaatctaaa

SEQ ID NO: 261 - DNA, 33 bp, Homo Sapiens
cattttcagattgcacscacatacccagatgac

SEQ ID NO: 262 - DNA, 33 bp, Homo Sapiens
catttcctcttcctcasaaataaggtaaacttt

SEQ ID NO: 263 - DNA, 33 bp, Homo Sapiens
ttagcaccaaaggttamctaatttaagcagctc

SEQ ID NO: 264 - DNA, 33 bp, Homo Sapiens
tatggaaccaaaaaacrgtctatatagccaaga

SEQ ID NO: 265 - DNA, 33 bp, Homo Sapiens
gtggcaggtacgtagtwaaaatcttctggccaa

SEQ ID NO: 266 - DNA, 33 bp, Homo Sapiens
aatattggctccatgarggtaaggaatgtctgt

SEQ ID NO: 267 - DNA, 33 bp, Homo Sapiens
ggtatattgcaggacaraaacttaggggaggaa

SEQ ID NO: 268 - DNA, 33 bp, Homo Sapiens
caacatcatcatttcakaagtctatttgggttt

SEQ ID NO: 269 - DNA, 33 bp, Homo Sapiens
gatgtggcctgtctagratttctgctgaatact

SEQ ID NO: 270 - DNA, 33 bp, Homo Sapiens
ttctggagttgatacascaagtttgaggctccc

SEQ ID NO: 271 - DNA, 33 bp, Homo Sapiens
ttttgtctttctcagcraacatgtacccatgac

SEQ ID NO: 272 - DNA, 33 bp, Homo Sapiens
agtgcctggtaaacatraagtaccttatattag

SEQ ID NO: 273 - DNA, 33 bp, Homo Sapiens
gttgtttgggggtggaytaattagagtaaagct

SEQ ID NO: 274 - DNA, 33 bp, Homo Sapiens
cactcaagttccagcasctttttctctattctg

SEQ ID NO: 275 - DNA, 33 bp, Homo Sapiens
acagcggtaaaggtaayggcatccacctgcgaa

SEQ ID NO: 276 - DNA, 33 bp, Homo Sapiens
ctctcactggtgaaaayggtaaactgggaaata

SEQ ID NO: 277 - DNA, 33 bp, Homo Sapiens
agacaatgagtccagaygcgatagctcacacct

SEQ ID NO: 278 - DNA, 33 bp, Homo Sapiens
ctcattccacactttamggtcatattaaccaag

SEQ ID NO: 279 - DNA, 33 bp, Homo Sapiens
agcagattgcactaaaygtattttattcctgca

SEQ ID NO: 280 - DNA, 33 bp, Homo Sapiens
gcctgctttagtgctcraggtcctgacctgctg

SEQ ID NO: 281 - DNA, 33 bp, Homo Sapiens
gtgtgatgagttctcakggcgttagttctacaa

SEQ ID NO: 282 - DNA, 33 bp, Homo Sapiens
tcagaagccaaagagaraaaggggtcatcattg

SEQ ID NO: 283 - DNA, 33 bp, Homo Sapiens
aaagattctgaactaaratacaaaagaatgtgt

SEQ ID NO: 284 - DNA, 33 bp, Homo Sapiens
gtaggagtagactctayggaacgagaactcagc

SEQ ID NO: 285 - DNA, 33 bp, Homo Sapiens
gtcccctaaccagtaartggagagctacaggaa

SEQ ID NO: 286 - DNA, 33 bp, Homo Sapiens
atcctttttgccatatwacagaacatattcaca

SEQ ID NO: 287 - DNA, 33 bp, Homo Sapiens
atggaatgtctgtacaygtatttttcacttctc

SEQ ID NO: 288 - DNA, 33 bp, Homo Sapiens
tatgtgggggcaccaaraaggcaccgtgaaaat

SEQ ID NO: 289 - DNA, 33 bp, Homo Sapiens
ctgcatgctaaaagtaygtgtacaaattgacaa

SEQ ID NO: 290 - DNA, 33 bp, Homo Sapiens
gctaatgttctaatggkaaacagacattcgaca

SEQ ID NO: 291 - DNA, 33 bp, Homo Sapiens
tggtgcaagttctctgmatggtgatcttcattt

SEQ ID NO: 292 - DNA, 33 bp, Homo Sapiens
ttatcccaaagtgaacmctcatgtcaccagcac

SEQ ID NO: 293 - DNA, 33 bp, Homo Sapiens
gttttttaagatctgcycatgttactgtgtatg

SEQ ID NO: 294 - DNA, 33 bp, Homo Sapiens
gtgatggtatagctatraagcagtgtgagaagt

SEQ ID NO: 295 - DNA, 33 bp, Homo Sapiens
atgcttatcaaagaacwgtcactttatttttcc

SEQ ID NO: 296 - DNA, 33 bp, Homo Sapiens
tggcttccaaagttcamattaaaggcactgtcg

SEQ ID NO: 297 - DNA, 33 bp, Homo Sapiens
agatgtgacaacaaccrcaggagatgggtactt

SEQ ID NO: 298 - DNA, 33 bp, Homo Sapiens
ctgaaaagaaggctaamgtcaaggaaccaagtg

SEQ ID NO: 299 - DNA, 33 bp, Homo Sapiens
aaagtctcatttcttgyactacataaagatagt

SEQ ID NO: 300 - DNA, 33 bp, Homo Sapiens
gaaaagcaggtgaatgkatagagagatgataga

SEQ ID NO: 301 - DNA, 33 bp, Homo Sapiens
ctgagaagactccaggraaaaactatgatatat

SEQ ID NO: 302 - DNA, 33 bp, Homo Sapiens
ttcttcataatctagtraagtcaggaagttcac

SEQ ID NO: 303 - DNA, 33 bp, Homo Sapiens
aactcagcctccatacwcaagtgaggcagaaaa

SEQ ID NO: 304 - DNA, 33 bp, Homo Sapiens
ggaccaaattccctaasgtttgtgaagtaaaat

SEQ ID NO: 305 - DNA, 33 bp, Homo Sapiens
atatgttatttaacccrgatcaccaagttgaat

SEQ ID NO: 306 - DNA, 33 bp, Homo Sapiens
gtttaggctcatgataygaatgtcttcagaaat

SEQ ID NO: 307 - DNA, 33 bp, Homo Sapiens
catagcacttggcttayattgaacctcagaaaa

SEQ ID NO: 308 - DNA, 33 bp, Homo Sapiens
agagaagacatctagamctgtgctggccagcac

SEQ ID NO: 309 - DNA, 33 bp, Homo Sapiens
gttttttcaatgcaaasaataccagtgcctacc

SEQ ID NO: 310 - DNA, 33 bp, Homo Sapiens
gtcacttgataaggaakattgttattagaattt

SEQ ID NO: 311 - DNA, 33 bp, Homo Sapiens
atttggcaaggactgarctagatatcagtgata

SEQ ID NO: 312 - DNA, 33 bp, Homo Sapiens
attaagttggctataaratacaacgattggcaa

SEQ ID NO: 313 - DNA, 33 bp, Homo Sapiens
gccatgtggccttgcayggcatgaggtgtcctc

SEQ ID NO: 314 - DNA, 33 bp, Homo Sapiens
gcaagacagacagctcwctctcagggcacgtag

SEQ ID NO: 315 - DNA, 33 bp, Homo Sapiens
tgcataagtgcaaatcrcagccctggctctgtc

SEQ ID NO: 316 - DNA, 33 bp, Homo Sapiens
ctttatcacaaaagtasagctgaacggattgtg

SEQ ID NO: 317 - DNA, 33 bp, Homo Sapiens
gtgctacaaactttaaytggattgcaaaaaatg

SEQ ID NO: 318 - DNA, 33 bp, Homo Sapiens
tgaattgtagcaggaastagagttgctcactgc

SEQ ID NO: 319 - DNA, 33 bp, Homo Sapiens
gcagtgttctcctaaaytgctcccccttaccac

SEQ ID NO: 320 - DNA, 33 bp, Homo Sapiens
agttcaaaaaaatgccrcagaatgtacttcatc

SEQ ID NO: 321 - DNA, 33 bp, Homo Sapiens
catcttcatgaatttcwaatgagggagagagag

SEQ ID NO: 322 - DNA, 33 bp, Homo Sapiens
tccaagaatcaaccgcyagcagagtaatccgtt

SEQ ID NO: 323 - DNA, 33 bp, Homo Sapiens
tgaaccgttgttcaagyacaattaaggcaaaaa

SEQ ID NO: 324 - DNA, 33 bp, Homo Sapiens
gagtactacagatggcmatgacgccagcttctt

SEQ ID NO: 325 - DNA, 33 bp, Homo Sapiens
tccttcccaaggaagaygcaggttagacaaaca

SEQ ID NO: 326 - DNA, 33 bp, Homo Sapiens
tggtagagctggcacaygatcccaggtctcttc

SEQ ID NO: 327 - DNA, 33 bp, Homo Sapiens
ctcaagggaggacaatragcaagaaccaaaagt

SEQ ID NO: 328 - DNA, 33 bp, Homo Sapiens
agcagggtgaggcagcragtatactgtcatcag

SEQ ID NO: 329 - DNA, 33 bp, Homo Sapiens
gatttctgatcttagascagacaggcgggaggt

SEQ ID NO: 330 - DNA, 33 bp, Homo Sapiens
ttaggaacatatgcccracatgtttttgcttct

SEQ ID NO: 331 - DNA, 33 bp, Homo Sapiens
gcagtgcttcaaacccracacagataatttcta

SEQ ID NO: 332 - DNA, 33 bp, Homo Sapiens
aaacctaaaacaaacamaatctgaatcattttg

SEQ ID NO: 333 - DNA, 33 bp, Homo Sapiens
taagtgatagattataygttgttgctcaaagag

SEQ ID NO: 334 - DNA, 33 bp, Homo Sapiens
attccccctctacacamatgatgaaagtgaggt

SEQ ID NO: 335 - DNA, 33 bp, Homo Sapiens
tactatccacctaggckatacagtatggtctat

SEQ ID NO: 336 - DNA, 33 bp, Homo Sapiens
tcttacaatagagatartaattcctggagttgt

SEQ ID NO: 337 - DNA, 33 bp, Homo Sapiens
ttatctttatctctgaycaacactcaatacttt

SEQ ID NO: 338 - DNA, 33 bp, Homo Sapiens
aatccaagcaccgttayattaattgttttaaca

SEQ ID NO: 339 - DNA, 33 bp, Homo Sapiens
catttccaagaagcaaygcaaagtaagttttcc

SEQ ID NO: 340 - DNA, 33 bp, Homo Sapiens
acagaataagaagtccrcaaatagattcacaca

SEQ ID NO: 341 - DNA, 33 bp, Homo Sapiens
ttaagagtagaagtcartactcaatggtgacca

SEQ ID NO: 342 - DNA, 33 bp, Homo Sapiens
ttaggggtagaagaaargatggattgaggttgt

SEQ ID NO: 343 - DNA, 33 bp, Homo Sapiens
tatgagacacaattccrgaagaggaaaacagct

SEQ ID NO: 344 - DNA, 33 bp, Homo Sapiens
gatgctatagatctacrcactaaatgttgaata

SEQ ID NO: 345 - DNA, 33 bp, Homo Sapiens
aacccctatcagagccygagccgagtaagacag

SEQ ID NO: 346 - DNA, 33 bp, Homo Sapiens
ggcttccagttccaccmatgtttctgcaaagga

SEQ ID NO: 347 - DNA, 33 bp, Homo Sapiens
ccccattcacgggccaygttccaatcggcttac

SEQ ID NO: 348 - DNA, 33 bp, Homo Sapiens
tggaaatgaatccctcyctaatgtccgtaggaa

SEQ ID NO: 349 - DNA, 33 bp, Homo Sapiens
ttcctttacttttaacsgagtggagtttctgta

SEQ ID NO: 350 - DNA, 33 bp, Homo Sapiens
ggacatctgcttttagmccttcttccaagcagg

SEQ ID NO: 351 - DNA, 33 bp, Homo Sapiens
ttatgctatttgagagraaattcaaaccatgag

SEQ ID NO: 352 - DNA, 33 bp, Homo Sapiens
atatagggttatttccrgatgttgctatggcat

SEQ ID NO: 353 - DNA, 33 bp, Homo Sapiens
tttatagctgaaatggmaacaacttgttcttcc

SEQ ID NO: 354 - DNA, 33 bp, Homo Sapiens
aatctcttcactcttcraccgtatttccaagag

SEQ ID NO: 355 - DNA, 33 bp, Homo Sapiens
ggtaactactggtacamcttttgtgatgagcat

SEQ ID NO: 356 - DNA, 33 bp, Homo Sapiens
tgtaggtgagcaatccrggactacggaaatgca

SEQ ID NO: 357 - DNA, 33 bp, Homo Sapiens
ataattgcccaattcarcttgaaggcctgatac

SEQ ID NO: 358 - DNA, 33 bp, Homo Sapiens
aaatgctggatgactasgcagcttaattataga

SEQ ID NO: 359 - DNA, 33 bp, Homo Sapiens
ataactgccaagcaacragactggttctgggta

SEQ ID NO: 360 - DNA, 33 bp, Homo Sapiens
tcttgatgagctcaagsctaagtgctctctccc

SEQ ID NO: 361 - DNA, 33 bp, Homo Sapiens
aactcggggcaaaagcygatagtggatatagct

SEQ ID NO: 362 - DNA, 33 bp, Homo Sapiens
aagcagtcagaggccakgaaacagggcaaaagc

SEQ ID NO: 363 - DNA, 33 bp, Homo Sapiens
aagagtaaacattactsaaggatttttacctcc

SEQ ID NO: 364 - DNA, 33 bp, Homo Sapiens
acagataaagagacackgacgtaggaagctatg

SEQ ID NO: 365 - DNA, 33 bp, Homo Sapiens
aattgtccttaaactgsaaaatgagaaaataat

SEQ ID NO: 366 - DNA, 33 bp, Homo Sapiens
taatgataaattttaasatcttgaaagcagcca

SEQ ID NO: 367 - DNA, 33 bp, Homo Sapiens
agtagagacaaatgcckctaggggatgattatg

SEQ ID NO: 368 - DNA, 33 bp, Homo Sapiens
acaaagaccactggaamagaagctttgtcacca

SEQ ID NO: 369 - DNA, 33 bp, Homo Sapiens
atgcaggtggtcctagraatacactttggcaaa

SEQ ID NO: 370 - DNA, 33 bp, Homo Sapiens
aatattgaaatttcctsacatgacaacttacat

SEQ ID NO: 371 - DNA, 33 bp, Homo Sapiens
atcactctgtttgtaayatggagagccaattga

SEQ ID NO: 372 - DNA, 33 bp, Homo Sapiens
atacctgttaccttgaygattgggaccattttt

SEQ ID NO: 373 - DNA, 33 bp, Homo Sapiens
aagaatctggtaagaamtatagactccttctca

SEQ ID NO: 374 - DNA, 33 bp, Homo Sapiens
tcaatcaggagaaccayatctttgagaaggcag

SEQ ID NO: 375 - DNA, 33 bp, Homo Sapiens
aataatattagcccaarcagtggttctactcac

SEQ ID NO: 376 - DNA, 33 bp, Homo Sapiens
aggtcaccctacaaaasttatcttcaatgactc

SEQ ID NO: 377 - DNA, 33 bp, Homo Sapiens
ttgtctggacctaaaasaggacgagtgcttgga

SEQ ID NO: 378 - DNA, 33 bp, Homo Sapiens
agaaacctctgtgttcyatatgccaatcagtca

SEQ ID NO: 379 - DNA, 33 bp, Homo Sapiens
aattttacttcactcayatcactggcgtcaact

SEQ ID NO: 380 - DNA, 33 bp, Homo Sapiens
agagaaaggcggagaarcatctgtcaaatttaa

SEQ ID NO: 381 - DNA, 33 bp, Homo Sapiens
ggctactttatcaggaratacttttatttactt

SEQ ID NO: 382 - DNA, 33 bp, Homo Sapiens
gggtatttgcaaagcastgctgaacattaagga

SEQ ID NO: 383 - DNA, 33 bp, Homo Sapiens
gttatcatttaactgcrcttatgtgttagcaga

SEQ ID NO: 384 - DNA, 33 bp, Homo Sapiens
actaattgcattcacarccaagcaggatgattt

SEQ ID NO: 385 - DNA, 33 bp, Homo Sapiens
ttttttctcttgcatcyaggaaagacagatagc

SEQ ID NO: 386 - DNA, 33 bp, Homo Sapiens
agatagagaaagcaacrgtggcttaaaatgcaa

SEQ ID NO: 387 - DNA, 33 bp, Homo Sapiens
cttatttgtgagagaasagaaacctccagggtg

SEQ ID NO: 388 - DNA, 33 bp, Homo Sapiens
tcctggaggctgcctayagttccttaccacata

SEQ ID NO: 389 - DNA, 33 bp, Homo Sapiens
gatggattactcttcawgaccacagaagacttt

SEQ ID NO: 390 - DNA, 33 bp, Homo Sapiens
ttgggttgcaagtaacygagtagcttccattaa

SEQ ID NO: 391 - DNA, 33 bp, Homo Sapiens
gcctgtaattccagcawctcggcagtctgagga

SEQ ID NO: 392 - DNA, 33 bp, Homo Sapiens
ccaggtgttaagtgtcraaggaaatgtgccaag

SEQ ID NO: 393 - DNA, 33 bp, Homo Sapiens
ggtcacggatttcagcyctgatctaatcttgcc

SEQ ID NO: 394 - DNA, 33 bp, Homo Sapiens
tgacaggagcagttgaygtgacactcaaagtgg

SEQ ID NO: 395 - DNA, 33 bp, Homo Sapiens
aaagtggtaccatttargctgtagtgcaaagaa

SEQ ID NO: 396 - DNA, 33 bp, Homo Sapiens
aacagaaattcaacaaraggtccctgaagagct

SEQ ID NO: 397 - DNA, 33 bp, Homo Sapiens
cactccagcctggtcayacttaaacagacaaac

SEQ ID NO: 398 - DNA, 33 bp, Homo Sapiens
acaagcctgcaatacasagcacaaggataattg

SEQ ID NO: 399 - DNA, 33 bp, Homo Sapiens
ggaaccatgaaagatayaattgataacatggac

SEQ ID NO: 400 - DNA, 33 bp, Homo Sapiens
taaagttctttcaggcmaggaccatttctcatt

SEQ ID NO: 401 - DNA, 33 bp, Homo Sapiens
cttttctaagatctacrgtaagcatgttttaaa

SEQ ID NO: 402 - DNA, 33 bp, Homo Sapiens
accacatcactcagaargtacgtcccacttctt

SEQ ID NO: 403 - DNA, 33 bp, Homo Sapiens
tttattccaagcaaaasgggaacaaggttgcat

SEQ ID NO: 404 - DNA, 33 bp, Homo Sapiens
cccacactggtgtagcyccaagcgcttctgcca

SEQ ID NO: 405 - DNA, 33 bp, Homo Sapiens
caaacctctacactaayttagaagagaagaggc

SEQ ID NO: 406 - DNA, 33 bp, Homo Sapiens
ttgggaaatcaatgatraagaaactgcaatacc

SEQ ID NO: 407 - DNA, 33 bp, Homo Sapiens
ttcttctagacactcaraaaccatgatttagga

SEQ ID NO: 408 - DNA, 33 bp, Homo Sapiens
atttgttgttcctatawcatctgacttattccg

SEQ ID NO: 409 - DNA, 33 bp, Homo Sapiens
ttctggtttccagttcyacttataagtcatcac

SEQ ID NO: 410 - DNA, 33 bp, Homo Sapiens
gttccttgaaccacaargaataagcacagatct

SEQ ID NO: 411 - DNA, 33 bp, Homo Sapiens
tttaatgctttttataygtcacttgaaattttc

SEQ ID NO: 412 - DNA, 33 bp, Homo Sapiens
cataaatgaggagataygttagaatcctgggaa

SEQ ID NO: 413 - DNA, 33 bp, Homo Sapiens
gcaccctgatccaaccygggttctaaataggtc

SEQ ID NO: 414 - DNA, 33 bp, Homo Sapiens
agctgaggaatcttgtracaaaatagagaggac

SEQ ID NO: 415 - DNA, 33 bp, Homo Sapiens
tttagtactgtctataygctctttccagcccct

SEQ ID NO: 416 - DNA, 33 bp, Homo Sapiens
aatggcctctttcccaygcgtactgcccggtgg

SEQ ID NO: 417 - DNA, 33 bp, Homo Sapiens
gagaagacaaatatagmagtgccttgagttttt

SEQ ID NO: 418 - DNA, 33 bp, Homo Sapiens
tattccaaaccatgacmaatgcttctgataggg

SEQ ID NO: 419 - DNA, 33 bp, Homo Sapiens
cccaggctccttgctasaaaaattgccaggtca

SEQ ID NO: 420 - DNA, 33 bp, Homo Sapiens
ctttcagaatcctagcygcaggaggcctcacaa

SEQ ID NO: 421 - DNA, 33 bp, Homo Sapiens
acagcctttcttctcayaggacagacagccttc

SEQ ID NO: 422 - DNA, 33 bp, Homo Sapiens
tgccagctttacaagcsacagaatcaccaaaac

SEQ ID NO: 423 - DNA, 33 bp, Homo Sapiens
catgccatttcaccagyatgtagggatttttaa

SEQ ID NO: 424 - DNA, 33 bp, Homo Sapiens
ttcagtcgagtaacaartattcttgctggggtt

SEQ ID NO: 425 - DNA, 33 bp, Homo Sapiens
tagatcggttttgagasagagtaaactgttggg

SEQ ID NO: 426 - DNA, 33 bp, Homo Sapiens
aacactgggtaaatgcrgcatcagtgactaaga

SEQ ID NO: 427 - DNA, 33 bp, Homo Sapiens
aagatctggcctatggsaaaccttggcaggtgc

SEQ ID NO: 428 - DNA, 33 bp, Homo Sapiens
tcctcttccctggaccrctgcatacttttgtcc

SEQ ID NO: 429 - DNA, 33 bp, Homo Sapiens
ttctggcaggtctgaawtgagagaagaaaatgg

SEQ ID NO: 430 - DNA, 33 bp, Homo Sapiens
gccatgcttgctattayagaaaatgctgtgatt

SEQ ID NO: 431 - DNA, 33 bp, Homo Sapiens
attgcgacagtgccacrcctggacccagaggca

SEQ ID NO: 432 - DNA, 33 bp, Homo Sapiens
cgtaagagttccacaaygattcccactgctgaa

SEQ ID NO: 433 - DNA, 33 bp, Homo Sapiens
aaaaactcactcatgcrcaatgtaagccccaaa

SEQ ID NO: 434 - DNA, 33 bp, Homo Sapiens
aagaattacttgtcaastgcctactctgcaaca

SEQ ID NO: 435 - DNA, 33 bp, Homo Sapiens
atgcttatgtgtttaaygctccctcctcctcct

SEQ ID NO: 436 - DNA, 33 bp, Homo Sapiens
gtattagtatgttaggratgctgtaacaaaatc

SEQ ID NO: 437 - DNA, 33 bp, Homo Sapiens
gccagttgttaagtacmgccatgattaaaaggt

SEQ ID NO: 438 - DNA, 33 bp, Homo Sapiens
aatgaaaacattcagaygtcagaacatgaaagc

SEQ ID NO: 439 - DNA, 33 bp, Homo Sapiens
accagttaaactccaayatttggcctctcagtg

SEQ ID NO: 440 - DNA, 33 bp, Homo Sapiens
cttaaatgctataaaakggaaacagtgcataga

SEQ ID NO: 441 - DNA, 33 bp, Homo Sapiens
gaaattgaaactctgaraaattgtaggtaggaa

SEQ ID NO: 442 - DNA, 33 bp, Homo Sapiens
tggtttgtggttacaaygcaaaccttgattttt

SEQ ID NO: 443 - DNA, 33 bp, Homo Sapiens
ttctaaactgcttctakgtattattaaaccaaa

SEQ ID NO: 444 - DNA, 33 bp, Homo Sapiens
tatctatgaagatttamgtttgactcaagttta

SEQ ID NO: 445 - DNA, 33 bp, Homo Sapiens
atccactttgtaggcaycttttccagtgtttgc

SEQ ID NO: 446 - DNA, 33 bp, Homo Sapiens
agatcctcgacgcagcrcgcccattaggggagt

SEQ ID NO: 447 - DNA, 33 bp, Homo Sapiens
agacagacagtgagtawgaaaaagctgttgtct

SEQ ID NO: 448 - DNA, 33 bp, Homo Sapiens
gactacagaaatgcttyaaaacaagtattatga

SEQ ID NO: 449 - DNA, 33 bp, Homo Sapiens
aaaggagtacaatgaaragagttaagccaagtg

SEQ ID NO: 450 - DNA, 33 bp, Homo Sapiens
gccacaaaaccattgcsaattataactaattaa

SEQ ID NO: 451 - DNA, 33 bp, Homo Sapiens
gagattaatatgttcargtgtgaaattgtcagc

SEQ ID NO: 452 - DNA, 33 bp, Homo Sapiens
tggcaggggtgtcacaygagtatgaattaggca

SEQ ID NO: 453 - DNA, 33 bp, Homo Sapiens
accttgcttttctcatyatacttatagcttcca

SEQ ID NO: 454 - DNA, 33 bp, Homo Sapiens
atcagcatgttttggayacaatgtctgtgctag

SEQ ID NO: 455 - DNA, 33 bp, Homo Sapiens
ttgtgacacaaacagargccaatacagctgcat

SEQ ID NO: 456 - DNA, 33 bp, Homo Sapiens
aatactgaaggaaatawctaaaagtggaaagtg

SEQ ID NO: 457 - DNA, 33 bp, Homo Sapiens
aatctgacctggagcasagagctacttagctga

SEQ ID NO: 458 - DNA, 33 bp, Homo Sapiens
aaaggttgtgaaaatcraagtacttagatcaat

SEQ ID NO: 459 - DNA, 33 bp, Homo Sapiens
gcctaaggaggttgtcycagtttttgtcaggta

SEQ ID NO: 460 - DNA, 33 bp, Homo Sapiens
gagattcaaagacaagragttaatttccaaggt

SEQ ID NO: 461 - DNA, 33 bp, Homo Sapiens
agcctgagagtttgccwaagttaggttttatta

SEQ ID NO: 462 - DNA, 33 bp, Homo Sapiens
aaaaccaaggaaaatartagggatagactgatg

SEQ ID NO: 463 - DNA, 33 bp, Homo Sapiens
ccactgctactaaaaaytaacatccaggaggta

SEQ ID NO: 464 - DNA, 33 bp, Homo Sapiens
tttcttgttaaatgacmagtggcttacttaatt

SEQ ID NO: 465 - DNA, 33 bp, Homo Sapiens
tctggaataatgagggyattagcaacagaaata

SEQ ID NO: 466 - DNA, 33 bp, Homo Sapiens
gagtaatatttcattgwaagatgttccacgatt

SEQ ID NO: 467 - DNA, 33 bp, Homo Sapiens
aatgtagcttagtcaawtatagatatcacaaaa

SEQ ID NO: 468 - DNA, 33 bp, Homo Sapiens
gaattggagacaatttwaaatcattttgaacat

SEQ ID NO: 469 - DNA, 33 bp, Homo Sapiens
ggacatggattcaagakgtgtgaatttcctctt

SEQ ID NO: 470 - DNA, 33 bp, Homo Sapiens
tatgttgcttccaaackccaattttcaacttta

SEQ ID NO: 471 - DNA, 33 bp, Homo Sapiens
atttgtggtggaaaccracaagctgatgctata

SEQ ID NO: 472 - DNA, 33 bp, Homo Sapiens
ggaaagatgtaatcccwgaatagagggcagttg

SEQ ID NO: 473 - DNA, 33 bp, Homo Sapiens
cactcatcagccaacaygagtggcactctctaa

SEQ ID NO: 474 - DNA, 33 bp, Homo Sapiens
ctttgcattttctgtaygcctcatatacatata

SEQ ID NO: 475 - DNA, 33 bp, Homo Sapiens
cagaatatatggtcaayctctggaacattcgca

SEQ ID NO: 476 - DNA, 33 bp, Homo Sapiens
ctttatgctccaaagaygtgctgagacaaatgt

SEQ ID NO: 477 - DNA, 33 bp, Homo Sapiens
ttgcagaataattaccrgaaccttgctatctac

SEQ ID NO: 478 - DNA, 33 bp, Homo Sapiens
ggggctgctacttgcaytgacatttatcatttc

SEQ ID NO: 479 - DNA, 33 bp, Homo Sapiens
ggtgtaaaattaatccratatgtttcttaaggt

SEQ ID NO: 480 - DNA, 33 bp, Homo Sapiens
gagtgcaaatatttccracccgatactgccgtc

SEQ ID NO: 481 - DNA, 33 bp, Homo Sapiens
ctgaaatttgaaagctragcacaaacaggcctc

SEQ ID NO: 482 - DNA, 33 bp, Homo Sapiens
acaataagagagaaacmagtaaagctttggaaa

SEQ ID NO: 483 - DNA, 33 bp, Homo Sapiens
catcaggacagattaasatcaatctacaaacca

SEQ ID NO: 484 - DNA, 33 bp, Homo Sapiens
aaacatacttgctagayactggttcaactatga

SEQ ID NO: 485 - DNA, 33 bp, Homo Sapiens
ttaaggtatttttccartctattctagcctctg

SEQ ID NO: 486 - DNA, 33 bp, Homo Sapiens
ttattaaaaagcttgaraggccaggcaaagtgg

SEQ ID NO: 487 - DNA, 33 bp, Homo Sapiens
agccagatgatgatcaktctgattattttgtcc

SEQ ID NO: 488 - DNA, 33 bp, Homo Sapiens
tagagggaaagtgagaratcatttaatttaaag

SEQ ID NO: 489 - DNA, 33 bp, Homo Sapiens
atgaaaccttagaaaasgtgagaatggagcctt

SEQ ID NO: 490 - DNA, 33 bp, Homo Sapiens
tggcagtcatggcagcragcatatggatagcag

SEQ ID NO: 491 - DNA, 33 bp, Homo Sapiens
agaccagtttgctggawaactcagaagggagac

SEQ ID NO: 492 - DNA, 33 bp, Homo Sapiens
tttctgttaaagttcaycgtggcaatgtaaatt

SEQ ID NO: 493 - DNA, 33 bp, Homo Sapiens
acacattttgaaagccratgattttataagttt

SEQ ID NO: 494 - DNA, 33 bp, Homo Sapiens
gtagacaacaaccactyagtgagatattaaacc

SEQ ID NO: 495 - DNA, 33 bp, Homo Sapiens
cgtctttgcctgtcaamctcctctttagtcact

SEQ ID NO: 496 - DNA, 33 bp, Homo Sapiens
ccccaactgctcagcargtcattcaggccacct

SEQ ID NO: 497 - DNA, 33 bp, Homo Sapiens
cttgtgactgaatcacwatctttctgtggactt

SEQ ID NO: 498 - DNA, 33 bp, Homo Sapiens
tttctttactagtctgratagtggcctatttta

SEQ ID NO: 499 - DNA, 33 bp, Homo Sapiens
tccagtaaaaacttagractgttattagcatca

SEQ ID NO: 500 - DNA, 33 bp, Homo Sapiens
aagaatgtgccagaaaraagctgatactgagca

SEQ ID NO: 501 - DNA, 33 bp, Homo Sapiens
tctgctgctatgtccaygtgaccttggaaaagt

SEQ ID NO: 502 - DNA, 33 bp, Homo Sapiens
agaaagatatttcttgratgttaggagagaatg

SEQ ID NO: 503 - DNA, 33 bp, Homo Sapiens
gtaacagaccacctcamtaacagtggcttaagc

SEQ ID NO: 504 - DNA, 33 bp, Homo Sapiens
aaaaatcctaacaaatmaagaggccatgtatgc

SEQ ID NO: 505 - DNA, 33 bp, Homo Sapiens
tgtgtctgagaccaatyataaagtgacatacag

SEQ ID NO: 506 - DNA, 33 bp, Homo Sapiens
acacaaaccattcaatmaactgtgtgtggtaat

SEQ ID NO: 507 - DNA, 33 bp, Homo Sapiens
ctctgacactttatgcygagttgctaccatttc

SEQ ID NO: 508 - DNA, 33 bp, Homo Sapiens
tagacaagacttatgayatagagaagtaacgac

SEQ ID NO: 509 - DNA, 33 bp, Homo Sapiens
aagaggaagtataatcrcaatcttctgcttaaa

SEQ ID NO: 510 - DNA, 33 bp, Homo Sapiens
tcaaggatgccagataygtcccatgagatgggc

SEQ ID NO: 511 - DNA, 33 bp, Homo Sapiens
tgtgtagagaaccaagrctagagaacatatatt

SEQ ID NO: 512 - DNA, 33 bp, Homo Sapiens
aaagtcactcctaaaarcatatatcactcaatg

SEQ ID NO: 513 - DNA, 33 bp, Homo Sapiens
catcacacagctggtayatagatggtgccactg

SEQ ID NO: 514 - DNA, 33 bp, Homo Sapiens
aagatcagtgcagggastacgcttttaaagaac

SEQ ID NO: 515 - DNA, 33 bp, Homo Sapiens
tttatctcctaagtcayataagttgggcattat

SEQ ID NO: 516 - DNA, 33 bp, Homo Sapiens
agtaatggctcgttaartcaattaacatttgtc

SEQ ID NO: 517 - DNA, 33 bp, Homo Sapiens
ggaggtagaagcttacrattctgtgtggacaag

SEQ ID NO: 518 - DNA, 33 bp, Homo Sapiens
taagaataatttttggyatgggtaatatggcaa

SEQ ID NO: 519 - DNA, 33 bp, Homo Sapiens
ttgtgtgcatccatgtracatcgtaactcggtg

SEQ ID NO: 520 - DNA, 33 bp, Homo Sapiens
ccaatctttacctgacratgaattatcctcact

SEQ ID NO: 521 - DNA, 33 bp, Homo Sapiens
gttttctttgaggaagwataagctcacctgcat

SEQ ID NO: 522 - DNA, 33 bp, Homo Sapiens
cgtttgttttattgaargtgtgcattttcttct

SEQ ID NO: 523 - DNA, 33 bp, Homo Sapiens
atctcagctcctgcacsatctcagtcacgacct

SEQ ID NO: 524 - DNA, 33 bp, Homo Sapiens
gctctggagtgaaaccmccctgcgcttttatcc

SEQ ID NO: 525 - DNA, 33 bp, Homo Sapiens
ggcccatggatgtgaaycagcttgtctgatctg

SEQ ID NO: 526 - DNA, 33 bp, Homo Sapiens
tgattaacagctaaaayattcttacggccttta

SEQ ID NO: 527 - DNA, 33 bp, Homo Sapiens
gccatcctatctgccaraataaatcaagctttc

SEQ ID NO: 528 - DNA, 33 bp, Homo Sapiens
agtcctgcgagaataasaagtggacaagtaaaa

SEQ ID NO: 529 - DNA, 33 bp, Homo Sapiens
atgacttcgaatataaygacaaatcaatacaaa

SEQ ID NO: 530 - DNA, 33 bp, Homo Sapiens
atagtaaaatgatccaytcaagaaaagaagcac

SEQ ID NO: 531 - DNA, 33 bp, Homo Sapiens
ctacaaggaaatcaaayagctcaagaagaaaaa

SEQ ID NO: 532 - DNA, 33 bp, Homo Sapiens
gttatgatcttgcacayaaataaaagtgtcaca

SEQ ID NO: 533 - DNA, 33 bp, Homo Sapiens
cactcttgccttcctarcactcacgtaccattt

SEQ ID NO: 534 - DNA, 33 bp, Homo Sapiens
tcaaatcaacaatctcrctacacactcttatca

SEQ ID NO: 535 - DNA, 33 bp, Homo Sapiens
taggatgcttttgcagwaattaacttgagtgga

SEQ ID NO: 536 - DNA, 33 bp, Homo Sapiens
agtcagtataatcagaycaagaacaggtgagaa

SEQ ID NO: 537 - DNA, 33 bp, Homo Sapiens
ttctccccaccagaacmcaaagaatgggaagct

SEQ ID NO: 538 - DNA, 33 bp, Homo Sapiens
cagatggattctatctmactcagatgttatatc

SEQ ID NO: 539 - DNA, 33 bp, Homo Sapiens
atcctcttaacagtcckcaaatataggcattac

SEQ ID NO: 540 - DNA, 33 bp, Homo Sapiens
gacaatccggtgattgmaggtttgctgtacagt

SEQ ID NO: 541 - DNA, 33 bp, Homo Sapiens
gtataattatgggtaamtatttgataacatgac

SEQ ID NO: 542 - DNA, 33 bp, Homo Sapiens
aaaatatatagagttayaggcaattgattaagg

SEQ ID NO: 543 - DNA, 33 bp, Homo Sapiens
cttgggaaattctggayaccattgcagaactca

SEQ ID NO: 544 - DNA, 33 bp, Homo Sapiens
gaataggtgaccattayagtttatctctaacta

SEQ ID NO: 545 - DNA, 33 bp, Homo Sapiens
ctttccttcttgcaggyattcctaatttctgta

SEQ ID NO: 546 - DNA, 33 bp, Homo Sapiens
aaaatcatctgagcagrcaaatataactctttt

SEQ ID NO: 547 - DNA, 33 bp, Homo Sapiens
cagagtccaagctaaaytgatcggtgcagggca

SEQ ID NO: 548 - DNA, 33 bp, Homo Sapiens
acctacgctgctgtgartacccagacgaagtct

SEQ ID NO: 549 - DNA, 33 bp, Homo Sapiens
gcactgtgtatgatcgrccaagggggacttaga

SEQ ID NO: 550 - DNA, 33 bp, Homo Sapiens
gccctggacttcacaamaaaaacaaagcatata

SEQ ID NO: 551 - DNA, 33 bp, Homo Sapiens
tagagaacttggtgaaygtaaaaatgtattttc

SEQ ID NO: 552 - DNA, 33 bp, Homo Sapiens
ttctagccttttagtayggcaaatcatatttta

SEQ ID NO: 553 - DNA, 33 bp, Homo Sapiens
ccaaaatacacttctawtacaggagttatcgag

SEQ ID NO: 554 - DNA, 33 bp, Homo Sapiens
ggaagttagtgaacaaygatttaacatgaaatt

SEQ ID NO: 555 - DNA, 33 bp, Homo Sapiens
cttctctttgtgtccartaatctgaaatcttat

SEQ ID NO: 556 - DNA, 33 bp, Homo Sapiens
acagctgtgaacaaaamgacaataaccagtgtt

SEQ ID NO: 557 - DNA, 33 bp, Homo Sapiens
caataagatacatggaraaacactgatttcatc

SEQ ID NO: 558 - DNA, 33 bp, Homo Sapiens
acatggaacaaatacaraatggactatcgaatt

SEQ ID NO: 559 - DNA, 33 bp, Homo Sapiens
ggtggttcttggttacratagcccaaaaaaact

SEQ ID NO: 560 - DNA, 33 bp, Homo Sapiens
tttttgtttttaagacyctgtattcaaagtaat

SEQ ID NO: 561 - DNA, 33 bp, Homo Sapiens
ttttgccagactattgyatatttataatatgaa

SEQ ID NO: 562 - DNA, 33 bp, Homo Sapiens
tctttccctataactakgttgagaatgtaaaga

SEQ ID NO: 563 - DNA, 33 bp, Homo Sapiens
tctagcacagtagtttwaaacgttttctcatca

SEQ ID NO: 564 - DNA, 33 bp, Homo Sapiens
tagcccagggaaagtcycagtttactcctgtca

SEQ ID NO: 565 - DNA, 33 bp, Homo Sapiens
aaatatttagcatgtawgtaaaaaagttaatga

SEQ ID NO: 566 - DNA, 33 bp, Homo Sapiens
caatggacttgctcaakgtttaaatgctctaca

SEQ ID NO: 567 - DNA, 33 bp, Homo Sapiens
gacagaagtaaaaccasacttgcattaagatag

SEQ ID NO: 568 - DNA, 33 bp, Homo Sapiens
catgaactctttggccyatcacattactgacca

SEQ ID NO: 569 - DNA, 33 bp, Homo Sapiens
gaaggaagctcgcctcrgaatctatggcacagc

SEQ ID NO: 570 - DNA, 33 bp, Homo Sapiens
ttactcccatatccaayctatacatgatccaca

SEQ ID NO: 571 - DNA, 33 bp, Homo Sapiens
atgtaggaattaaaaamtgtgagctcacagata

SEQ ID NO: 572 - DNA, 33 bp, Homo Sapiens
gtgtggtgtcagctgarcccgaaaacctaaggt

SEQ ID NO: 573 - DNA, 33 bp, Homo Sapiens
caataatcaaatgaagkattaagacgtttgaaa

SEQ ID NO: 574 - DNA, 33 bp, Homo Sapiens
gcacttcaccatgtgcycctaatctaatgtctc

SEQ ID NO: 575 - DNA, 33 bp, Homo Sapiens
ataaggaaaacaattaygtctctacacaaaaaa

SEQ ID NO: 576 - DNA, 33 bp, Homo Sapiens
aagtatgtaattgggaygatgggggtcagaaag

SEQ ID NO: 577 - DNA, 33 bp, Homo Sapiens
gttgaagtgcaaatacrccccaccccagacctg

SEQ ID NO: 578 - DNA, 33 bp, Homo Sapiens
tagttgataccaatatwacaggatgtttttatc

SEQ ID NO: 579 - DNA, 33 bp, Homo Sapiens
caggagtacatttaggscagcggaatgtgttat

SEQ ID NO: 580 - DNA, 33 bp, Homo Sapiens
aagacattagaacagakatggccagggttttcc

SEQ ID NO: 581 - DNA, 33 bp, Homo Sapiens
caaaagctaggaagcasagcaatacttttaagt

SEQ ID NO: 582 - DNA, 33 bp, Homo Sapiens
tttgactcaggaaatawgaaatttagacaggac

SEQ ID NO: 583 - DNA, 33 bp, Homo Sapiens
agattaggatgctgtcrcagtattttttgggga

SEQ ID NO: 584 - DNA, 33 bp, Homo Sapiens
ttccctgatgacaacaytgaagcacacacatgc

SEQ ID NO: 585 - DNA, 33 bp, Homo Sapiens
gagttgaagctgcaaarttgttttggaatatcg

SEQ ID NO: 586 - DNA, 33 bp, Homo Sapiens
tttccttgaaacagagwaacagaaaactgccag

SEQ ID NO: 587 - DNA, 33 bp, Homo Sapiens
ggattatacttggttaygaaatttgggtgggca

SEQ ID NO: 588 - DNA, 33 bp, Homo Sapiens
actcatgatgtgcgacraagagggagtggcgtg

SEQ ID NO: 589 - DNA, 33 bp, Homo Sapiens
tgaccagtcatgcgaaygtttctgtagcaaggt

SEQ ID NO: 590 - DNA, 33 bp, Homo Sapiens
cctttccccgattccgyagtcattgacatttta

SEQ ID NO: 591 - DNA, 33 bp, Homo Sapiens
cagcgtgaaggagctgyactcataaaggtcatg

SEQ ID NO: 592 - DNA, 33 bp, Homo Sapiens
tcttatgtcctttgacrcgactcccccagctgg

SEQ ID NO: 593 - DNA, 33 bp, Homo Sapiens
cctcaccaaagttaccrcacaatgggtccacag

SEQ ID NO: 594 - DNA, 33 bp, Homo Sapiens
catatcaccaggtaaayactgagctaacttggt

SEQ ID NO: 595 - DNA, 33 bp, Homo Sapiens
tatgttgctatctatamcattgagggcatagtt

SEQ ID NO: 596 - DNA, 33 bp, Homo Sapiens
gatctagcaggtgtagkaagatgtcacctaagt

SEQ ID NO: 597 - DNA, 33 bp, Homo Sapiens
atcaactaggagtgagraagattcttagacact

SEQ ID NO: 598 - DNA, 33 bp, Homo Sapiens
tagcctgtccaccatayctcctcatatgaagca

SEQ ID NO: 599 - DNA, 33 bp, Homo Sapiens
cttctgctaaatagagwagtgttggtatttgtt

SEQ ID NO: 600 - DNA, 33 bp, Homo Sapiens
atgtaccagcagcatcrcttcataaaacaaagg

SEQ ID NO: 601 - DNA, 33 bp, Homo Sapiens
tctaattcccatccggyccacagagccccagag

SEQ ID NO: 602 - DNA, 33 bp, Homo Sapiens
aaccttacaagccaagkagccattgtggtagtt

SEQ ID NO: 603 - DNA, 33 bp, Homo Sapiens
aacatattgtacatcaygtgagtcacacatttc

SEQ ID NO: 604 - DNA, 33 bp, Homo Sapiens
ccttttccaaaccagarccaaaagttgcagata

SEQ ID NO: 605 - DNA, 33 bp, Homo Sapiens
cagtattcagaggaaaragtgttcctgagttgc

SEQ ID NO: 606 - DNA, 33 bp, Homo Sapiens
acgatgagatttacaargagttttcccagaggc

SEQ ID NO: 607 - DNA, 33 bp, Homo Sapiens
ctcttccttctgcctaraatattaacatctttg

SEQ ID NO: 608 - DNA, 33 bp, Homo Sapiens
tcctacatagctaacaytacaagagcatgccac

SEQ ID NO: 609 - DNA, 33 bp, Homo Sapiens
taggaaaactgcagccsagggaggttaaataac

SEQ ID NO: 610 - DNA, 33 bp, Homo Sapiens
acatcacttgccactaycattgctgtttgactt

SEQ ID NO: 611 - DNA, 33 bp, Homo Sapiens
gtgtagtatttatctckgagaactaaacctatg

SEQ ID NO: 612 - DNA, 33 bp, Homo Sapiens
agggattttccttaccwgggttgattatttaca

SEQ ID NO: 613 - DNA, 33 bp, Homo Sapiens
gagatgaagtatttcaygttggtcagttgtgcc

SEQ ID NO: 614 - DNA, 33 bp, Homo Sapiens
agtaattccagataagmaagtgcctcagttatt

SEQ ID NO: 615 - DNA, 33 bp, Homo Sapiens
ttgaggatgttagttaygtactgtcacatcaca

SEQ ID NO: 616 - DNA, 33 bp, Homo Sapiens
cataattagaagacgamtacaactgcattattt

SEQ ID NO: 617 - DNA, 33 bp, Homo Sapiens
cagggaattgaaactcragagcttagcttcaaa

SEQ ID NO: 618 - DNA, 33 bp, Homo Sapiens
cttgggtggtgcttacmggagctccatgaatag

SEQ ID NO: 619 - DNA, 33 bp, Homo Sapiens
gtatattgaagggttcyctattagtcagcctgc

SEQ ID NO: 620 - DNA, 33 bp, Homo Sapiens
cagttcacccagagcckgagattcctccacatc

SEQ ID NO: 621 - DNA, 33 bp, Homo Sapiens
atgtggatttcatggayacttttcaatgcgtat

SEQ ID NO: 622 - DNA, 33 bp, Homo Sapiens
cctttctctcacttcaygcaggcgctccatcct

SEQ ID NO: 623 - DNA, 33 bp, Homo Sapiens
atttgcaccactgttaraaggcagaaccccagc

SEQ ID NO: 624 - DNA, 33 bp, Homo Sapiens
cccacctgaatgttacrccggacgggagtatgg

SEQ ID NO: 625 - DNA, 33 bp, Homo Sapiens
tatgttggctgatgtawcttaatgtgcacatta

SEQ ID NO: 626 - DNA, 33 bp, Homo Sapiens
tttgtaaaagcagaagrctaaagacagtagaag

SEQ ID NO: 627 - DNA, 33 bp, Homo Sapiens
tagaactcacatgagaracgatactcaacttta

SEQ ID NO: 628 - DNA, 33 bp, Homo Sapiens
tcatctatgtgtgataytagataattagagttc

SEQ ID NO: 629 - DNA, 33 bp, Homo Sapiens
aatttgtttgtagatawgaatacatctgttttc

SEQ ID NO: 630 - DNA, 33 bp, Homo Sapiens
catgtacatcaaggcarcccaaccttctcttgg

SEQ ID NO: 631 - DNA, 33 bp, Homo Sapiens
gtacgtggtaagagcamctcaaagatattcttt

SEQ ID NO: 632 - DNA, 33 bp, Homo Sapiens
gcactaagcaaatcatraaagtggcatcagata

SEQ ID NO: 633 - DNA, 33 bp, Homo Sapiens
tactgcttacctaaaawctagtccagggtctct

SEQ ID NO: 634 - DNA, 33 bp, Homo Sapiens
aaccaagatggctcacygcccagaagaccacat

SEQ ID NO: 635 - DNA, 33 bp, Homo Sapiens
acaggcctagctcaaartaaaggagcaaggcac

SEQ ID NO: 636 - DNA, 33 bp, Homo Sapiens
cctatcaatattgcaakcctatttttaagcctg

SEQ ID NO: 637 - DNA, 33 bp, Homo Sapiens
cacttctctaacttccyaactgcggccccagga

SEQ ID NO: 638 - DNA, 33 bp, Homo Sapiens
ctgtgctggatgcttcrcttgttctaaagcagg

SEQ ID NO: 639 - DNA, 33 bp, Homo Sapiens
caaagtggttcccaaastatgcatatatcaaaa

SEQ ID NO: 640 - DNA, 33 bp, Homo Sapiens
gcaatgtctctttctgraacatgtatctggctg

SEQ ID NO: 641 - DNA, 33 bp, Homo Sapiens
accactgtcctcagatractaggcatgtcatat

SEQ ID NO: 642 - DNA, 33 bp, Homo Sapiens
ccgtctagactggtgcrcaacctcctccctctc

SEQ ID NO: 643 - DNA, 33 bp, Homo Sapiens
taaacagcaaaaactarctgaatcaccagagga

SEQ ID NO: 644 - DNA, 33 bp, Homo Sapiens
tgttatatctattcagwatgttatgttgcaatt

SEQ ID NO: 645 - DNA, 33 bp, Homo Sapiens
cccgacgtacacatacyaaaggacccagaaatc

SEQ ID NO: 646 - DNA, 33 bp, Homo Sapiens
aaagaagagccgagaayggatgtgggggcaaac

SEQ ID NO: 647 - DNA, 33 bp, Homo Sapiens
taagagtgagtttccayacagaatatcagttaa

SEQ ID NO: 648 - DNA, 33 bp, Homo Sapiens
accattataaactcacyacttttttttgttatg

SEQ ID NO: 649 - DNA, 33 bp, Homo Sapiens
aagcttccttccaaaaygactgctccgtttact

SEQ ID NO: 650 - DNA, 33 bp, Homo Sapiens
ttgtttatgaatgaccragtttcagataaaatc

SEQ ID NO: 651 - DNA, 33 bp, Homo Sapiens
gaaggttacagaacacmaagcatatataacaca

SEQ ID NO: 652 - DNA, 33 bp, Homo Sapiens
caacttttatggatagyagcaatgtgtgacttt

SEQ ID NO: 653 - DNA, 33 bp, Homo Sapiens
tcagttattcttccacrcagtcatataacatag

SEQ ID NO: 654 - DNA, 33 bp, Homo Sapiens
cagtcacagcaagataygctacatgattccact

SEQ ID NO: 655 - DNA, 33 bp, Homo Sapiens
ctgaagaaccattacartcagatttacatttat

SEQ ID NO: 656 - DNA, 33 bp, Homo Sapiens
ttatgttatagaacaartgctaaagaagtacta